(19) United States
(12) Patent Application Publication
(10) Pub. No.: US 2020/0015499 A1
(43) Pub. Date: Jan. 16, 2020

(54) TRYPSIN-LIKE SERINE PROTEASES AND USES THEREOF

(71) Applicant: DUPONT NUTRITION BIOSCIENCES APS, Copenhagen K (DK)

(72) Inventors: Stephen Shipovskov, EGÅ (DK); Zhengzheng Zou, Shanghai (CN); Shukun Yu, Malmo (SE); Xiaogang Gu, Palo Alto, CA (US)

(21) Appl. No.: 16/491,388

(22) PCT Filed: Mar. 9, 2018
(86) PCT No.: PCT/US18/21741

§ 371 Date: Sep. 5, 2019

(30) Foreign Application Priority Data
Mar. 15, 2017 (CN) PCT/CN2017/076768

(51) Int. Cl. A23K 20/189; C12N 9/52

(52) U.S. Cl. CPC A23K 20/189; C12Y 304/21; C12N 9/52

(57) ABSTRACT

Novel trypsin-like serine proteases and uses thereof are described herein.

### CROSS-REFERENCE TO RELATED APPLICATION

The present application claims priority to PCT/CN2017/076768, filed on Mar. 15, 2017 which is hereby incorporated by reference in its entirety.

### INCORPORATION BY REFERENCE OF THE SEQUENCE LISTING

The sequence listing provided in the file named NB40985WOPCT_SequenceListing_ST25 with a size of 131 KB which was created on Mar. 8, 2017 and which is filed herewith, is incorporated by reference herein in its entirety.

### FIELD

The field relates to novel trypsin-like serine proteases and uses thereof.

### BACKGROUND

Proteases (also called peptidases or proteinases) are enzymes capable of cleaving peptide bonds. Proteases have evolved multiple times, and different classes of proteases can perform the same reaction by completely different catalytic mechanisms. Proteases can be found in animals, plants, fungi, bacteria, archaea and viruses.

Proteolysis can be achieved by enzymes currently classified into six broad groups: aspartyl proteases, cysteine proteases, serine proteases (such as, e.g., subtilisins or trypsin-like proteases), threonine proteases, glutamic proteases, and metalloproteases.

Serine proteases are a subgroup of carbonyl hydrolases comprising a diverse class of enzymes having a wide range of specificities and biological functions. Notwithstanding this functional diversity, the catalytic machinery of serine proteases has been approached by at least two genetically distinct families of enzymes: 1) the subtilisins; and 2) trypsin-like serine proteases (also known as chymotrypsin-related). These two families of serine proteases or serine endopeptidases have very similar catalytic mechanisms. The tertiary structure of these two enzyme families brings together a conserved catalytic triad of amino acids consisting of serine, histidine and aspartate.

Much research has been conducted on the serine proteases, in particular, subtilisins due largely to their useful in industrial applications. Additional work has been focused on adverse environmental conditions (e.g., exposure to oxidative agents, chelating agents, extremes of temperature and/or pH) which can adversely impact the functionality of these enzymes in a variety of applications.

Thus, there is a continuing need to find new serine proteases such as trypsin-like proteases of prokaryotic origins which can be used under adverse conditions and retain or have improved proteolytic activity and/or stability.

### SUMMARY

In a first embodiment, there is described an isolated polypeptide having serine protease activity, selected from the group consisting of:

a) a polypeptide having an amino acid sequence of at least 91% identity with the amino acid sequence of SEQ ID NO:22;

b) a polypeptide having an amino acid sequence of at least 94% identity with the amino acid sequence of SEQ ID NO:23;

c) a polypeptide having an amino acid sequence of at least 98% identity with the amino acid sequence of SEQ ID NO:24;

d) a polypeptide having an amino acid sequence of at least 80% identity with the amino acid sequence of SEQ ID NO:25.

In a second embodiment, there is described an isolated polypeptide having serine protease activity comprising a predicted precursor amino acid sequence selected from: SEQ ID NO:3; SEQ ID NO:6; SEQ ID NO:9; and SEQ ID NO:12.

In a third embodiment, there is described an isolated polypeptide having serine protease activity comprising a protease catalytic region, selected from:

a) an amino acid sequence with at least 96% identity with the amino acid sequence of SEQ ID NO:18;

b) an amino acid sequence with at least 98% identity with the amino acid sequence of SEQ ID NO:19;

c) an amino acid sequence of SEQ ID NO:20;

d) an amino acid sequence with at least 91% identity with the amino acid sequence of SEQ ID NO:21;

In a fourth embodiment, there is described a recombinant construct comprising a regulatory sequence functional in a production host operably linked to a nucleotide sequence encoding at least one polypeptide having serine protease activity selected from the group consisting of:

a) a polypeptide comprising an amino acid sequence with at least 91% identity with the amino acid sequence of SEQ ID NO:22;

b) a polypeptide comprising an amino acid sequence with at least 94% identity with the amino acid sequence of SEQ ID NO:23;

c) a polypeptide comprising an amino acid sequence with at least 98% identity with the amino acid sequence of SEQ ID NO:24;

d) a polypeptide comprising an amino acid sequence with at least 80% identity with the amino acid sequence of SEQ ID NO:25.

The production host can be a host is selected from the group consisting of fungi, bacteria, and algae.

In a fifth embodiment, there is described a method for producing at least one serine protease comprising:

(a) transforming a production host with the recombinant construct described herein; and

(b) culturing the production host of step (a) under conditions whereby the at least one polypeptide having serine protease activity is produced.

According to this method at least one polypeptide having serine protease is optionally recovered from the production host.

In another aspect, a serine protease-containing culture supernatant can be obtained using any of the methods described herein.

In a still another aspect, the recombinant microbial production host for expressing at least one polypeptide having serine protease activity, comprises a recombinant construct as described herein.

Furthermore, the production host is selected from the group consisting of bacteria, fungi and algae.

In a sixth embodiment, there is described animal feed comprising at least one polypeptide having serine protease activity described herein wherein the serine protease is present in an amount from 1-20 g/ton feed.

Furthermore, this animal feed can comprise (a) at least one direct fed microbial, or (at least one other enzyme, or (c) at least one direct fed microbial and at least one other enzyme.

In a seventh embodiment, there is described a feed, feedstuff, a feed additive composition or premix comprising at least one of the polypeptides having serine protease activity described herein. Furthermore, this feed, feedstuff, feed additive composition or premix described herein can comprise (a) at least one direct fed microbial, or (at least one other enzyme, or (c) at least one direct fed microbial and at least one other enzyme.

In a seventh embodiment, there is a feed additive composition as described herein wherein said composition further comprises at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabisulfite, methyl paraben and propyl paraben.

In an eighth embodiment, there is described a granulated feed additive composition for use in animal feed comprising at least one polypeptide having serine protease activity as described herein, wherein the granulated feed additive composition comprises particles produced by a process selected from the group consisting of high shear granulation, drum granulation, extrusion, spheronization, fluidized bed agglomeration, fluidized bed spray coating, spray drying, freeze drying, prilling, spray chilling, spinning disk atomization, coacervation, tableting, or any combination of the above processes.

In another embodiment, the particles of this granulated feed additive composition can have a mean diameter of greater than 50 microns and less than 2000 microns.

In another aspect, this feed additive composition can be in a liquid form and, furthermore, is in a liquid form suitable for spray-drying on a feed pellet.

### BRIEF DESCRIPTION OF THE DRAWINGS AND SEQUENCES

FIG. 1. Plasmid AprE-SspCPro29 for expression of AprE-SspCPro29 protease.

FIG. 2. Enzyme activity dose responses of serine proteases SspCPro29, SspCPro33 and Pro Act on AAPF-pNA substrate.

FIG. 3. pH profile of serine proteases SspCPro23, SspCPro29. SspCPro33 and SspCPro59.

FIG. 4. Temperature profile of serine proteases SspCPro23, SspCPro29. SspCPro33 and SspCPro59.

FIG. 5A. Hydrolysis of corn soy meal detected by OPA for serine proteases SspCPro29 and SspCPro33 at pH 6.

FIG. 5B. Hydrolysis of corn soy meal detected by BCA for serine proteases SspCPro29 and SspCPro33 at pH 6.

FIG. 6. Cleaning performance of SspCPro29 and SspCPro33 proteases in GSM-B ADW detergent at pH 10.3.

FIG. 7. Cleaning performance of SspCPro29, SspCPro33 and BPN′Y217L proteases in liquid laundry detergent at 16° C.

FIG. 8. Cleaning performance of SspCPro29, SspCPro33 and BPN′Y217L proteases in liquid laundry detergent at 32° C.

FIG. 9. Cleaning performance of SspCPro29, SspCPro33 and GG36 proteases in powder laundry detergent at 16° C.

FIG. 10. Cleaning performance of SspCPro29, SspCPro33 and GG36 proteases in powder laundry detergent at 32° C.

FIGS. 11A-11D. Multiple sequence alignment of full length sequence of various Streptomyces sp trypsin-like serine proteases.

FIGS. 12A-12B. Multiple sequence alignment of predicted catalytic core sequences of Streptomyces sp trypsin-like serine proteases.

The following sequences comply with 37 C.F.R. §§ 1.821-1.825 (“Requirements for Patent applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the European Patent Convention (EPC) and the Patent Cooperation Treaty (PCT) Rules 5.2 and 49.5 (a-bis), and Section 208 and Annex C of the Administrative Instructions. The symbols and format used for nucleotide and amino acid sequence data comply with the rules set forth in 37 C.F.R. § 1.822.

SEQ ID NO:1 sets forth the nucleotide sequence of the SspCPro29 gene isolated from Streptomyces sp. C009.

SEQ ID NO:2 sets forth the predicted signal sequence of the SspCPro29 precursor protein.

SEQ ID NO:3 sets forth the amino acid sequence of the SspCPro29 precursor protein.

SEQ ID NO:4 sets forth the nucleotide sequence of the SspCPro33 gene isolated from Streptomyces sp. C001.

SEQ ID NO:5 sets forth the predicted signal sequence of the SspCPro33 precursor protein.

SEQ ID NO:6 sets forth the amino acid sequence of the SspCPro33 precursor protein. SEQ ID NO:7 sets forth the nucleotide sequence of the SspCPro23 gene isolated from Streptomyces sp. C003.

SEQ ID NO:8 sets forth the predicted signal sequence of the SspCPro23 precursor protein.

SEQ ID NO:9 sets forth the amino acid sequence of the SspCPro23 precursor protein. SEQ ID NO:10 sets forth the nucleotide sequence of the SspCPro59 gene isolated from Streptomyces sp. C055.

SEQ ID NO:11 sets forth the predicted signal sequence of the SspCPro59 precursor protein.

SEQ ID NO:12 sets forth the amino acid sequence of the SspCPro59 precursor protein.

SEQ ID NO: 13 sets forth the nucleotide sequences of synthetic AprE-SspCPro23,

SEQ ID NO: 14 sets forth the nucleotide sequences of AprE-SspCPro29,

SEQ ID NO: 15 sets forth the nucleotide sequences of AprE-SspCPro33,

SEQ ID NO: 16 sets forth the nucleotide sequences of AprE-SspCPro59 gene.

SEQ ID NO: 17 sets forth the AprE signal sequence that was used to direct the recombinant proteins for secretion in B. subtilis.

SEQ ID NO: 18 sets forth the predicted catalytic domain for SspCPro29.

SEQ ID NO: 19 sets forth the predicted catalytic domain for SspCPro 23.

SEQ ID NO: 20 sets forth the predicted catalytic domain for SspCPro 33.

SEQ ID NO: 21 sets forth the predicted catalytic domain for SspCPro 59.

SEQ ID NO:22 sets forth the predicted full length amino acid sequence for SspCPro29.

SEQ ID NO:23 sets forth the predicted full length amino acid sequence for SspCPro33.

SEQ ID NO:24 sets forth the predicted full length amino acid sequence for SspCPro23.

SEQ ID NO:25 sets forth the predicted full length amino acid sequence for SspCPro59.

SEQ ID NO:26 sets forth the sequence of Streptomyces sp serine protease WP_064069271.

SEQ ID NO:27 sets forth the sequence of Streptomyces sp serine protease WP_043225562.

SEQ ID NO:28 sets forth the sequence of Streptomyces sp serine protease WP_024756173.

SEQ ID NO:29 sets forth the sequence of Streptomyces sp serine protease WP_030548298.

SEQ ID NO:30 sets forth the sequence of Streptomyces sp serine protease WP_005320871.

SEQ ID NO:31 sets forth the sequence of Streptomyces sp serine protease WP_055639793.

SEQ ID NO:32 sets forth the sequence of Streptomyces sp serine protease WO2015048332-44360.

SEQ ID NO:33 sets forth the sequence of Streptomyces sp serine protease WO2015048332-44127.

SEQ ID NO:34 sets forth the sequence of Streptomyces sp serine protease WP_030313004.

SEQ ID NO:35 sets forth the sequence of Streptomyces sp serine protease WP_030212164.

SEQ ID NO:36 sets forth the sequence of Streptomyces sp serine protease WP_030749137.

SEQ ID NO:37 sets forth the sequence of Streptomyces sp serine protease WP_031004112.

SEQ ID NO:38 sets forth the sequence of Streptomyces sp serine protease WP_026277977.

SEQ ID NO:39 sets forth the amino acid sequence of the catalytic domain of Streptgrisin C.

SEQ ID NO:40 sets forth the amino acid sequence of BPN′-Y217L protein.

SEQ ID NO:41 sets forth the amino acid sequence of GG36 protein.

SEQ ID NO:42 sets forth the amino acid sequence of residues 204-394 pf S_albulus WP 064069271.

SEQ ID NO:43 sets forth the amino acid sequence of residues 204-394 of S_sp_NRRL_F-5193_WP_043225562 protein.

SEQ ID NO:44 sets forth the amino acid sequence of residues 201-391 of S_exfoliatus_WP_024756173.

SEQ ID NO:45 sets forth the amino acid sequence of residues 207-397 of S_albus_WP_030548298.

SEQ ID NO:46 sets forth the amino acid sequence of residues 204-394 of S_pristinaespiralis_WP_005320871.

SEQ ID NO:47 sets forth the amino acid sequence of residues 138-328 of S_leeuwenhoekii_WP_029386953.

SEQ ID NO:48 sets forth the amino acid sequence of residues 207-397 of Streptomyces sp. CNT372_WP_026277977.

SEQ ID NO:49 sets forth the amino acid sequence residues 208-398 of Streptomyces cyaneogriseus_P_044383230.

SEQ ID NO:50 sets forth the amino acid sequence of residues 193-383 of Streptomyces niveus WP_069630550.

SEQ ID NO:51 sets forth the amino acid sequence of residues 201-391 of Streptomyces venezuelae WP_055639793.

SEQ ID NO:52 sets forth the amino acid sequence of residues 211-401 of Streptomyces sp. NRRL F-5755 WP_053699044

SEQ ID NO:53 sets forth the amino acid sequence of residues 205-395 of Streptomyces fradiae_WP_031135572.

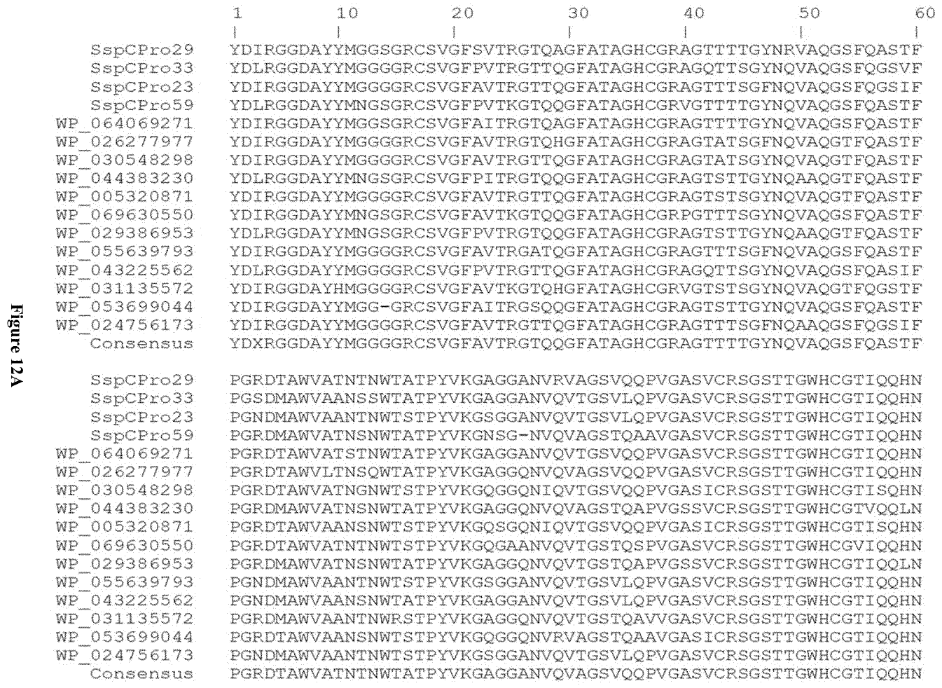

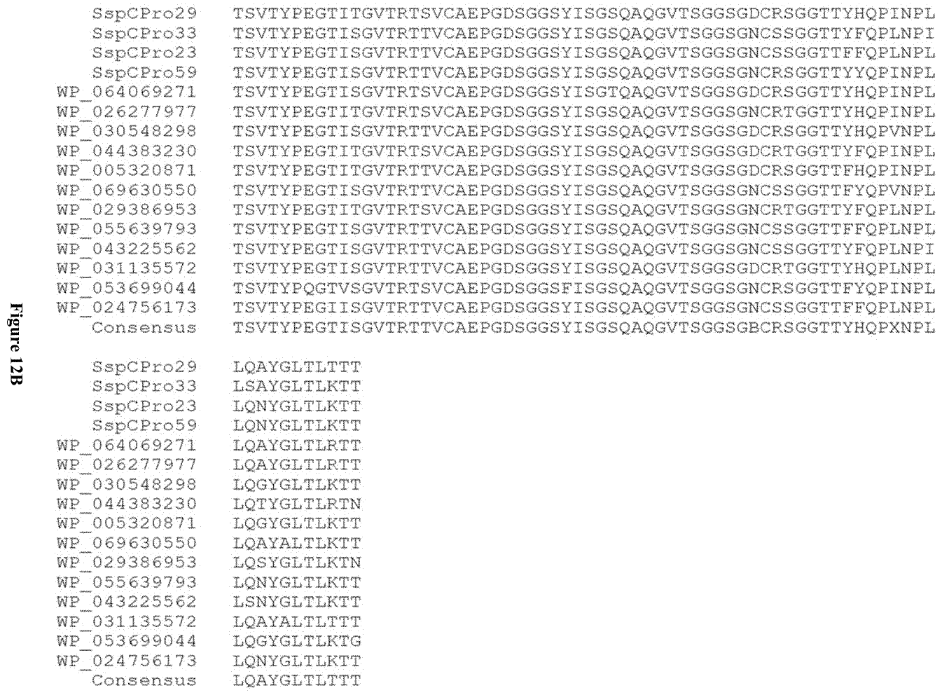

SEQ ID NO 54 sets forth the predicted catalytic domain consensus sequence from FIG. 12.

### DETAILED DESCRIPTION

All patents, patent applications, and publications cited are incorporated herein by reference in their entirety.

In this disclosure, a number of terms and abbreviations are used. The following definitions apply unless specifically stated otherwise.

The articles “a”, “an”, and “the” preceding an element or component are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

The term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein in connection with a numerical value, the term “about” refers to a range of +/−0.5 of the numerical value, unless the term is otherwise specifically defined in context. For instance, the phrase a “pH value of about 6” refers to pH values of from 5.5 to 6.5, unless the pH value is specifically defined otherwise.

It is intended that every maximum numerical limitation given throughout this Specification includes every lower numerical limitation, as if such lower numerical limitations were expressly written herein. Every minimum numerical limitation given throughout this Specification will include every higher numerical limitation, as if such higher numerical limitations were expressly written herein. Every numerical range given throughout this Specification will include every narrower numerical range that falls within such broader numerical range, as if such narrower numerical ranges were all expressly written herein.

The term “protease” means a protein or polypeptide domain derived from a microorganism, e.g., a fungus, bacterium, or from a plant or animal, and that has the ability to catalyze cleavage of peptide bonds at one or more of various positions of a protein backbone (e.g., E. C. 3.4). The terms “protease”, “peptidase” and “proteinase” can be used interchangeably. Proteases can be found in animals, plants, fungi, bacteria, archaea and viruses. Proteolysis can be achieved by enzymes currently classified into six broad groups based on their catalytic mechanisms: aspartyl proteases, cysteine proteases, trypsin-like serine proteases, threonine proteases, glutamic proteases, and metalloproteases.

The term “serine protease” refers to enzymes that cleave peptide bonds in proteins, in which serine serves as the nucleophilic amino acid at the active site of the enzyme. Serine proteases fall into two broad categories based on their structure: the chymotrypsin-like (trypsin-like) and the subtilisins. In the MEROPS protease classification system, proteases are distributed among 16 superfamilies and numerous families. The family S8 includes the subtilisins and the family 51 includes the chymotrypsin-like (trypsin-like) enzymes. The subfamily S1E includes the trypsin-like serine proteases from Streptomyces organisms, such as Streptogricins A, B and C. The terms “serine protease”, “trypsin-like serine protease” and “chymotrypsin-like protease” are used interchangeably herein.

The terms “animal” and “subject” are used interchangeably herein. An animal includes all non-ruminant (including humans) and ruminant animals. In a particular embodiment, the animal is a non-ruminant animal, such as a horse and a mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment the animal is a ruminant animal including, but not limited to, cattle, young calves, goats, sheep, giraffes, bison, moose, elk, yaks, water buffalo, deer, camels, alpacas, llamas, antelope, pronghorn and nilgai.

A “feed” and a “food,” respectively, means any natural or artificial diet, meal or the like or components of such meals intended or suitable for being eaten, taken in, digested, by a non-human animal and a human being, respectively.

As used herein, the term “food” is used in a broad sense and covers food and food products for humans as well as food for non-human animals (i.e. a feed).

The term “feed” is used with reference to products that are fed to animals in the rearing of livestock. The terms “feed” and “animal feed” are used interchangeably.

The term “direct-fed microbial” (“DFM”) as used herein is source of live (viable) naturally occurring microorganisms. A DFM can comprise one or more of such naturally occurring microorganisms such as bacterial strains. Categories of DFMs include Bacillus, Lactic Acid Bacteria and Yeasts. Thus, the term DFM encompasses one or more of the following: direct fed bacteria, direct fed yeast, direct fed yeast and combinations thereof.

Bacilli are unique, gram-positive rods that form spores. These spores are very stable and can withstand environmental conditions such as heat, moisture and a range of pH. These spores germinate into active vegetative cells when ingested by an animal and can be used in meal and pelleted diets. Lactic Acid Bacteria are gram-positive cocci that produce lactic acid which are antagonistic to pathogens. Since Lactic Acid Bacteria appear to be somewhat heat-sensitive, they are not used in pelleted diets. Types of Lactic Acid Bacteria include Bifidobacterium, Lactobacillus and Streptococcus.

The term “prebiotic” means a non-digestible food ingredient that beneficially affects the host by selectively stimulating the growth and/or the activity of one or a limited number of beneficial bacteria.

The term “probiotic culture” as used herein defines live microorganisms (including bacteria or yeasts for example) which, when for example ingested or locally applied in sufficient numbers, beneficially affects the host organism, i.e. by conferring one or more demonstrable health benefits on the host organism. Probiotics may improve the microbial balance in one or more mucosal surfaces. For example, the mucosal surface may be the intestine, the urinary tract, the respiratory tract or the skin. The term “probiotic” as used herein also encompasses live microorganisms that can stimulate the beneficial branches of the immune system and at the same time decrease the inflammatory reactions in a mucosal surface, for example the gut. Whilst there are no lower or upper limits for probiotic intake, it has been suggested that at least 10⁶-10¹², preferably at least 10⁶-10¹⁰, preferably 10⁸-10⁹, cfu as a daily dose will be effective to achieve the beneficial health effects in a subject.

The term “CFU” as used herein means “colony forming units” and is a measure of viable cells in which a colony represents an aggregate of cells derived from a single progenitor cell.

The term “isolated” means a substance in a form or environment that does not occur in nature. Non-limiting examples of isolated substances include (1) any non-naturally occurring substance, (2) any substance including, but not limited to, any host cell, enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated. The terms “isolated nucleic acid molecule”, “isolated polynucleotide”, and “isolated nucleic acid fragment” will be used interchangeably and refer to a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

The term “purified” as applied to nucleic acids or polypeptides generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

As used herein, the term “functional assay” refers to an assay that provides an indication of a protein's activity. In some embodiments, the term refers to assay systems in which a protein is analyzed for its ability to function in its usual capacity. For example, in the case of a protease, a functional assay involves determining the effectiveness of the protease to hydrolyze a proteinaceous substrate.

The terms “peptides”, “proteins” and “polypeptides are used interchangeably herein and refer to a polymer of amino acids joined together by peptide bonds. A “protein” or “polypeptide” comprises a polymeric sequence of amino acid residues. The single and 3-letter code for amino acids as defined in conformity with the IUPAC-IUB Joint Commission on Biochemical Nomenclature (JCBN) is used throughout this disclosure. The single letter X refers to any of the twenty amino acids. It is also understood that a polypeptide may be coded for by more than one nucleotide sequence due to the degeneracy of the genetic code. Mutations can be named by the one letter code for the parent amino acid, followed by a position number and then the one letter code for the variant amino acid. For example, mutating glycine (G) at position 87 to serine (S) is represented as “G087S” or “G87S”. When describing modifications, a position followed by amino acids listed in parentheses indicates a list of substitutions at that position by any of the listed amino acids. For example, 6(L,I) means position 6 can be substituted with a leucine or isoleucine. At times, in a sequence, a slash (/) is used to define substitutions, e.g. F/V, indicates that the particular position may have a phenylalanine or valine at that position.

A “prosequence” or “propeptide sequence” refers to an amino acid sequence between the signal peptide sequence and mature protease sequence that is necessary for the proper folding and secretion of the protease; they are sometimes referred to as intramolecular chaperones. Cleavage of the prosequence or propeptide sequence results in a mature active protease. Proteases are often expressed as pro-enzymes.

The terms “signal sequence” and “signal peptide” refer to a sequence of amino acid residues that may participate in the secretion or direct transport of the mature or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “mature” form of a protein, polypeptide, or peptide refers to the functional form of the protein, polypeptide, or enzyme without the signal peptide sequence and propeptide sequence.

The term “precursor” form of a protein or peptide refers to a mature form of the protein having a prosequence operably linked to the amino or carbonyl terminus of the protein. The precursor may also have a “signal” sequence operably linked to the amino terminus of the prosequence. The precursor may also have additional polypeptides that are involved in post-translational activity (e.g., polypeptides cleaved therefrom to leave the mature form of a protein or peptide).

The term “wild-type” in reference to an amino acid sequence or nucleic acid sequence indicates that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

As used herein with regard to amino acid residue positions, “corresponding to” or “corresponds to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related proteins or a reference protein.

The terms “derived from” and “obtained from” refer to not only a protein produced or producible by a strain of the organism in question, but also a protein encoded by a DNA sequence isolated from such strain and produced in a host organism containing such DNA sequence. Additionally, the term refers to a protein which is encoded by a DNA sequence of synthetic and/or cDNA origin and which has the identifying characteristics of the protein in question.

The term “reference”, with respect to a polypeptide described herein, refers to a naturally-occurring polypeptide that does not include a man-made substitution, insertion, or deletion at one or more amino acid positions, as well as a naturally-occurring or synthetic polypeptide that includes one or more man-made substitutions, insertions, or deletions at one or more amino acid positions. Similarly, the term “reference”, with respect to a polynucleotide, refers to a naturally-occurring polynucleotide that does not include a man-made substitution, insertion, or deletion of one or more nucleosides, as well as a naturally-occurring or synthetic polynucleotide that includes one or more man-made substitutions, insertions, or deletions at one or more nucleosides. For example, a polynucleotide encoding a wild-type or parental polypeptide is not limited to a naturally-occurring polynucleotide, and encompasses any polynucleotide encoding the wild-type or parental polypeptide.

The term “amino acid” refers to the basic chemical structural unit of a protein or polypeptide. The following abbreviations used herein to identify specific amino acids can be found in Table 2.

TABLE 2
One and Three Letter Amino Acid Abbreviations

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Thermostable serine acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid or as defined herein | Xaa | X |

It would be recognized by one of ordinary skill in the art that modifications of amino acid sequences disclosed herein can be made while retaining the function associated with the disclosed amino acid sequences. For example, it is well known in the art that alterations in a gene which result in the production of a chemically equivalent amino acid at a given site, but do not affect the functional properties of the encoded protein are common. For example, any particular amino acid in an amino acid sequence disclosed herein may be substituted for another functionally equivalent amino acid. For the purposes of this disclosure, substitutions are defined as exchanges within one of the following five groups:

1. Small aliphatic, nonpolar or slightly polar residues: Ala, Ser, Thr (Pro, Gly);

2. Polar, negatively charged residues and their amides: Asp, Asn, Glu, Gln;

3. Polar, positively charged residues: His, Arg, Lys;

4. Large aliphatic, nonpolar residues: Met, Leu, Ile, Val (Cys); and

5. Large aromatic residues: Phe, Tyr, and Trp.

Thus, a codon for the amino acid alanine, a hydrophobic amino acid, may be substituted by a codon encoding another less hydrophobic residue (such as glycine) or a more hydrophobic residue (such as valine, leucine, or isoleucine). Similarly, changes which result in substitution of one negatively charged residue for another (such as thermostable serine acid for glutamic acid) or one positively charged residue for another (such as lysine for arginine) can also be expected to produce a functionally equivalent product. In many cases, nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products.

The term “codon optimized”, as it refers to genes or coding regions of nucleic acid molecules for transformation of various hosts, refers to the alteration of codons in the gene or coding regions of the nucleic acid molecules to reflect the typical codon usage of the host organism without altering the polypeptide for which the DNA codes.

The term “gene” refers to a nucleic acid molecule that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different from that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “coding sequence” refers to a nucleotide sequence which codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding sites, and stem-loop structures.

The term “operably linked” refers to the association of nucleic acid sequences on a single nucleic acid molecule so that the function of one is affected by the other. For example, a promoter is operably linked with a coding sequence when it is capable of affecting the expression of that coding sequence, i.e., the coding sequence is under the transcriptional control of the promoter. Coding sequences can be operably linked to regulatory sequences in sense or antisense orientation.

The terms “regulatory sequence” or “control sequence” are used interchangeably herein and refer to a segment of a nucleotide sequence which is capable of increasing or decreasing expression of specific genes within an organism. Examples of regulatory sequences include, but are not limited to, promoters, signal sequence, operators and the like. As noted above, regulatory sequences can be operably linked in sense or antisense orientation to the coding sequence/gene of interest.

“Promoter” or “promoter sequences” refer to DNA sequences that define where transcription of a gene by RNA polymerase begins. Promoter sequences are typically located directly upstream or at the 5′ end of the transcription initiation site. Promoters may be derived in their entirety from a native or naturally occurring sequence, or be composed of different elements derived from different promoters found in nature, or even comprise synthetic DNA segments. It is understood by those skilled in the art that different promoters may direct the expression of a gene in different tissues or cell type or at different stages of development, or in response to different environmental or physiological conditions (“inducible promoters”).

The “3′ non-coding sequences” refer to DNA sequences located downstream of a coding sequence and include sequences encoding regulatory signals capable of affecting mRNA processing or gene expression, such as termination of transcription.

The term “transformation” as used herein refers to the transfer or introduction of a nucleic acid molecule into a host organism. The nucleic acid molecule may be introduced as a linear or circular form of DNA. The nucleic acid molecule may be a plasmid that replicates autonomously, or it may integrate into the genome of a production host. Production hosts containing the transformed nucleic acid are referred to as “transformed” or “recombinant” or “transgenic” organisms or “transformants”.

The term “recombinant” as used herein refers to an artificial combination of two otherwise separated segments of nucleic acid sequences, e.g., by chemical synthesis or by the manipulation of isolated segments of nucleic acids by genetic engineering techniques. For example, DNA in which one or more segments or genes have been inserted, either naturally or by laboratory manipulation, from a different molecule, from another part of the same molecule, or an artificial sequence, resulting in the introduction of a new sequence in a gene and subsequently in an organism. The terms “recombinant”, “transgenic”, “transformed”, “engineered” or “modified for exogenous gene expression” are used interchangeably herein.

The terms “recombinant construct”, “expression construct”, “recombinant expression construct” and “expression cassette” are used interchangeably herein. A recombinant construct comprises an artificial combination of nucleic acid fragments, e.g., regulatory and coding sequences that are not all found together in nature. For example, a construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J4:2411-2418; De Almeida et al., (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The terms “production host”, “host” and “host cell” are used interchangeably herein and refer to any organism, or cell thereof, whether human or non-human into which a recombinant construct can be stably or transiently introduced in order to express a gene. This term encompasses any progeny of a parent cell, which is not identical to the parent cell due to mutations that occur during propagation.

The term “percent identity” is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the number of matching nucleotides or amino acids between strings of such sequences. “Identity” and “similarity” can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Methods to determine identity and similarity are codified in publicly available computer programs.

As used herein, “% identity” or percent identity” or “PID” refers to protein sequence identity. Percent identity may be determined using standard techniques known in the art. Useful algorithms include the BLAST algorithms (See, Altschul et al., J Mol Biol, 215:403-410, 1990; and Karlin and Altschul, Proc Natl Acad Sci USA, 90:5873-5787, 1993). The BLAST program uses several search parameters, most of which are set to the default values. The NCBI BLAST algorithm finds the most relevant sequences in terms of biological similarity but is not recommended for query sequences of less than 20 residues (Altschul et al., Nucleic Acids Res, 25:3389-3402, 1997; and Schaffer et al., Nucleic Acids Res, 29:2994-3005, 2001). Exemplary default BLAST parameters for a nucleic acid sequence searches include: Neighboring words threshold=11; E-value cutoff=10; Scoring Matrix=NUC.3.1 (match=1, mismatch=−3); Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. A percent (%) amino acid sequence identity value is determined by the number of matching identical residues divided by the total number of residues of the “reference” sequence including any gaps created by the program for optimal/maximum alignment. BLAST algorithms refer to the “reference” sequence as the “query” sequence. As used herein, “homologous proteins” or “homologous proteases” refers to proteins that have distinct similarity in primary, secondary, and/or tertiary structure. Protein homology can refer to the similarity in linear amino acid sequence when proteins are aligned. Homologous search of protein sequences can be done using BLASTP and PSI-BLAST from NCBI BLAST with threshold (E-value cut-off) at 0.001. (Altschul S F, Madde T L, Shaffer A A, Zhang J, Zhang Z, Miller W, Lipman DJ. Gapped BLAST and PSI BLAST a new generation of protein database search programs. Nucleic Acids Res 1997 Set 1; 25(17):3389-402). Using this information, proteins sequences can be grouped. A phylogenetic tree can be built using the amino acid sequences.

Sequence alignments and percent identity calculations may be performed using the Megalign program of the LASERGENE bioinformatics computing suite (DNASTAR Inc., Madison, Wis.), the AlignX program of Vector NTI v. 7.0 (Informax, Inc., Bethesda, Md.), or the EMBOSS Open Software Suite (EMBL-EBI; Rice et al., Trends in Genetics 16, (6):276-277 (2000)). Multiple alignment of the sequences can be performed using the CLUSTAL method (such as CLUSTALW; for example version 1.83) of alignment (Higgins and Sharp, CABIOS, 5:151-153 (1989); Higgins et al., Nucleic Acids Res. 22:4673-4680 (1994); and Chenna et al., Nucleic Acids Res 31 (13):3497-500 (2003)), available from the European Molecular Biology Laboratory via the European Bioinformatics Institute) with the default parameters. Suitable parameters for CLUSTALW protein alignments include GAP Existence penalty=15, GAP extension=0.2, matrix=Gonnet (e.g., Gonnet250), protein ENDGAP=−1, protein GAPDIST=4, and KTUPLE=1. In one embodiment, a fast or slow alignment is used with the default settings where a slow alignment. Alternatively, the parameters using the CLUSTALW method (e.g., version 1.83) may be modified to also use KTUPLE=1, GAP PENALTY=10, GAP extension=1, matrix=BLOSUM (e.g., BLOSUM64), WINDOW=S, and TOP DIAGONALS SAVED=5.

The MUSCLE program (Robert C. Edgar. MUSCLE: multiple sequence alignment with high accuracy and high throughput Nucl. Acids Res. (2004) 32 (5): 1792-1797) is yet another example of a multiple sequence alignment algorithm.

Various polypeptide amino acid sequences and polynucleotide sequences are disclosed herein. Variants of these sequences that are at least about 70-85%, 85-90%, or 90%-95% identical to the sequences disclosed herein may be used in certain embodiments. Alternatively, a variant polypeptide sequence or polynucleotide sequence in certain embodiments can have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with a sequence disclosed herein. The variant amino acid sequence or polynucleotide sequence has the same function of the disclosed sequence, or at least about 85%, 86%, 87%, 88%, 89%, 90%, 91%, 92%, 93%, 94%, 95%, 96%, 97%, 98%, or 99% of the function of the disclosed sequence.

The term “variant”, with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

The terms “plasmid”, “vector” and “cassette” refer to an extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector containing a gene and having elements in addition to the gene that facilitates transformation of a particular host cell. The terms “expression cassette” and “expression vector are used interchangeably herein and refer to a specific vector containing a gene and having elements in addition to the gene that allow for expression of that gene in a host.

The term “expression”, as used herein, refers to the production of a functional end-product (e.g., an mRNA or a protein) in either precursor or mature form. Expression may also refer to translation of mRNA into a polypeptide.

Expression of a gene involves transcription of the gene and translation of the mRNA into a precursor or mature protein. “Mature” protein refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals. “Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms

The expression vector can be one of any number of vectors or cassettes useful for the transformation of suitable production hosts known in the art. Typically, the vector or cassette will include sequences directing transcription and translation of the relevant gene, a selectable marker, and sequences allowing autonomous replication or chromosomal integration. Suitable vectors generally include a region 5′ of the gene which harbors transcriptional initiation controls and a region 3′ of the DNA fragment which controls transcriptional termination. Both control regions can be derived from homologous genes to genes of a transformed production host cell and/or genes native to the production host, although such control regions need not be so derived.

Possible initiation control regions or promoters that can be included in the expression vector are numerous and familiar to those skilled in the art. Virtually any promoter capable of driving these genes is suitable, including but not limited to, CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, araB, tet, trp, lP_{L}, lP_{R}, T7, tac, and trc (useful for expression in Escherichia coli) as well as the amy, apr, npr promoters and various phage promoters useful for expression in Bacillus. In some embodiments, the promoter is a constitutive or inducible promoter. A “constitutive promoter” is a promoter that is active under most environmental and developmental conditions. An “inducible” or “repressible” promoter is a promoter that is active under environmental or developmental regulation. In some embodiments, promoters are inducible or repressible due to changes in environmental factors including but not limited to, carbon, nitrogen or other nutrient availability, temperature, pH, osmolarity, the presence of heavy metal(s), the concentration of inhibitor(s), stress, or a combination of the foregoing, as is known in the art. In some embodiments, the inducible or repressible promoters are inducible or repressible by metabolic factors, such as the level of certain carbon sources, the level of certain energy sources, the level of certain catabolites, or a combination of the foregoing as is known in the art. In one embodiment, the promoter is one that is native to the host cell. For example, when T. reesei is the host, the promoter is a native T. reesei promoter such as the cbh1 promoter which is deposited in GenBank under Accession Number D86235.

Suitable non-limiting examples of promoters include cbh1, cbh2, egl1, egl2, egl3, egl4, egl5, xyn1, and xyn2, repressible acid phosphatase gene (phoA) promoter of P. chrysogenus (see e.g., Graessle et al., (1997) Appl. Environ. Microbiol., 63:753-756), glucose repressible PCK1 promoter (see e.g., Leuker et al., (1997), Gene, 192:235-240), maltose inducible, glucose-repressible MET3 promoter (see Liu et al., (2006), Eukary. Cell, 5:638-649), pKi promoter and cpc1 promoter. Other examples of useful promoters include promoters from A. awamori and A. niger glucoamylase genes (see e.g., Nunberg et al., (1984) Mol. Cell Biol. 15 4:2306-2315 and Boel et al., (1984) EMBO J. 3:1581-1585). Also, the promoters of the T. reesei xln1 gene may be useful (see e.g., EPA 137280A1).

DNA fragments which control transcriptional termination may also be derived from various genes native to a preferred production host cell. In certain embodiments, the inclusion of a termination control region is optional. In certain embodiments, the expression vector includes a termination control region derived from the preferred host cell.

The expression vector can be included in the production host, particularly in the cells of microbial production hosts. The production host cells can be microbial hosts found within the fungal or bacterial families and which grow over a wide range of temperature, pH values, and solvent tolerances. For example, it is contemplated that any of bacteria, algae, and fungi such as filamentous fungi and yeast may suitably host the expression vector.

Inclusion of the expression vector in the production host cell may be used to express the protein of interest so that it may reside intracellularly, extracellularly, or a combination of both inside and outside the cell. Extracellular expression renders recovery of the desired protein from a fermentation product more facile than methods for recovery of protein produced by intracellular expression.

Certain embodiments of the present disclosure relate to an isolated polypeptide having serine protease activity, selected from:

a) a polypeptide comprising an amino acid sequence with at least 91% identity with the amino acid sequence of SEQ ID NO:22;

b) a polypeptide comprising an amino acid sequence with at least 94% identity with the amino acid sequence of SEQ ID NO:23;

c) a polypeptide comprising an amino acid sequence with at least 98% identity with the amino acid sequence of SEQ ID NO:24;

d) a polypeptide having an amino acid sequence with at least 80% identity with the amino acid sequence of SEQ ID NO:25.

In another embodiment, there is disclosed an isolated polypeptide having serine protease activity comprising a predicted precursor amino acid sequence selected from: SEQ ID NO:3; SEQ ID NO:6; SEQ ID NO:9; and SEQ ID NO:12.

In still another embodiment, there is disclosed an isolated polypeptide having serine protease activity and comprising a protease catalytic region, selected from the group consisting of:

a) an amino acid sequence with at least 96% identity with the amino acid sequence of SEQ ID NO:18

b) an amino acid sequence with at least 98% identity with the amino acid sequence of SEQ ID NO:19;

c) an amino acid sequence of SEQ ID NO:20;

d) an amino acid sequence with at least 91% identity with the amino acid sequence of SEQ ID NO:21

Other embodiments include a recombinant construct comprising a regulatory sequence functional in a production host operably linked to a nucleotide sequence encoding at least one polypeptide having serine protease activity selected:

a) a polypeptide comprising an amino acid sequence with at least 91% identity with the amino acid sequence of SEQ ID NO:22;

b) a polypeptide comprising an amino acid sequence with at least 94% identity with the amino acid sequence of SEQ ID NO:23;

c) a polypeptide comprising an amino acid sequence with at least 98% identity with the amino acid sequence of SEQ ID NO:24;

d) a polypeptide comprising an amino acid sequence with at least 80% identity with the amino acid sequence of SEQ ID NO:25.

The production host is selected from the group consisting of fungi, bacteria, and algae.

The production host can then be used in a method for producing at least one polypeptide having serine protease activity comprising:

(a) transforming a production host with the recombinant construct described herein; and

(b) culturing the production host of step (a) under conditions whereby at least one polypeptide having serine protease activity is produced.

According to this method, at least one polypeptide described herein is optionally recovered from the production host. In another aspect, a serine protease-containing culture supernatant is obtained by using any of the methods described herein.

Also described herein is a recombinant microbial production host for expressing at least one polypeptide described herein, said recombinant microbial production host comprising a recombinant construct described herein. In another embodiment, this recombinant microbial production host is selected from the group consisting of bacteria, fungi and algae.

Expression will be understood to include any step involved in producing at least one polypeptide described herein including, but not limited to, transcription, post-transcriptional modification, translation, post-translation modification and secretion.

Techniques for modifying nucleic acid sequences utilizing cloning methods are well known in the art.

A polynucleotide encoding a trypsin-like serine protease can be manipulated in a variety of ways to provide for expression of the polynucleotide in a Bacillus host cell. Manipulation of the polynucleotide sequence prior to its insertion into a nucleic acid construct or vector may be desirable or necessary depending on the nucleic acid construct or vector or the Bacillus host cell. The techniques for modifying nucleotide sequences utilizing cloning methods are well known in the art.

Regulatory sequences are defined above. They include all components, which are necessary or advantageous for the expression of a trypsin-like serine protease. Each control sequence may be native or foreign to the nucleotide sequence encoding the trypsin-like serine protease. Such regulatory sequences include, but are not limited to, a leader, a polyadenylation sequence, a propeptide sequence, a promoter, a signal sequence and a transcription terminator. Regulatory sequences may be provided with linkers for the purpose of introducing specific restriction sites facilitating ligation or the regulatory sequences with the coding region of the nucleotide sequence encoding a trypsin-like serine protease.

A nucleic acid construct comprising a polynucleotide encoding a trypsin-like serine protease may be operably linked to one or more control sequences capable of directing the expression of the coding sequence in a Bacillus host cell under conditions compatible with the control sequences.

Each control sequence may be native or foreign to the polynucleotide encoding a trypsin-like serine protease. Such control sequences include, but are not limited to, a leader, a promoter, a signal sequence, and a transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a trypsin-like serine protease.

The control sequence may be an appropriate promoter region, a nucleotide sequence that is recognized by a Bacillus host cell for expression of the polynucleotide encoding a trypsin-like serine protease. The promoter region contains transcription control sequences that mediate the expression of a trypsin-like serine protease. The promoter region may be any nucleotide sequence that shows transcriptional activity in the Bacillus host cell of choice and may be obtained from genes directing synthesis of extracellular or intracellular polypeptides having biological activity either homologous or heterologous to the Bacillus host cell.

The promoter region may comprise a single promoter or a combination of promoters. Where the promoter region comprises a combination of promoters, the promoters are preferably in tandem. A promoter of the promoter region can be any promoter that can initiate transcription of a polynucleotide encoding a polypeptide having biological activity in a Bacillus host cell of interest. The promoter may be native, foreign, or a combination thereof, to the nucleotide sequence encoding a polypeptide having biological activity. Such a promoter can be obtained from genes directing synthesis of extracellular or intracellular polypeptides having biological activity either homologous or heterologous to the Bacillus host cell.

Thus, in certain embodiments, the promoter region comprises a promoter obtained from a bacterial source. In other embodiments, the promoter region comprises a promoter obtained from a Gram positive or Gram negative bacterium. Gram positive bacteria include, but are not limited to, Bacillus, Streptococcus, Streptomyces, Staphylococcus, Enterococcus, Lactobacillus, Lactococcus, Clostridium, Geobacillus, and Oceanobacillus. Gram negative bacteria include, but are not limited to, E. coli, Pseudomonas, Salmonella, Campylobacter, Helicobacter, Flavobacterium, Fusobacterium, Ilyobacter, Neisseria, and Ureaplasma.

The promoter region may comprise a promoter obtained from a Bacillus strain (e.g., Bacillus agaradherens, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis; or from a Streptomyces strain (e.g., Streptomyces lividans or Streptomyces murinus).

Examples of suitable promoters for directing transcription of a polynucleotide encoding a polypeptide having biological activity in the methods of the present disclosure are the promoters obtained from the E. coli lac operon, Streptomyces coelicolor agarase gene (dagA), Bacillus lentus or Bacillus clausii alkaline protease gene (aprH), Bacillus licheniformis alkaline protease gene (subtilisin Carlsberg gene), Bacillus subtilis levansucrase gene (sacB), Bacillus subtilis alpha-amylase gene (amyE), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, Bacillus thuringiensis subsp. tenebfionis CryIIIA gene (cryIIIA) or portions thereof, prokaryotic beta-lactamase gene (Villa-Kamaroff et al., 1978, Proceedings of the National Academy of Sciences USA 75:3727-3731), and Bacillus megaterium xylA gene (Rygus and Hillen, 1992, J. Bacteriol. 174: 3049-3055; Kim et al., 1996, Gene 181: 71-76). Other examples are the promoter of the spoI bacterial phage promoter and the tac promoter (DeBoer et al., 1983, Proceedings of the National Academy of Sciences USA 80:21-25). Further promoters are described in “Useful proteins from recombinant bacteria” in Scientific American, 1980, 242:74-94; and in Sambrook, Fritsch, and Maniatis, 1989, Molecular Cloning, A Laboratory Manual, 2d edition, Cold Spring Harbor, N.Y.

The promoter region may comprise a promoter that is a “consensus” promoter having the sequence TTGACA for the “−35” region and TATAAT for the “−10” region. The consensus promoter may be obtained from any promoter that can function in a Bacillus host cell. The construction of a “consensus” promoter may be accomplished by site-directed mutagenesis using methods well known in the art to create a promoter that conforms more perfectly to the established consensus sequences for the “−10” and “−35” regions of the vegetative “sigma A-type” promoters for Bacillus subtilis (Voskuil et al., 1995, Molecular Microbiology 17: 271-279).

A control sequence may also be a suitable transcription terminator sequence, such as a sequence recognized by a Bacillus host cell to terminate transcription. The terminator sequence is operably linked to the 3′ terminus of the nucleotide sequence encoding a trypsin-like serine protease. Any terminator that is functional in the Bacillus host cell may be used.

The control sequence may also be a suitable leader sequence, a non-translated region of a mRNA that is important for translation by a Bacillus host cell. The leader sequence is operably linked to the 5′ terminus of the nucleotide sequence directing synthesis of the polypeptide having biological activity. Any leader sequence that is functional in a Bacillus host cell of choice may be used in the present invention.

The control sequence may also be a mRNA stabilizing sequence. The term “mRNA stabilizing sequence” is defined herein as a sequence located downstream of a promoter region and upstream of a coding sequence of a polynucleotide encoding a trypsin-like serine protease to which the promoter region is operably linked, such that all mRNAs synthesized from the promoter region may be processed to generate mRNA transcripts with a stabilizer sequence at the 5′ end of the transcripts. For example, the presence of such a stabilizer sequence at the 5′ end of the mRNA transcripts increases their half-life (Agaisse and Lereclus, 1994, supra, Hue et al., 1995, Journal of Bacteriology 177: 3465-3471). The mRNA processing/stabilizing sequence is complementary to the 3′ extremity of bacterial 16S ribosomal RNA. In certain embodiments, the mRNA processing/stabilizing sequence generates essentially single-size transcripts with a stabilizing sequence at the 5′ end of the transcripts. The mRNA processing/stabilizing sequence is preferably one, which is complementary to the 3′ extremity of a bacterial 16S ribosomal RNA. See, U.S. Pat. Nos. 6,255,076 and 5,955,310.

The nucleic acid construct can then be introduced into a Bacillus host cell using methods known in the art or those methods described herein for introducing and expressing a trypsin-like serine protease.

A nucleic acid construct comprising a DNA of interest encoding a protein of interest can also be constructed similarly as described above.

For obtaining secretion of the protein of interest of the introduced DNA, the control sequence may also comprise a signal peptide coding region, which codes for an amino acid sequence linked to the amino terminus of a polypeptide that can direct the expressed polypeptide into the cell's secretory pathway. The signal peptide coding region may be native to the polypeptide or may be obtained from foreign sources. The 5′ end of the coding sequence of the nucleotide sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to that portion of the coding sequence that encodes the secreted polypeptide. The foreign signal peptide coding region may be required where the coding sequence does not normally contain a signal peptide coding region. Alternatively, the foreign signal peptide coding region may simply replace the natural signal peptide coding region in order to obtain enhanced secretion of the polypeptide relative to the natural signal peptide coding region normally associated with the coding sequence. The signal peptide coding region may be obtained from an amylase or a protease gene from a Bacillus species. However, any signal peptide coding region capable of directing the expressed polypeptide into the secretory pathway of a Bacillus host cell of choice may be used in the present invention.

An effective signal peptide coding region for a Bacillus host cell, is the signal peptide coding region obtained from the maltogenic amylase gene from Bacillus NCIB 11837, the Bacillus stearothermophilus alpha-amylase gene, the Bacillus licheniformis subtilisin gene, the Bacillus licheniformis beta-lactamase gene, the Bacillus stearothermophilus neutral proteases genes (nprT, nprS, nprM), and the Bacillus subtilis prsA gene.

Thus, a polynucleotide construct comprising a nucleic acid encoding a trypsin-like serine protease construct comprising a nucleic acid encoding a polypeptide of interest (POI) can be constructed such that it is expressed by a host cell. Because of the known degeneracies in the genetic code, different polynucleotides encoding an identical amino acid sequence can be designed and made with routine skills in the art. For example, codon optimizations can be applied to optimize production in a particular host cell.

Nucleic acids encoding proteins of interest can be incorporated into a vector, wherein the vector can be transferred into a host cell using well-known transformation techniques, such as those disclosed herein.

The vector may be any vector that can be transformed into and replicated within a host cell. For example, a vector comprising a nucleic acid encoding a POI can be transformed and replicated in a bacterial host cell as a means of propagating and amplifying the vector. The vector also may be transformed into a Bacillus expression host of the disclosure, so that the protein encoding nucleic acid (e.g., an ORF) can be expressed as a functional protein.

A representative vector which can be modified with routine skill to comprise and express a nucleic acid encoding a POI is vector p2JM103BBI.

A polynucleotide encoding a trypsin-like serine protease or a POI can be operably linked to a suitable promoter, which allows transcription in the host cell. The promoter may be any nucleic acid sequence that shows transcriptional activity in the host cell of choice and may be derived from genes encoding proteins either homologous or heterologous to the host cell. Means of assessing promoter activity/strength are routine for the skilled artisan.

Examples of suitable promoters for directing the transcription of a polynucleotide sequence encoding comS1 polypeptide or a POI of the disclosure, especially in a bacterial host, include the promoter of the lac operon of E. coli, the Streptomyces coelicolor agarase gene dagA or celA promoters, the promoters of the Bacillus licheniformis alpha-amylase gene (amyL), the promoters of the Bacillus stearothermophilus maltogenic amylase gene (amyM), the promoters of the Bacillus amyloliquefaciens alpha-amylase (amyQ), the promoters of the Bacillus subtilis xylA and xylB genes, and the like.

A promoter for directing the transcription of a polynucleotide sequence encoding a POI can be a wild-type aprE promoter, a mutant aprE promoter or a consensus aprE promoter set forth in PCT International Publication No. WO2001/51643. In certain other embodiments, a promoter for directing the transcription of a polynucleotide sequence encoding a POI is a wild-type spoVG promoter, a mutant spoVG promoter, or a consensus spoVG promoter (Frisby and Zuber, 1991).

A promoter for directing the transcription of the polynucleotide sequence encoding a trypsin-like serine protease or a POI is a ribosomal promoter such as a ribosomal RNA promoter or a ribosomal protein promoter. The ribosomal RNA promoter can be a rrn promoter derived from B. subtilis, more particularly, the rrn promoter can be a rrnB, rrnI or rrnE ribosomal promoter from B. subtilis. In certain embodiments, the ribosomal RNA promoter is a P2 rrnI promoter from B. subtilis set forth in PCT International Publication No. WO2013/086219.

A suitable vector may further comprise a nucleic acid sequence enabling the vector to replicate in the host cell. Examples of such enabling sequences include the origins of replication of plasmids pUC19, pACYC177, pUB110, pE194, pAMB1, pIJ702, and the like.

A suitable vector may also comprise a selectable marker, e.g., a gene the product of which complements a defect in the isolated host cell, such as the dal genes from B. subtilis or B. licheniformis; or a gene that confers antibiotic resistance such as, e.g., ampicillin resistance, kanamycin resistance, chloramphenicol resistance, tetracycline resistance and the like.

A suitable expression vector typically includes components of a cloning vector, such as, for example, an element that permits autonomous replication of the vector in the selected host organism and one or more phenotypically detectable markers for selection purposes. Expression vectors typically also comprise control nucleotide sequences such as, for example, promoter, operator, ribosome binding site, translation initiation signal and optionally, a repressor gene, one or more activator genes sequences, or the like.

Additionally, a suitable expression vector may further comprise a sequence coding for an amino acid sequence capable of targeting the protein of interest to a host cell organelle such as a peroxisome, or to a particular host cell compartment. Such a targeting sequence may be, for example, the amino acid sequence “SKL”. For expression under the direction of control sequences, the nucleic acid sequence of the protein of interest can be operably linked to the control sequences in a suitable manner such that the expression takes place.

Protocols, such as described herein, used to ligate the DNA construct encoding a protein of interest, promoters, terminators and/or other elements, and to insert them into suitable vectors containing the information necessary for replication, are well known to persons skilled in the art.

An isolated cell, either comprising a polynucleotide construct or an expression vector, is advantageously used as a host cell in the recombinant production of a POI. The cell may be transformed with the DNA construct encoding the POI, conveniently by integrating the construct (in one or more copies) into the host chromosome. Integration is generally deemed an advantage, as the DNA sequence thus introduced is more likely to be stably maintained in the cell. Integration of the DNA constructs into the host chromosome may be performed applying conventional methods, for example, by homologous or heterologous recombination. For example, PCT International Publication No. WO2002/14490 describes methods of Bacillus transformation, transformants thereof and libraries thereof. Alternatively, the cell may be transformed with an expression vector as described above in connection with the different types of host cells.

It is, in other embodiments, advantageous to delete genes from expression hosts, where the gene deficiency can be cured by an expression vector. Known methods may be used to obtain a bacterial host cell having one or more inactivated genes. Gene inactivation may be accomplished by complete or partial deletion, by insertional inactivation or by any other means that renders a gene non-functional for its intended purpose, such that the gene is prevented from expression of a functional protein.

Techniques for transformation of bacteria and culturing the bacteria are standard and well known in the art. They can be used to transform the improved hosts of the present invention for the production of recombinant proteins of interest. Introduction of a DNA construct or vector into a host cell includes techniques such as transformation, electroporation, nuclear microinjection, transduction, transfection (e.g., lipofection mediated and DEAE-Dextrin mediated transfection), incubation with calcium phosphate DNA precipitate, high velocity bombardment with DNA-coated microprojectiles, gene gun or biolistic transformation and protoplast fusion, and the like. Transformation and expression methods for bacteria are also disclosed in Brigidi et al. (1990). A general transformation and expression protocol for protease deleted Bacillus strains is described in Ferrari et al. (U.S. Pat. No. 5,264,366).

Methods for transforming nucleic acids into filamentous fungi such as Aspergillus spp., e.g., A. oryzae or A. niger, H. grisea, H. insolens, and T. reesei. are well known in the art. A suitable procedure for transformation of Aspergillus host cells is described, for example, in EP238023. A suitable procedure for transformation of Trichoderma host cells is described, for example, in Steiger et al 2011, Appl. Environ. Microbiol. 77:114-121.

The choice of a production host can be any suitable microorganism such as bacteria, fungi and algae.

Typically, the choice will depend upon the gene encoding the trypsin-like serine protease and its source.

Introduction of a DNA construct or vector into a host cell includes techniques such as transformation; electroporation; nuclear microinjection; transduction; transfection, (e.g., lipofection mediated and DEAE-Dextrin mediated transfection); incubation with calcium phosphate DNA precipitate; high velocity bombardment with DNA-coated microprojectiles; and protoplast fusion. Basic texts disclosing the general methods that can be used include Sambrook et al., Molecular Cloning, A Laboratory Manual (2nd ed. 1989); Kriegler, Gene Transfer and Expression: A Laboratory Manual (1990); and Ausubel et al., eds., Current Protocols in Molecular Biology (1994)). The methods of transformation of the present invention may result in the stable integration of all or part of the transformation vector into the genome of a host cell, such as a filamentous fungal host cell. However, transformation resulting in the maintenance of a self-replicating extra-chromosomal transformation vector is also contemplated.

Many standard transfection methods can be used to produce bacterial and filamentous fungal (e.g. Aspergillus or Trichoderma) cell lines that express large quantities of the protease. Some of the published methods for the introduction of DNA constructs into cellulase-producing strains of Trichoderma include Lorito, Hayes, DiPietro and Harman, (1993) Curr. Genet. 24: 349-356; Goldman, VanMontagu and Herrera-Estrella, (1990) Curr. Genet. 17:169-174; and Penttila, Nevalainen, Ratto, Salminen and Knowles, (1987) Gene 6: 155-164, also see U.S. Pat. Nos. 6,022,725; 6,268,328 and Nevalainen et al., “The Molecular Biology of Trichoderma and its application to the Expression of Both Homologous and Heterologous Genes” in Molecular Industrial Mycology, Eds, Leong and Berka, Marcel Dekker Inc., NY (1992) pp 129-148; for Aspergillus include Yelton, Hamer and Timberlake, (1984) Proc. Natl. Acad. Sci. USA 81: 1470-1474, for Fusarium include Bajar, Podila and Kolattukudy, (1991) Proc. Natl. Acad. Sci. USA 88: 8202-8212, for Streptomyces include Hopwood et al., 1985, Genetic Manipulation of Streptomyces: Laboratory Manual, The John Innes Foundation, Norwich, UK and Fernandez-Abalos et al., Microbiol 149:1623-1632 (2003) and for Bacillus include Brigidi, DeRossi, Bertarini, Riccardi and Matteuzzi, (1990) FEMS Microbiol. Lett. 55: 135-138).

However, any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well-known methods for introducing cloned genomic DNA, cDNA, synthetic DNA or other foreign genetic material into a host cell (see, e.g., Sambrook et al., supra). Also of use is the Agrobacterium-mediated transfection method described in U.S. Pat. No. 6,255,115. It is only necessary that the particular genetic engineering procedure used be capable of successfully introducing at least one gene into the host cell capable of expressing the gene.

After the expression vector is introduced into the cells, the transfected or transformed cells are cultured under conditions favoring expression of genes under control of the promoter sequences.

The medium used to cultivate the cells may be any conventional medium suitable for growing the host cell and obtaining expression of an alpha-glucosidase polypeptide. Suitable media and media components are available from commercial suppliers or may be prepared according to published recipes (e.g., as described in catalogues of the American Type Culture Collection).

A thermostable serine polypeptide secreted from the host cells can be used, with minimal post-production processing, as a whole broth preparation.

Depending upon the host cell used post-transcriptional and/or post-translational modifications may be made. One non-limiting example of a post-transcriptional and/or post-translational modification is “clipping” or “truncation” of a polypeptide. For example, this may result in taking a trypsin-like serine protease from an inactive or substantially inactive state to an active state as in the case of a pro-peptide undergoing further post-translational processing to a mature peptide having the enzymatic activity. In another instance, this clipping may result in taking a mature thermostable serine protease polypeptide and further removing N or C-terminal amino acids to generate truncated forms of the thermostable serine protease that retain enzymatic activity.

Other examples of post-transcriptional or post-translational modifications include, but are not limited to, myristoylation, glycosylation, truncation, lipidation and tyrosine, serine or threonine phosphorylation. The skilled person will appreciate that the type of post-transcriptional or post-translational modifications that a protein may undergo may depend on the host organism in which the protein is expressed.

In some embodiments, the preparation of a spent whole fermentation broth of a recombinant microorganism can be achieved using any cultivation method known in the art resulting in the expression of a trypsin-like serine protease.

Fermentation may, therefore, be understood as comprising shake flask cultivation, small- or large-scale fermentation (including continuous, batch, fed-batch, or solid state fermentations) in laboratory or industrial fermenters performed in a suitable medium and under conditions allowing the alpha-glucosidase to be expressed or isolated. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

Host cells may be cultured under suitable conditions that allow expression of a trypsin-like serine protease. Expression of the enzymes may be constitutive such that they are continually produced, or inducible, requiring a stimulus to initiate expression. In the case of inducible expression, protein production can be initiated when required by, for example, addition of an inducer substance to the culture medium, for example dexamethasone or IPTG or sophorose.

Any of the fermentation methods well known in the art can suitably be used to ferment the transformed or the derivative fungal strain as described above. In some embodiments, fungal cells are grown under batch or continuous fermentation conditions.

A classical batch fermentation is a closed system, where the composition of the medium is set at the beginning of the fermentation, and the composition is not altered during the fermentation. At the beginning of the fermentation, the medium is inoculated with the desired organism(s). In other words, the entire fermentation process takes place without addition of any components to the fermentation system throughout.

Alternatively, a batch fermentation qualifies as a “batch” with respect to the addition of the carbon source. Moreover, attempts are often made to control factors such as pH and oxygen concentration throughout the fermentation process. Typically the metabolite and biomass compositions of the batch system change constantly up to the time the fermentation is stopped. Within batch cultures, cells progress through a static lag phase to a high growth log phase and finally to a stationary phase, where growth rate is diminished or halted. Left untreated, cells in the stationary phase would eventually die. In general, cells in log phase are responsible for the bulk of production of product. A suitable variation on the standard batch system is the “fed-batch fermentation” system. In this variation of a typical batch system, the substrate is added in increments as the fermentation progresses. Fed-batch systems are useful when it is known that catabolite repression would inhibit the metabolism of the cells, and/or where it is desirable to have limited amounts of substrates in the fermentation medium. Measurement of the actual substrate concentration in fed-batch systems is difficult and is therefore estimated on the basis of the changes of measurable factors, such as pH, dissolved oxygen and the partial pressure of waste gases, such as CO2. Batch and fed-batch fermentations are well known in the art.

Continuous fermentation is another known method of fermentation. It is an open system where a defined fermentation medium is added continuously to a bioreactor, and an equal amount of conditioned medium is removed simultaneously for processing. Continuous fermentation generally maintains the cultures at a constant density, where cells are maintained primarily in log phase growth. Continuous fermentation allows for the modulation of one or more factors that affect cell growth and/or product concentration. For example, a limiting nutrient, such as the carbon source or nitrogen source, can be maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology. Separation and concentration techniques are known in the art and conventional methods can be used to prepare a concentrated solution or broth comprising a trypsin-like serine protease polypeptide of the invention.

After fermentation, a fermentation broth is obtained, the microbial cells and various suspended solids, including residual raw fermentation materials, are removed by conventional separation techniques in order to obtain a trypsin-like serine protease solution. Filtration, centrifugation, microfiltration, rotary vacuum drum filtration, ultrafiltration, centrifugation followed by ultra-filtration, extraction, or chromatography, or the like, are generally used.

It may at times be desirable to concentrate a solution or broth comprising an alpha-glucosidase polypeptide to optimize recovery. Use of un-concentrated solutions or broth would typically increase incubation time in order to collect the enriched or purified enzyme precipitate.

The enzyme-containing solution can be concentrated using conventional concentration techniques until the desired enzyme level is obtained. Concentration of the enzyme containing solution may be achieved by any of the techniques discussed herein. Examples of methods of enrichment and purification include but are not limited to rotary vacuum filtration and/or ultrafiltration.

The trypsin-like serine protease-containing solution or broth may be concentrated until such time the enzyme activity of the concentrated a trypsin-like serine protease polypeptide-containing solution or broth is at a desired level.

Concentration may be performed using, e.g., a precipitation agent, such as a metal halide precipitation agent. Metal halide precipitation agents include but are not limited to alkali metal chlorides, alkali metal bromides and blends of two or more of these metal halides.

Exemplary metal halides include sodium chloride, potassium chloride, sodium bromide, potassium bromide and blends of two or more of these metal halides. The metal halide precipitation agent, sodium chloride, can also be used as a preservative. For production scale recovery, trypsin-like serine protease polypeptides can be enriched or partially purified as generally described above by removing cells via flocculation with polymers. Alternatively, the enzyme can be enriched or purified by microfiltration followed by concentration by ultrafiltration using available membranes and equipment. However, for some applications, the enzyme does not need to be enriched or purified, and whole broth culture can be lysed and used without further treatment. The enzyme can then be processed, for example, into granules.

Serine proteases may be isolated or purified in a variety of ways known to those skilled in the art depending on what other components are present in the sample. Standard purification methods include, but are not limited to, chromatography (e.g., ion exchange, affinity, hydrophobic, chromatofocusing, immunological and size exclusion), electrophoretic procedures (e.g., preparative isoelectric focusing), differential solubility (e.g., ammonium sulfate precipitation), extraction microfiltration, two phase separation. For example, the protein of interest may be purified using a standard anti-protein of interest antibody column. Ultrafiltration and diafiltration techniques, in conjunction with protein concentration, are also useful. For general guidance in suitable purification techniques, see Scopes, Protein Purification (1982). The degree of purification necessary will vary depending on the use of the protein of interest. In some instances, no purification will be necessary.

Assays for detecting and measuring the enzymatic activity of an enzyme, such as a trypsin-like serine protease polypeptide, are well known. Various assays for detecting and measuring activity of proteases (e.g., thermostable serine protease polypeptides), are also known to those of ordinary skill in the art. In particular, assays are available for measuring protease activity that are based on the release of acid-soluble peptides from casein or hemoglobin, measured as absorbance at 280 nm or colorimetrically using the Folin method, and hydrolysis of the dye-labeled azocasein, measured as absorbance at 440-450 nm

Other exemplary assays involve the solubilization of chromogenic substrates (See e.g., Ward, “Proteinases,” in Fogarty (ed.)., Microbial Enzymes and Biotechnology, Applied Science, London, [1983], pp. 251-317). A protease detection assay method using highly labeled fluorescein isothiocyanate (FITC) casein as the substrate, a modified version of the procedure described by Twining [Twining, S. S., (1984) “Fluorescein Isothiocyanate-Labeled Casein Assay for Proteolytic Enzymes” Anal. Biochem. 143:30-34] may also be used.

Other exemplary assays include, but are not limited to: cleavage of casein into trichloroacetic acid-soluble peptides containing tyrosine and tryptophan residues, followed by reaction with Folin-Ciocalteu reagent and colorimetric detection of products at 660 nm, cleavage of internally quenched FRET (Fluorescence Resonance Energy Transfer) peptide substrates followed by detection of product using a fluorometer. Fluorescence Resonance Energy Transfer (FRET) is the non-radiative transfer of energy from an excited fluorophore (or donor) to a suitable quencher (or acceptor) molecule. FRET is used in a variety of applications including the measurement of protease activity with substrates, in which the fluorophore is separated from the quencher by a short peptide sequence containing the enzyme cleavage site. Proteolysis of the peptide results in fluorescence as the fluorophore and quencher are separated. Numerous additional references known to those in the art provide suitable methods (See e.g., Wells et al., Nucleic Acids Res. 11:7911-7925 [1983]; Christianson et al., Anal. Biochem. 223:119-129 [1994]; and Hsia et al., Anal Biochem. 242:221-227 [1999]).

In still another aspect, there is disclosed a feed, feedstuff, feed additive composition, premix, food or grain product comprising at least one polypeptide having serine protease activity as described herein either alone or (a) in combination with at least one direct fed microbial or (b) in combination with at least one other enzyme or (c) in combination with at least one direct fed microbial and at least one other enzyme.

The at least one enzyme can be selected from, but is not limited to, enzymes such as, e.g., alpha-amylase, amyloglucosidase, phytase, pullulanase, beta-glucanase, cellulase, xylanase, etc..

Any of these enzymes can be used in an amount ranging from 0.5 to 500 micrograms/g feed or feedstock.

Alpha-amylases (alpha-1,4-glucan-4-glucanohydrolase, EC 3.2.1.1.) hydrolyze internal alpha-1,4-glucosidic linkages in starch, largely at random to produce smaller molecular weight dextrans. These polypeptides are used, inter alia, in starch processing and in alcohol production. Any alpha-amylases can be used, e.g., those described in U.S. Pat. Nos. 8,927,250 and 7,354,752.

Amyloglucosidase catalyzes the hydrolysis of terminal 1,4-linked alpha-D-glucose residues successively from the non-reducing ends of maltooligo- and polysaccharides with release of beta-D-glucose. Any amyloglucosidase can be used.

Phytase refers to a protein or polypeptide which is capable of catalyzing the hydrolysis of phytate to (1) myo-inositol and/or (2) mono-, di-, tri-, tetra-, and/or penta-phosphates thereof and (3) inorganic phosphate. For example, enzymes having catalytic activity as defined in Enzyme Commission EC number 3.1.3.8 or EC number 3.1.3.26. Any phytase can be used such as described in U.S. Pat. Nos. 8,144,046, 8,673,609, and 8,053,221.

Pullulanase (EC 3.2.1.41) is a specific kind of glucanase, an amylolytic exoenzyme that degrades pullan (a polysaccharide polymer consisting of maltotriose units, also known as alpha-1,4-; alpha-1,6-glucan. Thus, it is an example of a debranching enzyme. Pullulanase is also known as pullulan-6-glucanohydrolase. Pullulanases are generally secreted by a Bacillus species. For example, Bacillus deramificans (U.S. Pat. No. 5,817,498; 1998), Bacillus acidopullulyticus (European Patent No. 0 063 909) and Bacillus naganoensis (U.S. Pat. No. 5,055,403). Enzymes having pullulanase activity used commercially are produced, for example, from Bacillus species (trade name OPITMAX® 1-100 from DuPont-Genencor and Promozyme® D2 from Novozymes). Other examples of debranching enzymes include, but are not limited to, iso-amylase from Sulfolobus solfataricus, Pseudomonas sp. and thermostable pullulanase from Fervidobacterium nodosum (e.g., WO2010/76113). The iso-amylase from Pseudomonas sp. is available as purified enzyme from Megazyme International. Any pullulanase can be used.

Glucanases are enzymes that break down a glucan, a polysaccharide made several glucose sub-units. As they perform hydrolysis of the glycosidic bond, they are hydrolases.

Beta-glucanase enzymes (EC 3.2.1.4) digests fiber. It helps in the breakdown of plant walls (cellulose).

Cellulases are any of several enzymes produced by fungi, bacteria and protozoans that catalyze cellulolysis, the decomposition of cellulose and of some related polysaccharides. The name is also used for any naturally-occurring mixture or complex of various such enzymes, that act serially or synergistically to decompose cellulosic material. Any cellulases can be used.

Xylanase (EC 3.2.1.8) is the name given to a class of enzymes which degrade the linear polysaccharide beta-1,4-xylan into xylose, those breaking down hemicellulose, one of the major components of plant cell walls. Any xylanases can be used.

At least one DFM may comprise at least one viable microorganism such as a viable bacterial strain or a viable yeast or a viable fungi. Preferably, the DFM comprises at least one viable bacteria.

It is possible that the DFM may be a spore forming bacterial strain and hence the term DFM may be comprised of or contain spores, e.g. bacterial spores. Thus, the term “viable microorganism” as used herein may include microbial spores, such as endospores or conidia. Alternatively, the DFM in the feed additive composition described herein may not comprise of or may not contain microbial spores, e.g. endospores or conidia.

The microorganism may be a naturally-occurring microorganism or it may be a transformed microorganism.

A DFM as described herein may comprise microorganisms from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera and combinations thereof.

Preferably, the DFM comprises one or more bacterial strains selected from the following Bacillus spp: Bacillus subtilis, Bacillus cereus, Bacillus licheniformis, Bacillus pumilis and Bacillus amyloliquefaciens.

The genus “Bacillus”, as used herein, includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. gibsonii, B. pumilis and B. thuringiensis. It is recognized that the genus Bacillus continues to undergo taxonomical reorganization. Thus, it is intended that the genus include species that have been reclassified, including but not limited to such organisms as Bacillus stearothermophilus, which is now named “Geobacillus stearothermophilus”, or Bacillus polymyxa, which is now “Paenibacillus polymyxa” The production of resistant endospores under stressful environmental conditions is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

In another aspect, the DFM may be further combined with the following Lactococcus spp: Lactococcus cremoris and Lactococcus lactis and combinations thereof.

The DFM may be further combined with the following Lactobacillus spp: Lactobacillus buchneri, Lactobacillus acidophilus, Lactobacillus casei, Lactobacillus kefiri, Lactobacillus bifidus, Lactobacillus brevis, Lactobacillus helveticus, Lactobacillus paracasei, Lactobacillus rhamnosus, Lactobacillus salivarius, Lactobacillus curvatus, Lactobacillus bulgaricus, Lactobacillus sakei, Lactobacillus reuteri, Lactobacillus fermentum, Lactobacillus farciminis, Lactobacillus lactis, Lactobacillus delbreuckii, Lactobacillus plantarum, Lactobacillus paraplantarum, Lactobacillus farciminis, Lactobacillus rhamnosus, Lactobacillus crispatus, Lactobacillus gasseri, Lactobacillus johnsonii and Lactobacillus jensenii, and combinations of any thereof.

In still another aspect, the DFM may be further combined with the following Bifidobacteria spp: Bifidobacterium lactis, Bifidobacterium bifidium, Bifidobacterium longum, Bifidobacterium animalis, Bifidobacterium breve, Bifidobacterium infantis, Bifidobacterium catenulatum, Bifidobacterium pseudocatenulatum, Bifidobacterium adolescentis, and Bifidobacterium angulatum, and combinations of any thereof.

There can be mentioned bacteria of the following species: Bacillus subtilis, Bacillus licheniformis, Bacillus amyloliquefaciens, Bacillus pumilis, Enterococcus, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Bacillus subtilis, Propionibacterium thoenii, Lactobacillus farciminis, Lactobacillus rhamnosus, Megasphaera elsdenii, Clostridium butyricum, Bifidobacterium animalis ssp. animalis, Lactobacillus reuteri, Bacillus cereus, Lactobacillus salivarius ssp. Salivarius, Propionibacteria sp and combinations thereof.

A direct-fed microbial described herein comprising one or more bacterial strains may be of the same type (genus, species and strain) or may comprise a mixture of genera, species and/or strains.

Alternatively, a DFM may be combined with one or more of the products or the microorganisms contained in those products disclosed in WO2012110778, and summarized as follows:

Bacillus subtilis strain 2084 Accession No. NRR1 B-50013, Bacillus subtilis strain LSSAO1 Accession No. NRRL B-50104, and Bacillus subtilis strain 15A-P4 ATCC Accession No. PTA-6507 (from Enviva Pro®. (formerly known as Avicorr®); Bacillus subtilis Strain C3102 (from Calsporin®); Bacillus subtilis Strain PB6 (from Clostat®); Bacillus pumilis (8G-134); Enterococcus NCIMB 10415 (SF68) (from Cylactin®); Bacillus subtilis Strain C3102 (from Gallipro® & GalliproMax®); Bacillus licheniformis (from Gallipro®Tect®); Enterococcus and Pediococcus (from Poultry Star®); Lactobacillus, Bifidobacterium and/or Enterococcus from Protexin®); Bacillus subtilis strain QST 713 (from Proflora®); Bacillus amyloliquefaciens CECT-5940 (from Ecobiol® & Ecobiol® Plus); Enterococcus faecium SF68 (from Fortiflora®); Bacillus subtilis and Bacillus licheniformis (from BioPlus2B®); Lactic acid bacteria 7 Enterococcus faecium (from Lactiferm®); Bacillus strain (from CSI®); Saccharomyces cerevisiae (from Yea-Sacc®); Enterococcus (from Biomin IMB52®); Pediococcus acidilactici, Enterococcus, Bifidobacterium animalis ssp. animalis, Lactobacillus reuteri, Lactobacillus salivarius ssp. salivarius (from Biomin C5®); Lactobacillus farciminis (from Biacton®); Enterococcus (from Oralin E1707®); Enterococcus (2 strains), Lactococcus lactis DSM 1103(from Probios-pioneer PDFM®); Lactobacillus rhamnosus and Lactobacillus farciminis (from Sorbiflore®); Bacillus subtilis (from Animavit®); Enterococcus (from Bonvital®); Saccharomyces cerevisiae (from Levucell SB 20®); Saccharomyces cerevisiae (from Levucell SC 0 & SC10® ME); Pediococcus acidilacti (from Bactocell); Saccharomyces cerevisiae (from ActiSaf® (formerly BioSaf®)); Saccharomyces cerevisiae NCYC Sc47 (from Actisaf® SC47); Clostridium butyricum (from Miya-Gold®); Enterococcus (from Fecinor and Fecinor Plus®); Saccharomyces cerevisiae NCYC R-625 (from InteSwine®); Saccharomyces cerevisia (from BioSprint®); Enterococcus and Lactobacillus rhamnosus (from Provita®); Bacillus subtilis and Aspergillus oryzae (from PepSoyGen-C®); Bacillus cereus (from Toyocerin®); Bacillus cereus var. toyoi NCIMB 40112/CNCM 1-1012 (from TOYOCERIN®), or other DFMs such as Bacillus licheniformis and Bacillus subtilis (from BioPlus® YC) and Bacillus subtilis (from GalliPro®).

The DFM may be combined with Enviva® PRO which is commercially available from Danisco A/S. Enviva Pro® is a combination of Bacillus strain 2084 Accession No. NRR1 B-50013, Bacillus strain LSSAO1 Accession No. NRRL B-50104 and Bacillus strain 15A-P4 ATCC Accession No. PTA-6507 (as taught in U.S. Pat. No. 7,754,469 B—incorporated herein by reference).

It is also possible to combine the DFM described herein with a yeast from the genera: Saccharomyces spp.

Preferably, the DFM described herein comprises microorganisms which are generally recognized as safe (GRAS) and, preferably are GRAS-approved.

A person of ordinary skill in the art will readily be aware of specific species and/or strains of microorganisms from within the genera described herein which are used in the food and/or agricultural industries and which are generally considered suitable for animal consumption.

In some embodiments, it is important that the DFM be heat tolerant, i.e. is thermotolerant. This is particularly the case when the feed is pelleted. Therefore, in another embodiment, the DFM may be a thermotolerant microorganism, such as a thermotolerant bacteria, for example Bacillus spp.

In other aspects, it may be desirable that the DFM comprises a spore producing bacteria, such as Bacilli, e.g. Bacillus spp. Bacilli are able to form stable endospores when conditions for growth are unfavorable and are very resistant to heat, pH, moisture and disinfectants.

The DFM described herein may decrease or prevent intestinal establishment of pathogenic microorganism (such as Clostridium perfringens and/or E. coli and/or Salmonella spp and/or Campylobacter spp.). In other words, the DFM may be antipathogenic. The term “antipathogenic” as used herein means the DFM counters an effect (negative effect) of a pathogen.

As described above, the DFM may be any suitable DFM. For example, the following assay “DFM ASSAY” may be used to determine the suitability of a microorganism to be a DFM. The DFM assay as used herein is explained in more detail in US2009/0280090. For avoidance of doubt, the DFM selected as an inhibitory strain (or an antipathogenic DFM) in accordance with the “DFM ASSAY” taught herein is a suitable DFM for use in accordance with the present disclosure, i.e. in the feed additive composition according to the present disclosure.

Tubes were seeded each with a representative pathogen (e.g., bacteria) from a representative cluster.

Supernatant from a potential DFM, grown aerobically or anaerobically, is added to the seeded tubes (except for the control to which no supernatant is added) and incubated. After incubation, the optical density (OD) of the control and supernatant treated tubes was measured for each pathogen.

Colonies of (potential DFM) strains that produced a lowered OD compared with the control (which did not contain any supernatant) can then be classified as an inhibitory strain (or an antipathogenic DFM). Thus, The DFM assay as used herein is explained in more detail in US2009/0280090.

Preferably, a representative pathogen used in this DFM assay can be one (or more) of the following: Clostridium, such as Clostridium perfringens and/or Clostridium difficile, and/or E. coli and/or Salmonella spp and/or Campylobacter spp. In one preferred embodiment the assay is conducted with one or more of Clostridium perfringens and/or Clostridium difficile and/or E. coli, preferably Clostridium perfringens and/or Clostridium difficile, more preferably Clostridium perfringens.

Antipathogenic DFMs include one or more of the following bacteria and are described in WO2013029013.:

Bacillus subtilis strain 3BP5 Accession No. NRRL B-50510,

Bacillus subtilis strain 918 ATCC Accession No. NRRL B-50508, and

Bacillus subtilis strain 1013 ATCC Accession No. NRRL B-50509.

DFMs may be prepared as culture(s) and carrier(s) (where used) and can be added to a ribbon or paddle mixer and mixed for about 15 minutes, although the timing can be increased or decreased. The components are blended such that a uniform mixture of the cultures and carriers result. The final product is preferably a dry, flowable powder. The DFM(s) comprising one or more bacterial strains can then be added to animal feed or a feed premix, added to an animal's water, or administered in other ways known in the art (preferably simultaneously with the enzymes described herein.

Inclusion of the individual strains in the DFM mixture can be in proportions varying from 1% to 99% and, preferably, from 25% to 75%

Suitable dosages of the DFM in animal feed may range from about 1×10³ CFU/g feed to about 1×10¹⁰ CFU/g feed, suitably between about 1×10⁴ CFU/g feed to about 1×10⁸ CFU/g feed, suitably between about 7.5×10⁴ CFU/g feed to about 1×10⁷ CFU/g feed.

In another aspect, the DFM may be dosed in feedstuff at more than about 1×10³ CFU/g feed, suitably more than about 1×10⁴ CFU/g feed, suitably more than about 5×10⁴ CFU/g feed, or suitably more than about 1×10⁵ CFU/g feed.

The DFM may be dosed in a feed additive composition from about 1×10³ CFU/g composition to about 1×10¹³ CFU/g composition, preferably 1×10⁵ CFU/g composition to about 1×10¹³ CFU/g composition, more preferably between about 1×10⁶ CFU/g composition to about 1×10¹² CFU/g composition, and most preferably between about 3.75×10⁷ CFU/g composition to about 1×10¹¹ CFU/g composition. In another aspect, the DFM may be dosed in a feed additive composition at more than about 1×10⁵ CFU/g composition, preferably more than about 1×10⁶ CFU/g composition, and most preferably more than about 3.75×10⁷ CFU/g composition. In one embodiment the DFM is dosed in the feed additive composition at more than about 2×10⁵ CFU/g composition, suitably more than about 2×10⁶ CFU/g composition, suitably more than about 3.75×10⁷ CFU/g composition.

A feed additive composition for use in animal feed may comprise at least one polypeptide having serine protease activity as described herein, used either alone or (a) in combination with at least one direct fed microbial or (b) in combination with at least one other enzyme or (c) in combination with at least one direct fed microbial and at least one other enzyme, and (d) at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabisulfite, methyl paraben and propyl paraben.

In still another aspect, there is disclosed a granulated feed additive composition for use in animal feed comprising a at least one polypeptide having serine protease activity as described herein, used either alone or in combination with at least one direct fed microbial or in combination with at least one other enzyme or in combination with at least one direct fed microbial and at least one other enzyme, wherein the granulated feed additive composition comprises particles produced by a process selected from the group consisting of high shear granulation, drum granulation, extrusion, spheronization, fluidized bed agglomeration, fluidized bed spray coating, spray drying, freeze drying, prilling, spray chilling, spinning disk atomization, coacervation, tableting, or any combination of the above processes.

Furthermore, the particles of the granulated feed additive composition can have a mean diameter of greater than 50 microns and less than 2000 microns

The feed additive composition can be a liquid form and the liquid form can also be said suitable for spray-drying on a feed pellet.

Animal feeds may include plant material such as corn, wheat, sorghum, soybean, canola, sunflower or mixtures of any of these plant materials or plant protein sources for poultry, pigs, ruminants, aquaculture and pets. It is contemplated that animal performance parameters, such as growth, feed intake and feed efficiency, but also improved uniformity, reduced ammonia concentration in the animal house and consequently improved welfare and health status of the animals will be improved. More specifically, as used herein, “animal performance” may be determined by the feed efficiency and/or weight gain of the animal and/or by the feed conversion ratio and/or by the digestibility of a nutrient in a feed (e.g. amino acid digestibility) and/or digestible energy or metabolizable energy in a feed and/or by nitrogen retention and/or by animals ability to avoid the negative effects of necrotic enteritis and/or by the immune response of the subject.

Preferably “animal performance” is determined by feed efficiency and/or weight gain of the animal and/or by the feed conversion ratio.

By “improved animal performance” it is meant that there is increased feed efficiency, and/or increased weight gain and/or reduced feed conversion ratio and/or improved digestibility of nutrients or energy in a feed and/or by improved nitrogen retention and/or by improved ability to avoid the negative effects of necrotic enteritis and/or by an improved immune response in the subject resulting from the use of feed additive composition of the present invention in feed in comparison to feed which does not comprise said feed additive composition.

Preferably, by “improved animal performance” it is meant that there is increased feed efficiency and/or increased weight gain and/or reduced feed conversion ratio. As used herein, the term “feed efficiency” refers to the amount of weight gain in an animal that occurs when the animal is fed ad-libitum or a specified amount of food during a period of time.

By “increased feed efficiency” it is meant that the use of a feed additive composition according the present invention in feed results in an increased weight gain per unit of feed intake compared with an animal fed without said feed additive composition being present.

As used herein, the term “feed conversion ratio” refers to the amount of feed fed to an animal to increase the weight of the animal by a specified amount.

An improved feed conversion ratio means a lower feed conversion ratio.

By “lower feed conversion ratio” or “improved feed conversion ratio” it is meant that the use of a feed additive composition in feed results in a lower amount of feed being required to be fed to an animal to increase the weight of the animal by a specified amount compared to the amount of feed required to increase the weight of the animal by the same amount when the feed does not comprise said feed additive composition.

Nutrient digestibility as used herein means the fraction of a nutrient that disappears from the gastro-intestinal tract or a specified segment of the gastro-intestinal tract, e.g. the small intestine. Nutrient digestibility may be measured as the difference between what is administered to the subject and what comes out in the faeces of the subject, or between what is administered to the subject and what remains in the digesta on a specified segment of the gastro intestinal tract, e.g. the ileum.

Nutrient digestibility as used herein may be measured by the difference between the intake of a nutrient and the excreted nutrient by means of the total collection of excreta during a period of time; or with the use of an inert marker that is not absorbed by the animal, and allows the researcher calculating the amount of nutrient that disappeared in the entire gastro-intestinal tract or a segment of the gastro-intestinal tract. Such an inert marker may be titanium dioxide, chromic oxide or acid insoluble ash. Digestibility may be expressed as a percentage of the nutrient in the feed, or as mass units of digestible nutrient per mass units of nutrient in the feed.

Nutrient digestibility as used herein encompasses starch digestibility, fat digestibility, protein digestibility, and amino acid digestibility.

Energy digestibility as used herein means the gross energy of the feed consumed minus the gross energy of the faeces or the gross energy of the feed consumed minus the gross energy of the remaining digesta on a specified segment of the gastro-intestinal tract of the animal, e.g. the ileum. Metabolizable energy as used herein refers to apparent metabolizable energy and means the gross energy of the feed consumed minus the gross energy contained in the faeces, urine, and gaseous products of digestion. Energy digestibility and metabolizable energy may be measured as the difference between the intake of gross energy and the gross energy excreted in the faeces or the digesta present in specified segment of the gastro-intestinal tract using the same methods to measure the digestibility of nutrients, with appropriate corrections for nitrogen excretion to calculate metabolizable energy of feed.

In some embodiments, the compositions described herein can improve the digestibility or utilization of dietary hemicellulose or fibre in a subject. In some embodiments, the subject is a pig.

Nitrogen retention as used herein means as subject's ability to retain nitrogen from the diet as body mass. A negative nitrogen balance occurs when the excretion of nitrogen exceeds the daily intake and is often seen when the muscle is being lost. A positive nitrogen balance is often associated with muscle growth, particularly in growing animals.

Nitrogen retention may be measured as the difference between the intake of nitrogen and the excreted nitrogen by means of the total collection of excreta and urine during a period of time. It is understood that excreted nitrogen includes undigested protein from the feed, endogenous proteinaceous secretions, microbial protein, and urinary nitrogen.

The term survival as used herein means the number of subject remaining alive. The term “improved survival” may be another way of saying “reduced mortality”.

The term carcass yield as used herein means the amount of carcass as a proportion of the live body weight, after a commercial or experimental process of slaughter. The term carcass means the body of an animal that has been slaughtered for food, with the head, entrails, part of the limbs, and feathers or skin removed. The term meat yield as used herein means the amount of edible meat as a proportion of the live body weight, or the amount of a specified meat cut as a proportion of the live body weight.

An “increased weight gain” refers to an animal having increased body weight on being fed feed comprising a feed additive composition compared with an animal being fed a feed without said feed additive composition being present.

The term “animal” as used herein includes all non-ruminant and ruminant animals. In a particular embodiment, the animal is a non-ruminant animal, such as a horse and a mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment the animal is a ruminant animal including, but not limited to, cattle, young calves, goats, sheep, giraffes, bison, moose, elk, yaks, water buffalo, deer, camels, alpacas, llamas, antelope, pronghorn and nilgai.

In the present context, it is intended that the term “pet food” is understood to mean a food for a household animal such as, but not limited to, dogs, cats, gerbils, hamsters, chinchillas, fancy rats, guinea pigs; avian pets, such as canaries, parakeets, and parrots; reptile pets, such as turtles, lizards and snakes; and aquatic pets, such as tropical fish and frogs.

The terms “animal feed composition,” “feed”, “feedstuff” and “fodder” are used interchangeably and can comprise one or more feed materials selected from the group comprising a) cereals, such as small grains (e.g., wheat, barley, rye, oats and combinations thereof) and/or large grains such as maize or sorghum; b) by products from cereals, such as corn gluten meal, Distillers Dried Grains with Solubles (DDGS) (particularly corn based Distillers Dried Grains with Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; and/or e) minerals and vitamins.

Trypsin-like serine proteases described herein or a feed additive composition may be used as, or in the preparation of, a feed. The terms “feed additive composition” and “enzyme composition” are used interchangeably herein.

The feed may be in the form of a solution or as a solid or as a semi-solid depending on the use and/or the mode of application and/or the mode of administration.

When used as, or in the preparation of, a feed, such as functional feed, the enzyme or feed additive composition described herein may be used in conjunction with one or more of: a nutritionally acceptable carrier, a nutritionally acceptable diluent, a nutritionally acceptable excipient, a nutritionally acceptable adjuvant, a nutritionally active ingredient. For example, there be mentioned at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabisulfite, methyl paraben and propyl paraben.

In a preferred embodiment the enzyme or feed additive composition of the present invention is admixed with a feed component to form a feedstuff. The term “feed component” as used herein means all or part of the feedstuff. Part of the feedstuff may mean one constituent of the feedstuff or more than one constituent of the feedstuff, e.g. 2 or 3 or 4 or more. In one embodiment the term “feed component” encompasses a premix or premix constituents. Preferably, the feed may be a fodder, or a premix thereof, a compound feed, or a premix thereof. A feed additive composition may be admixed with a compound feed, a compound feed component or to a premix of a compound feed or to a fodder, a fodder component, or a premix of a fodder.

Any feedstuff described herein may comprise one or more feed materials selected from the group comprising a) cereals, such as small grains (e.g., wheat, barley, rye, oats, triticale and combinations thereof) and/or large grains such as maize or sorghum; b) by products from cereals, such as corn gluten meal, wet-cake (particularly corn based wet-cake), Distillers Dried Grains (DDG) (particularly corn based Distillers Dried Grains (cDDG)), Distillers Dried Grains with Solubles (DDGS) (particularly corn based Distillers Dried Grains with Solubles (cDDGS)), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; e) minerals and vitamins.

The term “fodder” as used herein means any food which is provided to an animal (rather than the animal having to forage for it themselves). Fodder encompasses plants that have been cut. Furthermore, fodder includes silage, compressed and pelleted feeds, oils and mixed rations, and also sprouted grains and legumes.

Fodder may be obtained from one or more of the plants selected from: corn (maize), alfalfa (Lucerne), barley, birdsfoot trefoil, brassicas, Chau moellier, kale, rapeseed (canola), rutabaga (swede), turnip, clover, alsike clover, red clover, subterranean clover, white clover, fescue, brome, millet, oats, sorghum, soybeans, trees (pollard tree shoots for tree-hay), wheat, and legumes.

The term “compound feed” means a commercial feed in the form of a meal, a pellet, nuts, cake or a crumble. Compound feeds may be blended from various raw materials and additives. These blends are formulated according to the specific requirements of the target animal.

Compound feeds can be complete feeds that provide all the daily required nutrients, concentrates that provide a part of the ration (protein, energy) or supplements that only provide additional micronutrients, such as minerals and vitamins.

The main ingredients used in compound feed are the feed grains, which include corn, wheat, canola meal, rapeseed meal, lupin, soybeans, sorghum, oats, and barley.

Suitably a premix as referred to herein may be a composition composed of microingredients such as vitamins, minerals, chemical preservatives, antibiotics, fermentation products, and other essential ingredients. Premixes are usually compositions suitable for blending into commercial rations.

In one embodiment the feedstuff comprises or consists of corn, DDGS (such as cDDGS), wheat, wheat bran or any combination thereof.

In one embodiment the feed component may be corn, DDGS (e.g. cDDGS), wheat, wheat bran or a combination thereof. In one embodiment the feedstuff comprises or consists of corn, DDGS (such as cDDGS) or a combination thereof.

A feedstuff described herein may contain at least 30%, at least 40%, at least 50% or at least 60% by weight corn and soybean meal or corn and full fat soy, or wheat meal or sunflower meal.

For example, a feedstuff may contain between about 5 to about 40% corn DDGS. For poultry, the feedstuff on average may contain between about 7 to 15% corn DDGS. For swine (pigs), the feedstuff may contain on average 5 to 40% corn DDGS. It may also contain corn as a single grain, in which case the feedstuff may comprise between about 35% to about 80% corn.

In feedstuffs comprising mixed grains, e.g. comprising corn and wheat for example, the feedstuff may comprise at least 10% corn.

In addition or in the alternative, a feedstuff also may comprise at least one high fibre feed material and/or at least one by-product of the at least one high fibre feed material to provide a high fibre feedstuff. Examples of high fibre feed materials include: wheat, barley, rye, oats, by products from cereals, such as corn gluten meal, corn gluten feed, wet-cake, Distillers Dried Grains (DDG), Distillers Dried Grains with Solubles (DDGS), wheat bran, wheat middlings, wheat shorts, rice bran, rice hulls, oat hulls, palm kernel, and citrus pulp. Some protein sources may also be regarded as high fibre: protein obtained from sources such as sunflower, lupin, fava beans and cotton. In one aspect, the feedstuff as described herein comprises at least one high fibre material and/or at least one by-product of the at least one high fibre feed material selected from the group consisting of Distillers Dried Grains with Solubles (DDGS), particularly cDDGS, wet-cake, Distillers Dried Grains (DDG), particularly cDDG, wheat bran, and wheat for example. In one embodiment the feedstuff of the present invention comprises at least one high fibre material and/or at least one by-product of the at least one high fibre feed material selected from the group consisting of Distillers Dried Grains with Solubles (DDGS), particularly cDDGS, wheat bran, and wheat for example.

The feed may be one or more of the following: a compound feed and premix, including pellets, nuts or (cattle) cake; a crop or crop residue: corn, soybeans, sorghum, oats, barley copra, straw, chaff, sugar beet waste; fish meal; meat and bone meal; molasses; oil cake and press cake; oligosaccharides; conserved forage plants: silage; seaweed; seeds and grains, either whole or prepared by crushing, milling etc.; sprouted grains and legumes; yeast extract.

The term “feed” as used herein encompasses in some embodiments pet food. A pet food is plant or animal material intended for consumption by pets, such as dog food or cat food. Pet food, such as dog and cat food, may be either in a dry form, such as kibble for dogs, or wet canned form. Cat food may contain the amino acid taurine.

Animal feed can also include a fish food. A fish food normally contains macro nutrients, trace elements and vitamins necessary to keep captive fish in good health. Fish food may be in the form of a flake, pellet or tablet. Pelleted forms, some of which sink rapidly, are often used for larger fish or bottom feeding species. Some fish foods also contain additives, such as beta carotene or sex hormones, to artificially enhance the color of ornamental fish.

In still another aspect, animal feed encompasses bird food. Bird food includes food that is used both in birdfeeders and to feed pet birds. Typically bird food comprises of a variety of seeds, but may also encompass suet (beef or mutton fat).

As used herein the term “contacted” refers to the indirect or direct application of a trypsin-like serine protease enzyme (or composition comprising the thermostable serine protease) to a product (e.g. the feed). Examples of application methods which may be used, include, but are not limited to, treating the product in a material comprising the feed additive composition, direct application by mixing the feed additive composition with the product, spraying the feed additive composition onto the product surface or dipping the product into a preparation of the feed additive composition. In one embodiment the feed additive composition of the present invention is preferably admixed with the product (e.g. feedstuff). Alternatively, the feed additive composition may be included in the emulsion or raw ingredients of a feedstuff. For some applications, it is important that the composition is made available on or to the surface of a product to be affected/treated. This allows the composition to impart a performance benefit.

In some aspects, the thermostable serine proteases described are used for the pretreatment of food or feed. For example, the feed having 10-300% moisture is mixed and incubated with the proteases at 5-80° C., preferably at 25-50° C., more preferably between 30-45° C. for 1 min to 72 hours under aerobic conditions or 1 day to 2 months under anaerobic conditions. The pre-treated material can be fed directly to the animals (so called liquid feeding). The pre-treated material can also be steam pelleted at elevated temperatures of 60-120° C. The proteases can be impregnated to feed or food material by a vacuum coater.

Trypsin-like serine proteases (or composition comprising the thermostable serine proteases) may be applied to intersperse, coat and/or impregnate a product (e.g. feedstuff or raw ingredients of a feedstuff) with a controlled amount of said enzyme.

Preferably, the feed additive composition will be thermally stable to heat treatment up to about 70° C.; up to about 85° C.; or up to about 95° C. The heat treatment may be performed for up to about 1 minute; up to about 5 minutes; up to about 10 minutes; up to about 30 minutes; up to about 60 minutes. The term thermally stable means that at least about 75% of the enzyme components and/or DFM that were present/active in the additive before heating to the specified temperature are still present/active after it cools to room temperature. Preferably, at least about 80% of the protease component and/or DFM comprising one or more bacterial strains that were present and active in the additive before heating to the specified temperature are still present and active after it cools to room temperature. In a particularly preferred embodiment the feed additive composition is homogenized to produce a powder.

Alternatively, the feed additive composition is formulated to granules as described in WO2007/044968 (referred to as TPT granules) incorporated herein by reference.

In another preferred embodiment when the feed additive composition is formulated into granules the granules comprise a hydrated barrier salt coated over the protein core. The advantage of such salt coating is improved thermo-tolerance, improved storage stability and protection against other feed additives otherwise having adverse effect on the at least one protease and/or DFM comprising one or more bacterial strains. Preferably, the salt used for the salt coating has a water activity greater than 0.25 or constant humidity greater than 60% at 20° C. Preferably, the salt coating comprises a Na₂SO₄.

The method of preparing a feed additive composition may also comprise the further step of pelleting the powder. The powder may be mixed with other components known in the art. The powder, or mixture comprising the powder, may be forced through a die and the resulting strands are cut into suitable pellets of variable length.

A method of preparing trypsin-like serine proteases (or composition comprising the thermostable serine proteases) may also comprise the further step of pelleting the powder. The powder may be mixed with other components known in the art. The powder, or mixture comprising the powder, may be forced through a die and the resulting strands are cut into suitable pellets of variable length.

Optionally, the pelleting step may include a steam treatment, or conditioning stage, prior to formation of the pellets. The mixture comprising the powder may be placed in a conditioner, e.g. a mixer with steam injection. The mixture is heated in the conditioner up to a specified temperature, such as from 60-100° C., typical temperatures would be 70° C., 80° C., 85° C., 90° C. or 95° C. The residence time can be variable from seconds to minutes and even hours. Such as 5 seconds, 10 seconds, 15 seconds, 30 seconds, 1 minutes 2 minutes., 5 minutes, 10 minutes, 15 minutes, 30 minutes and 1 hour. It will be understood that the thermostable serine proteases (or composition comprising the thermostable serine proteases) described herein are suitable for addition to any appropriate feed material.

It will be understood by the skilled person that different animals require different feedstuffs, and even the same animal may require different feedstuffs, depending upon the purpose for which the animal is reared.

Optionally, the feedstuff may also contain additional minerals such as, for example, calcium and/or additional vitamins. In some embodiments, the feedstuff is a corn soybean meal mix.

Feedstuff is typically produced in feed mills in which raw materials are first ground to a suitable particle size and then mixed with appropriate additives. The feedstuff may then be produced as a mash or pellets; the later typically involves a method by which the temperature is raised to a target level and then the feed is passed through a die to produce pellets of a particular size. The pellets are allowed to cool. Subsequently liquid additives such as fat and enzyme may be added. Production of feedstuff may also involve an additional step that includes extrusion or expansion prior to pelleting, in particular by suitable techniques that may include at least the use of steam.

The feedstuff may be a feedstuff for a monogastric animal, such as poultry (for example, broiler, layer, broiler breeders, turkey, duck, geese, water fowl), and swine (all age categories), a ruminant such as cattle (e.g. cows or bulls (including calves)), horses, sheep, a pet (for example dogs, cats) or fish (for example agastric fish, gastric fish, freshwater fish such as salmon, cod, trout and carp, e.g. koi carp, marine fish such as sea bass, and crustaceans such as shrimps, mussels and scallops). Preferably the feedstuff is for poultry.

The feed additive composition and/or the feedstuff comprising same may be used in any suitable form. The feed additive composition may be used in the form of solid or liquid preparations or alternatives thereof. Examples of solid preparations include powders, pastes, boluses, capsules, pellets, tablets, dusts, and granules which may be wettable, spray-dried or freeze-dried. Examples of liquid preparations include, but are not limited to, aqueous, organic or aqueous-organic solutions, suspensions and emulsions.

In some applications, the feed additive compositions may be mixed with feed or administered in the drinking water.

A feed additive composition, comprising admixing a protease as taught herein with a feed acceptable carrier, diluent or excipient, and (optionally) packaging.

The feedstuff and/or feed additive composition may be combined with at least one mineral and/or at least one vitamin. The compositions thus derived may be referred to herein as a premix.

In some embodiments, trypsin-like serine protease can be present in the feedstuff in the range of 1 ppb (parts per billion) to 10% (w/w) based on pure enzyme protein. In some embodiments, the protease is present in the feedstuff is in the range of 1-100 ppm (parts per million). A preferred dose can be 1-20 g of trypsin-like serine protease per ton of feed product or feed composition or a final dose of 1-20 ppm trypsin-like serine protease in final product.

Preferably, a trypsin-like serine protease is present in the feedstuff should be at least about 200 PU/kg or at least about 300 PU/kg feed or at least about 400 PU/kg feed or at least about 500 PU/kg feed or at least about 600 PU/kg feed, at least about 700 PU/kg feed, at least about 800 PU/kg feed, at least about 900 PU/kg feed or at least about 1000 PU/kg feed, or at least about 1500PU/kg feed, or at least about 2000PU/kg feed or at least about 2500 PU/kg feed, or at least about 3000 PU/kg feed, or at least about 3500 PU/kg feed, or at least about 4000 PU/kg feed, or at least about 4500 PU/kg feed, or at least about 5000 PU/kg feed.

In another aspect, a trypsin-like serine protease can be present in the feedstuff at less than about 60,000PU/kg feed, or at less than about 70,000PU/kg feed, or at less than about 80,000PU/kg feed, or at less than about 90,000PU/kg feed, or at less than about 100,000PU/kg feed, or at less than about 200,000PU/kg feed, or at less than about 60000PU/kg feed, or at less than about 70000 PU/kg feed.

Ranges can include, but are not limited to, any combination of the lower and upper ranges discussed above.

It will be understood that one protease unit (PU) is the amount of enzyme that liberates 2.3 micrograms of phenolic compound (expressed as tyrosine equivalents) from a casein substrate per minute at pH 10.0 at 50° C. This may be referred to as the assay for determining 1 PU.

Formulations comprising any of trypsin-like serine protease and compositions described herein may be made in any suitable way to ensure that the formulation comprises active enzymes. Such formulations may be as a liquid, a dry powder or a granule. Preferably, the feed additive composition is in a liquid form and, the liquid form may be suitable for spray-drying on a feed pellet.

Dry powder or granules may be prepared by means known to those skilled in the art, such as, high shear granulation, drum granulation, extrusion, spheronization, fluidized bed agglomeration, fluidized bed spray

Trypsin-like serine proteases and compositions described herein may be coated, for example encapsulated. In one embodiment, the coating protects the enzymes from heat and may be considered a thermo-protectant.

Feed additive composition described herein can be formulated to a dry powder or granules as described in WO2007/044968 (referred to as TPT granules) or WO1997/016076 or WO1992/012645 (each of which is incorporated herein by reference).

In one embodiment the feed additive composition may be formulated to a granule for feed compositions comprising: a core; an active agent; and at least one coating, the active agent of the granule retaining at least 50% activity, at least 60% activity, at least 70% activity, at least 80% activity after conditions selected from one or more of a) a feed pelleting process, b) a steam-heated feed pretreatment process, c) storage, d) storage as an ingredient in an unpelleted mixture, and e) storage as an ingredient in a feed base mix or a feed premix comprising at least one compound selected from trace minerals, organic acids, reducing sugars, vitamins, choline chloride, and compounds which result in an acidic or a basic feed base mix or feed premix.

With regard to the granule at least one coating may comprise a moisture hydrating material that constitutes at least 55% w/w of the granule; and/or at least one coating may comprise two coatings. The two coatings may be a moisture hydrating coating and a moisture barrier coating. In some embodiments, the moisture hydrating coating may be between 25% and 60% w/w of the granule and the moisture barrier coating may be between 2% and 15% w/w of the granule. The moisture hydrating coating may be selected from inorganic salts, sucrose, starch, and maltodextrin and the moisture barrier coating may be selected from polymers, gums, whey and starch.

The granule may be produced using a feed pelleting process and the feed pretreatment process may be conducted between 70° C. and 95° C. for up to several minutes, such as between 85° C. and 95° C.

The feed additive composition may be formulated to a granule for animal feed comprising: a core; an active agent, the active agent of the granule retaining at least 80% activity after storage and after a steam-heated pelleting process where the granule is an ingredient; a moisture barrier coating; and a moisture hydrating coating that is at least 25% w/w of the granule, the granule having a water activity of less than 0.5 prior to the steam-heated pelleting process.

The granule may have a moisture barrier coating selected from polymers and gums and the moisture hydrating material may be an inorganic salt. The moisture hydrating coating may be between 25% and 45% w/w of the granule and the moisture barrier coating may be between 2% and 10% w/w of the granule.

The granule may be produced using a steam-heated pelleting process which may be conducted between 85° C. and 95° C. for up to several minutes.

Alternatively, the composition is in a liquid formulation suitable for consumption preferably such liquid consumption contains one or more of the following: a buffer, salt, sorbitol and/or glycerol.

Also, the feed additive composition may be formulated by applying, e.g. spraying, the enzyme(s) onto a carrier substrate, such as ground wheat for example.

In one embodiment the feed additive composition may be formulated as a premix. By way of example only the premix may comprise one or more feed components, such as one or more minerals and/or one or more vitamins.

In one embodiment a direct fed microbial (“DFM”) and/or thermostable serine proteases are formulated with at least one physiologically acceptable carrier selected from at least one of maltodextrin, limestone (calcium carbonate), cyclodextrin, wheat or a wheat component, sucrose, starch, Na₂SO₄, Talc, PVA, sorbitol, benzoate, sorbate, glycerol, sucrose, propylene glycol, 1,3-propane diol, glucose, parabens, sodium chloride, citrate, acetate, phosphate, calcium, metabisulfite, formate and mixtures thereof. Non-limiting examples of compositions and methods disclosed herein include:

1. An isolated polypeptide having serine protease activity, selected from the group consisting of:

a) a polypeptide comprising an amino acid sequence with at least 91% identity with the amino acid sequence of SEQ ID NO:22;

b) a polypeptide comprising an amino acid sequence with at least 94% identity with the amino acid sequence of SEQ ID NO:23;

c) a polypeptide comprising an amino acid sequence with at least 98% identity with the amino acid sequence of SEQ ID NO:24;

d) a polypeptide comprising an amino acid sequence with at least 80% identity with the amino acid sequence of SEQ ID NO:25.

2. An isolated polypeptide having serine protease activity comprising a predicted precursor amino acid sequence selected from: SEQ ID NO:3; SEQ ID NO:6; SEQ ID NO:9; and SEQ ID NO:12.

3. An isolated polypeptide having serine protease activity comprising a protease catalytic region, selected from:

a) a protease catalytic region having an amino acid sequence of at least 96% identity with the amino acid sequence of SEQ ID NO:18;

b) an amino acid sequence with at least 98% identity with the amino acid sequence of SEQ ID NO:19;

c) an amino acid sequence of SEQ ID NO:20;

d) an amino acid sequence with at least 91% identity with the amino acid sequence of SEQ ID NO:21;

4. A recombinant construct comprising a regulatory sequence functional in a production host operably linked to a nucleotide sequence encoding at least one polypeptide having serine protease activity of embodiments 1-3.

5. The recombinant construct of embodiment 5 wherein said host is selected from the group consisting of fungi, bacteria, and algae.

6. A method for producing at least one polypeptide having serine protease comprising: (a) transforming a production host with the recombinant construct of embodiment 4; and (b) culturing the production host of step (a) under conditions whereby at least one polypeptide having serine protease activity is produced.

7. A method according to embodiment 6 wherein the serine protease is optionally recovered from the production host.

8. A serine protease-containing culture supernatant obtained by the method of any of embodiments 6 or 7.

9. A recombinant microbial production host for expressing at least one polypeptide, said recombinant microbial production host comprising the recombinant construct of embodiment 4.

10. A production host according to embodiment 9, wherein said host is selected from the group consisting of bacteria, fungi and algae.

11. Animal feed comprising at least one polypeptide of any one of embodiments 1-3, wherein said polypeptide is present in an amount from 1-20 g/ton feed.

12. The animal feed of embodiment 11, further comprising at least one direct fed microbial.

13. The animal feed of embodiment 11 or 12, further comprising at least one other enzyme.

14. A feed, feedstuff, a feed additive composition or premix comprising at least one polypeptide of any one of embodiments 1-3.

15. The feed, feedstuff, feed additive composition or premix of embodiment 14, further comprising at least one direct fed microbial.

16. The feed, feedstuff, feed additive composition or premix of embodiment 14 or 15 further comprising at least one other enzyme.

17. The feed additive composition of any one of embodiments 14-17, wherein said composition further comprises at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabisulfite, methyl paraben, and propyl paraben.

18. A granulated feed additive composition for use in animal feed comprising at least one polypeptide of any one of embodiments 1-3, wherein the granulated feed additive composition comprises particles produced by a process selected from the group consisting of high shear granulation, drum granulation, extrusion, spheronization, fluidized bed agglomeration, fluidized bed spray coating, spray drying, freeze drying, prilling, spray chilling, spinning disk atomization, coacervation, tableting, and combination thereof.

19. The granulated feed additive composition of embodiment 18, wherein the mean diameter of the particles is greater than 50 microns and less than 2000 microns.

20. The feed additive composition of any one of embodiments 14-17, wherein said composition is in a liquid form.

21. The feed additive composition of embodiment 20, wherein said composition is in a liquid form suitable for spray-drying on a feed pellet.

### EXAMPLES

Unless defined otherwise herein, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used with this disclosure.

The disclosure is further defined in the following Examples. It should be understood that the Examples, while indicating certain embodiments, is given by way of illustration only. From the above discussion and the Examples, one skilled in the art can ascertain essential characteristics of this disclosure, and without departing from the spirit and scope thereof, can make various changes and modifications to adapt to various uses and conditions.

### Example 1

### Cloning of Streptomyces sp Trypsin-Type Serine Proteases

Four bacterial strains (Streptomyces sp. C004, Streptomyces sp. C009, Streptomyces sp. C001 and Streptomyces sp. S055) were selected as potential sources of enzymes which may be useful in various industrial applications. Chromosomal DNA was isolated from the four strains and sequenced using Illumina's next generation sequencing technology. Genes encoding trypsin-like serine proteases were identified after annotation in the four aforementioned Streptomyces species; and their nucleotide or amino acid sequence identified.

The genes for all 4 proteins have an N-terminal signal peptide as predicted by SignalP software version 4.0 (Nordahl Petersen et al. (2011) Nature Methods, 8:785-786), suggesting that they are all secreted enzymes.

The nucleotide sequence of the SspCPro29 gene isolated from Streptomyces sp. C009 is set forth as SEQ ID NO:1. The predicted signal sequence of the SspCPro29 precursor protein is set forth as SEQ ID NO:2. The amino acid sequence of the SspCPro29 precursor protein is set forth as SEQ ID NO:3.

The nucleotide sequence of the SspCPro33 gene isolated from Streptomyces sp. C001 is set forth as SEQ ID NO:4: The predicted signal sequence of the SspCPro33 precursor protein is set forth as SEQ ID NO:5. The amino acid sequence of the SspCPro33 precursor protein is set forth as SEQ ID NO:6.

The nucleotide sequence of the SspCPro23 gene isolated from Streptomyces sp. C003 is set forth as SEQ ID NO:7. The predicted signal sequence of the SspCPro23 precursor protein is set forth as SEQ ID NO:8. The amino acid sequence of the SspCPro23 precursor protein is set forth as SEQ ID NO:9.

The nucleotide sequence of the SspCPro59 gene isolated from Streptomyces sp. C055 is set forth as SEQ ID NO:10. The predicted signal sequence of the SspCPro59 precursor protein is set forth as SEQ ID NO:11. The amino acid sequence of the SspCPro59 precursor protein is set forth as SEQ ID NO:12. Based on signal sequence prediction the full length amino acid sequences are predicted as follows: SspCPro29 (SEQ ID NO:22); SspCPro33 (SEQ ID NO: 23); SspCPro23 (SEQ ID NO:24); and SspCPro59 (SEQ ID NO:25).

### Example 2

### Expression of Streptomyces sp Trypsin-Type Serine Proteases

The DNA sequences encoding the propeptide-mature form (precursor protein minus signal sequence) of Streptomyces sp trypsin homologs SspCPro23, SspC29, SspC33 and SspC59 were synthesized and were each inserted into the Bacillus subtilis expression vector p2JM103BBI (Vogtentanz, Protein Expr Purif, 55:40-52, 2007) by Generay (Shanghai, China). The resulting plasmids were designated pGX384(AprE-SspCPro23), pGX390(AprE-S spCPro29), pGX394(AprE-SspCPro33) and pGX738(AprE-SspCPro59). The synthetic genes have an alternative start codon (GTG).

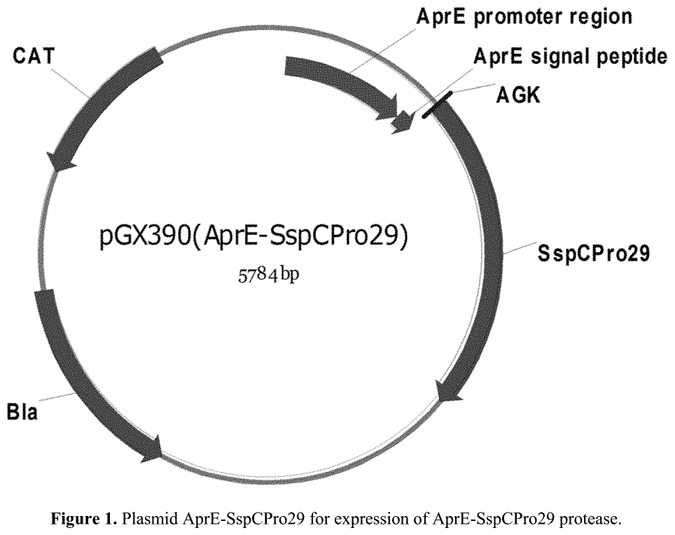

The plasmid map of pGX390(AprE-SspCPro29) is provided in FIG. 1 and the other three plasmids have similar composition with the exception of the inserted gene encoding each serine protease gene of interest (GOI). The nucleotide sequences of synthetic AprE-SspCPro23, AprE-SspCPro29, AprE-SspCPro33, AprE-SspCPro59 genes are set forth as SEQ ID NO: 13, 14, 15 and 16, respectively. Ligation of the gene encoding each GOI into the linearized expression vector resulted in the addition of three codons (encoding residues Ala-Gly-Lys) between the 3′ end of the sequence encoding the B. subtilis AprE signal and the 5′ end of the sequence encoding the propeptide-mature sequence. The AprE signal sequence (SEQ ID NO: 17) was used to direct the recombinant proteins for secretion in B. subtilis.

The expression plasmids were then transformed into suitable B. subtilis cells and the transformed cells were cultured on Luria Agar plates supplemented with 5 ppm Chloramphenicol and 1.2% skim milk (Cat #232100, Difco). Colonies forming largest clear halos were picked and used to inoculated liquid cultures. The fermentation was carried out in 250 mL shake flasks using a MOPS-based defined medium, supplemented with 5 mM CaCl₂).

For purification of SspCPro29 and SspCPro33 proteases, clarified supernatant from shake flask cultures was subjected to column chromatography using hydrophophic interaction and ion exchange resins. The resulting active protein fractions were then pooled and concentrated via the 10K Amicon Ultra devices, and stored in 40% glycerol at −20° C. until usage.

Utilizing the protein sequence annotations for the Streptomyces griseus serine protease Streptogrisin C (Uniprot accession number P52320), the various sequence regions of the SspCPro29, SspCPro 23, SspCPro 33 and SspCPro 59 were further analyzed to identify the putative amino acid residues comprising the catalytic domains of these proteases. Streptogrisin C is expressed as a 457 residue polypeptide that comprises a signal sequence (residues 1-34), a propeptide region (residues 35-202), and a mature chain (residues 203 to 457).

The mature chain is further comprised of a catalytic domain (residues 203 to 393, SEQ ID NO:39), a linker (residues 394-413) and a chitin binding region (residues 415-457). Based on this information, the catalytic domains for SspCPro29, SspCPro 23, SspCPro 33 and SspCPro 59 were predicted as: SEQ ID NO: 18, SEQ ID NO: 19, SEQ ID NO: 20, and SEQ ID NO: 21, respectively.

### Example 3

### Proteolytic Activity of Streptomyces sp Trypsin-Type Serine Proteases

The proteolytic activities of purified SspCPro29 and SspCPro33 were measured in 50 mM HEPES buffer (pH 8), using Suc-Ala-Ala-Pro-Phe-pNA (AAPF-pNA, Cat # L-1400.0250, BACHEM) as a substrate. A sample of the commercial product RONOZYME® ProAct protease (DSM) was used as a reference. Prior to the reaction, the enzymes were diluted with Purified water (Millipore) to specific concentrations. The AAPF-pNA was dissolved in Dimethyl sulfoxide (DMSO, Cat # STBD2470V, Sigma) to a final concentration of 10 mM.

To initiate the reaction, firstly 5 μL of AAPF-pNA was mixed with 85 μL of HEPES buffer in a non-binding 96-well microtiter plate (96-MTP) (Corning Life Sciences, #3641) and incubated at 40° C. for 5 min at 600 rpm in a Thermomixer (Eppendorf), then 10 μL of the diluted enzyme (or Purified H2O2O alone as the blank control) was added. After 10 min incubation in a Thermomixer at 40° C. and 600 rpm, the reaction plate was directly read at 410 nm using a SpectraMax 190. Net A410 was calculated by subtracting the A410 of the blank control from that of enzyme, and then plotted against different protein concentrations (from 0.02 ppm to 0.3125 ppm). Each value was the mean of triplicate assays.

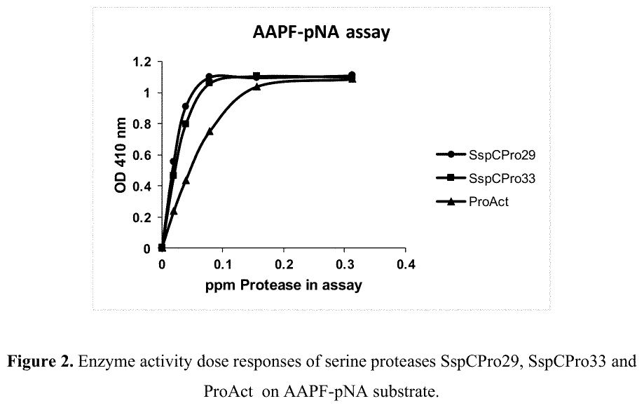

The proteolytic activity on AAPF-pNA substrate is shown on FIG. 2 as Net A41o. The proteolytic activities of SspCPro23 and SsCPro59 were measured using clarified supernatant from shake flask cultures. The clarified culture supernatant of B. subtilis cells transformed with p2JM103BBI (lacking a protease gene) was used as the vector control. Prior to the reaction, the supernatants were diluted 200 fold with purified water. The assay procedure was carried out as described above, and the Net A410 was calculated by subtracting the A410 of the vector control from that of enzyme sample. Each value was the mean of triplicate assays. The proteolytic activity is shown as Net A410 on Table 1, indicating that SspCPro23 and SspCPro59 are active proteases.

TABLE 1
Enzyme activity of SspCPro59 and
SspCPro23 on pNA-AAPF substrate

| Protein ID | Net absorbance 410 nm |
|---|---|
| SspCPro23 | 0.24 |
| SspCPro59 | 0.27 |

### Example 4

### pH Profile of Streptomyces sp Trypsin-Type Serine Proteases

With AAPF-pNA as the substrate, the pH profile of trypsin homologs was studied in 25 mM glycine/sodium acetate/HEPES buffer with different pH values (ranging from pH 3 to 10). Prior to the assay, 85 μL of 25 mM glycine/sodium acetate/HEPES buffer with a specific pH value was first mixed with 5 μL of 10 mM AAPF-pNA in a 96-MTP, and then 10 μL of purified water. Diluted enzyme (0.2 ppm for purified SspCPro29 and SspCPro33, or clarified supernatant of SspCPro23 and SspCPro59 diluted 200 fold) was then added to initiate the reaction. Water, or supernatant from vector control (200 fold diluted) were used as the blank control for purified or unpurified enzymes, respectively. The reaction was performed and analyzed as described in Example 3. Enzyme activity at each pH was reported as relative activity where the activity at the optimal pH was set to be 100%. The pH values tested for purified enzymes (SspCPro29 and SspCPro33) were 3, 4, 5, 6, 7, 8, 9 and 10; and for the unpurified (SspCPro23 and SspCPro59) were 3, 5.5, 8, 9, 10. Each value was the mean of triplicate assays.

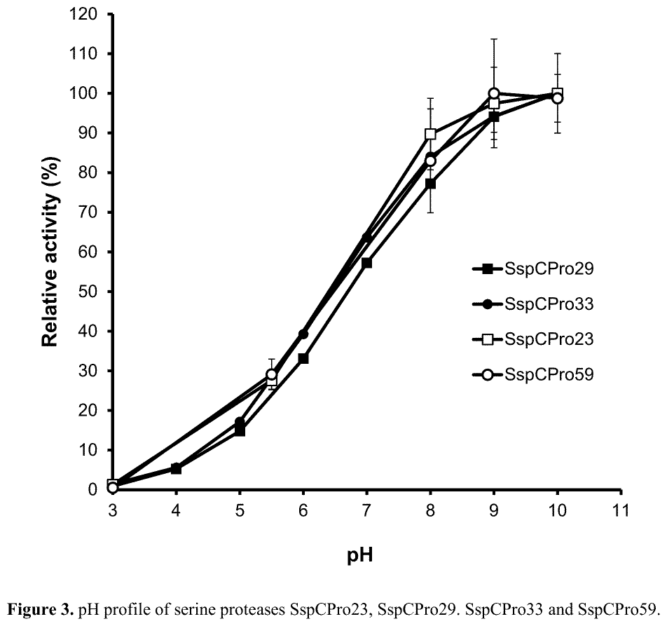

As shown in FIG. 3, all the trypsin homologs were alkaline proteases.

### Example 5

### Temperature Profile of Streptomyces sp Trypsin-Type Serine Proteases

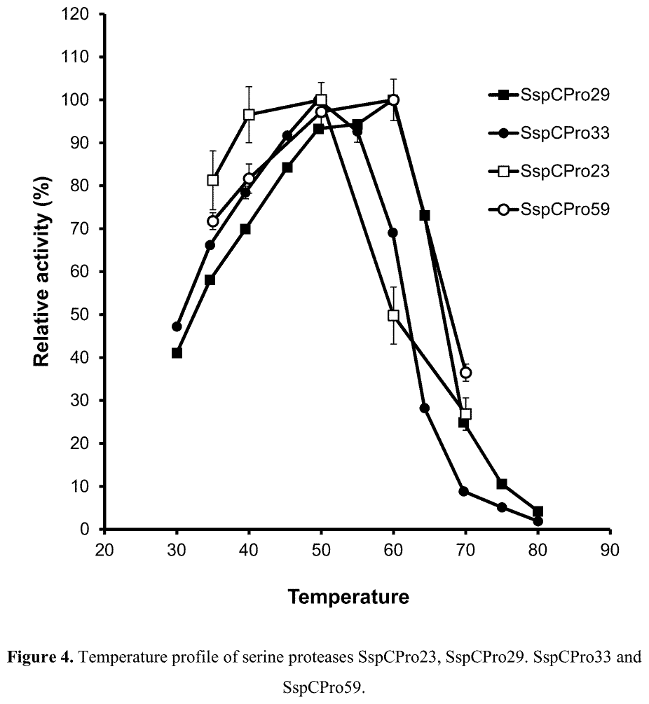

The temperature profile of trypsin homologs was analyzed in 50 mM HEPES buffer (pH 8) using the AAPF-pNA assay. Prior to the reaction, 85 μL of 50 mM pH 8.0 HEPES buffer and 5 μL of 10 mM AAPF-pNA were added in a 200 μL PCR tube, which was then subsequently incubated in a Peltier Thermal Cycler (BioRad) at desired temperatures (between 30-80° C.) for 5 min. After the incubation, 10 μL of enzyme sample (0.2 ppm for purified SspCPro29 and SspCPro33, and clarified supernatant (200 fold diluted) for SspCPro23 and SspCPro59) was added to the substrate to initiate the reaction. Water alone or supernatant from vector control (200 fold diluted) was added as the blank control for purified or unpurified enzymes, respectively. Following 10 min incubation in the Peltier Thermal Cycler at different temperatures, 80 μL of the reaction mixture was transferred to a new 96-MTP and the absorbance was read at 410 nm. The activity was reported as relative activity where the activity at the optimal temperature was set to be 100%. The tested temperatures for purified enzymes (SspCPro29 and SspCPro33) were 30, 35, 40, 45, 50, 55, 60, 65, 70, 75 and 80° C.; and for unpurified proteins SspCPro23 and SspCPro59 were tested at 35, 40, 50, 60 and 70° C. Each value reported is the mean of triplicate assays. Results are shown in FIG. 4.

### Example 6

### Corn Soy Meal Hydrolysis by Streptomyces sp Trypsin-Type Serine Proteases

The extent of corn soy meal (a mixture having 40% soy meal and 60% corn flour) hydrolysis by the trypsin homologs was evaluated using the OPA (o-phthalaldehyde) or the BCA (bicinchoninic acid) detection assays described below, to measure the amount of newly produced N-terminal amine groups or soluble peptides, respectively, released into the supernatant after the enzymatic reactions. To conduct the assays, 140 μL of the corn soy meal substrate (10% (w/w) corn soy meal slurry suspended in MES pH 6 buffer) was mixed with 20 μL of a diluted purified enzyme sample (SspCPro29 and SspCPro33) or with 60 μL of the clarified supernatant (SspCPro23 and SspCPro59)-in a 96-MTP. After incubation for 2 hrs at 40° C. in an incubator, the plates were centrifuged at 3700 rpm for 15 min at 4° C. The resulting supernatant was diluted 10 times in water to prepare for subsequent reaction product detection using the OPA and BCA assays. For purified proteases, a sample of RONOZYME® ProAct protease (DSM) was included as the commercial benchmark protease and water was used as the (no enzyme) blank control. For the unpurified, the supernatant from vector control was applied as the blank control.

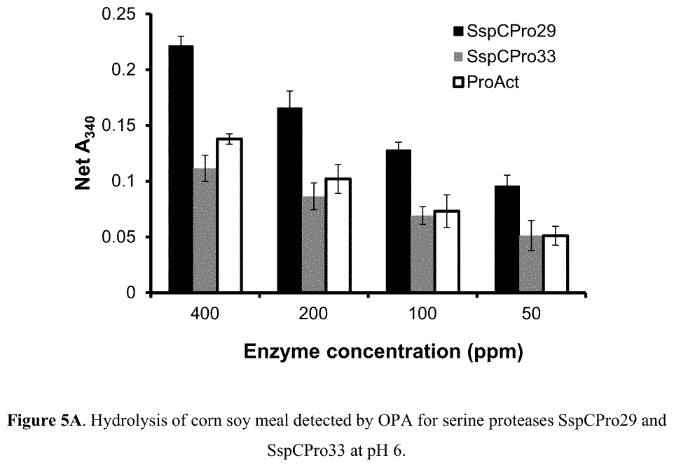

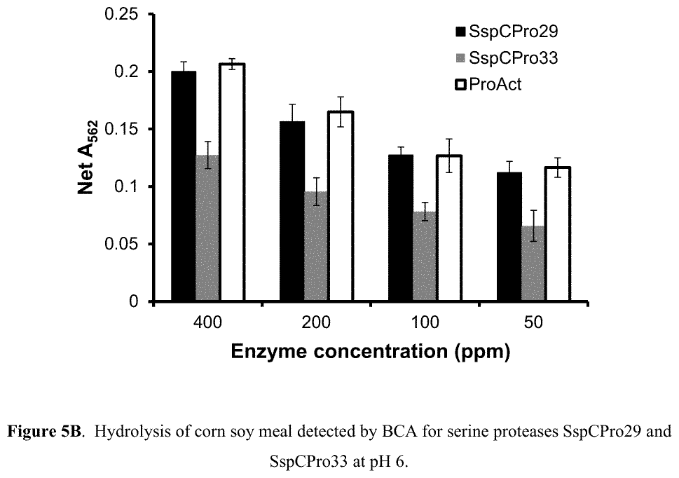

The OPA reagent was prepared by mixing 30 mL of 2% tri-sodium phosphate buffer (pH 11), 800 μL of 4% OPA (Cat # P1378, Sigma, dissolved in 96% ethanol), 1 mL of 3.52% dithiothreitol (Cat # D0632, Sigma), and 8.2 mL of H₂O. The reaction was initiated by adding 10 μL of the 10× diluted protease reaction to 175 μL OPA reagent in a 96-MTP (Cat #3635, Corning Life Sciences). After 2 min incubation at 20° C., the absorbance of the resulting solution was measured at 340 nm (A₃₄₀) using a spectrophotometer. The net A₃₄₀ was calculated by subtracting the A₃₄₀ of the blank (water for SspCPro29 and SspCPro33; supernatant from vector control for SspCPro23 and SspCPro59) control from that of each protease reaction, to measure the extent of corn soy meal hydrolysis achieved by each protease sample. The results are shown on FIG. 5A and Table 2.

The BCA reaction was conducted by mixing 10 μL diluted supernatant with 200 μL BCA reagent following manufacturer guidelines The incubation was conducted in a thermomixer at 37° C. for 30 min and the product of the reactions were measured in a spectrophotometer as an endpoint absorbance reading at 562 nm. The net A562 was calculated by subtracting the A562 of the blank (water for SspCPro29 and SspCPro33; supernatant from vector control for SspCPro23 and SspCPro59) control from that of each protease reaction, to measure the extent of corn soy meal hydrolysis achieved by each protease sample. The results are shown on FIG. 5B and Table 2.

TABLE 2
OPA and BCA corn soy meal hydrolysis by
SspCPro23 and SspCPro59 proteases at pH 6

| | OPA (Net₃₄₀) | BCA (Net₅₆₂) |
|---|---|---|
| SspCPro23 | 0.04 | 0.04 |
| SspCPro59 | 0.06 | 0.06 |

### Example 7

### Pepsin Stability of Streptomyces sp Trypsin-Type Serine Proteases

Pepsin stability of trypsin homologs was analyzed by incubating them with pepsin (Sigma, Cat. No. P7000) in 50 mM sodium acetate buffer (pH 3.0) and using AAPF-pNA as the substrate for remaining activity measurement. Trypsin homologs and pepsin were first mixed in ratios (w/w) of 1:0, 1:25, 1:250 or 1:2500, where the trypsin homologs were dosed at 20 ppm; and the resulting mixture was subsequently incubated at 37° C. for 30 min. Meanwhile, 20 ppm aliquots of each trypsin homolog were kept on ice as untreated controls. For remaining activity measurement, 5 μL of 10 mM AAPF-pNA was mixed with 85 μL of HEPES buffer (50 mM, pH 8.0) in a 96-MTP, then 10 μL of the purified water diluted mixture (0.2 ppm or H₂O alone as the blank control) was added. The reaction was performed and analyzed as described in Example 3.

Table 3 shows the residual enzyme activity following pepsin treatment, where the activity of the untreated samples kept on ice was set to 100%.

TABLE 3
Pepsin stability of serine proteases SspCPro29 and SspCPro33

| | | | trypsin:pepsin | | |
|---|---|---|---|---|---|
| | | trypsin | (ratios) | | |
| Sample | untreated | only | 1:25 | 1:250 | 1:2500 |
| SspCPro29 | 100 | 82 | 87 | 97 | 104 |
| SspCPro33 | 100 | 97 | 95 | 98 | 106 |
| ProAct | 100 | 101 | 102 | 100 | 94 |

### Example 8

### Stability of SspCPro29 Protease Under Feed Pelleting Conditions

The pelleting conditions were as follows: 62.5 g of a concentrated solution of SspCPro29 protease consisting of 38.37 g protein was diluted in tap water to 600 mL, and mixed with 120 kg of corn soy feed (60% Corn, 31.5% Soybean meal, 4.0% Soy oil, 0.4% Salt, 0.2% DL-Methionine, 1.16% Limestone, 1.46% calcium phosphate, and 1.25% vitamin and mineral mixture, by weight. This mixture was pelleted at 90° C., or 95° C. for 30 seconds. A similarly prepared mixture of enzyme sample and corn soy feed (mash feed) that did not undergo pelleting serves as control. The extraction conditions were as follows: 1 g pelleted feed or mash feed was grinded, mixed with 10 mL buffer (100 mM Tris buffer with 1% SDS pH 10), in 10 mL beaker with a magnetic stir bar at room temperature (22° C.) for 10 min, then centrifuged at 4000 rpm using a bench top centrifuge for 10 min. The supernatant was filtered through a glass fiber filter. The filtrate was directly used in the enzyme activity assay. The enzyme activity assay conditions were as follows: 0.18 mL 0.1M Tricine buffer (pH 9.75 with 1% SDS), 1 μL enzyme feed extract, and 20 μL AAPF-pNA substrate (10 mg/ml in DMSO) were mixed at 900 rpm for 1 min. Samples were incubated at 30° C. for 120 min with constant shaking.

The extent of the reaction was determined by measuring absorbance at 410 nm in a spectrophotometer. Results are shown on Table 4.

TABLE 4
Pelleting stability of SspCPro29 measured as recovery
of enzyme activity from pellets versus untreated mash

| Sample | % recovery | cv |
|---|---|---|
| no pelleting | 100 | 3.6 |
| 90° C. pelleting | 78.7 | 3.6 |
| 95° C. pelleting | 62.0 | 4.1 |

### Example 9

### Hydrolysis and Solubilization of Protein in Corn Soy Feed with SspCPro29 Protease and SspCPro33 Protease

The reaction contained in 96 well MTP 1404, 10% (w/w) corn soy feed slurry (Yu S, Cowieson A, Gilbert C, Plumstead P, Dalsgaard S., Interactions of phytate and myo-inositol phosphate esters (IP1-5) including IP5 isomers with dietary protein and iron and inhibition of pepsin. J. Anim. Sci. 2012, 90:1824-1832) with pH adjusted to pH 3.0, 204, the protease in 50 mM Na-acetate pH3.0 giving a final concentration to the corn soy feed at 0, 250, 500, 750, 1000 and 1250 ppm, and 10 μL pepsin (Sigma P7000 dissolved in water at 1.69 mg/ml). The plate was incubated at 40° C. for 45 min in an iEMS Incubator/Shaker (Thermo Scientific) at 1150 rpm. At the end of the incubation porcine pancreatin (Sigma P7545, 0.4636 mg/mL in 1M sodium bicarbonate) 34 μL was added and the plate was further incubated at 40° C. for 60 mi in the iEMS shaking at 1150 rpm. After the incubation, the plate was centrifuged at 5° C., 4000 rpm for 15 min. The supernatant 10 μL supernatant was transferred to new 96 well MTP containing 904, water (10× dilution). The 10 time diluted supernatant was determined for OPA (protein hydrolysis) and BCA (protein solubilization) values at 340 nm and 562 nm, respectively.

Protein hydrolysis using o-phthaldialdehyde (OPA) reagent was done basically as described before with minor modifications (P. M. NIELSEN, D. PETERSEN, and C. DAMBMANN, Improved method for determining food protein degree of hydrolysis, J. Food Sci. 66:642-646, 2001). The OPA reagent was prepared freshly by mixing 30 mL tri-sodium phosphate (Na3PO4.12H₂O, 2% w/v in water with pH adjusted pH11), 0.8 mL OPA (0.4 g o-phthaldialdehyde 97% (OPA) in 10 mL 96% ethanol, saved at −20° C.), 1 ml DTT solution (0.352 g DL-dithiothreitol (DTT) 99% in 10 mL water and water to a final volume of 40 mL. The reagent was kept in the dark and used right after the preparation. The 10x diluted supernatant 204, was mixed with 1754, of the OPA reagent for 5 seconds and read at 340 nm exactly after 2 min.

Protein solubilization was measured by using the Pierce BCA Protein Assay Kit (Cat no. 23225 from Thermo Fisher Scientific). The supernatant 204, was mixed with 2004, of the BCA reagent (prepared before use by mixing 50 mL BCA reagent A with 1 mL BCA regent B according to the manufacturer's instruction). The mixture was incubated at 37° C. for 30 min before absorbance at 562 nm was measured.

Tables 5.1 and 5.2 show that protein hydrolysis and protein solubilization in the corn soy feed increased with the increase of the SspCPro29 and SspCPro33 (respectively) protease dose from 0 to 1250 ppm in the presence of both pepsin and pancreatin. The respective correlation coefficient (R²) for hydrolysis and solubilization was 0.90 and 0.97.

TABLE 5.1
Hydrolysis and protein solubilization of
corn soy feed with SspCPro29 protease

| | | | | | | | Correlation |
|---|---|---|---|---|---|---|---|
| | Enzyme concentration(ppm) | | | | | | coefficient |
| | 0 | 250 | 500 | 750 | 1000 | 1250 | (R²) |
| Protein | 0.529 | 0.689 | 0.761 | 0.806 | 0.835 | 0.884 | 0.900 |
| hydrolysis | | | | | | | |
| (OD340 OPA | | | | | | | |
| assay) | | | | | | | |
| Protein | 0.818 | 0.856 | 0.897 | 0.911 | 0.936 | 0.992 | 0.970 |
| solubilization | | | | | | | |
| (OD 562 BCA | | | | | | | |
| assay) | | | | | | | |

TABLE 5.2
Hydrolysis and protein solubilization of
corn soy feed with SspCPro33 protease

| | | | | | | | Correlation |
|---|---|---|---|---|---|---|---|
| | Enzyme concentration(ppm) | | | | | | coefficient |
| | 0 | 250 | 500 | 750 | 1000 | 1250 | (R²) |
| Protein | 0.513 | 0.634 | 0.682 | 0.698 | 0.717 | 0.753 | 0.854 |
| hydrolysis | | | | | | | |
| (OD340 OPA | | | | | | | |
| assay) | | | | | | | |
| Protein | 0.806 | 0.807 | 0.844 | 0.839 | 0.844 | 0.895 | 0.818 |
| solubilization | | | | | | | |
| (OD 562 BCA | | | | | | | |
| assay) | | | | | | | |

### Example 10

### Cleaning Performance of SspCPro29 and SspCPro33 in ADW

The cleaning performance of SspCPro29 and SspCPro33 proteases was tested using PA-S-38 (egg yolk, with pigment, aged by heating) microswatches (CFT-Vlaardingen, The Netherlands) at pH 10.3 using a model automatic dishwashing (ADW) detergent. To prepare rinsed PAS38 swatches, 180 μL 10 mM CAPS buffer (pH 11) was added to 96-MTPs containing PAS38 swatches. The plates were sealed and incubated in an iEMs incubator for 30 min at 60° C., 1100 rpm. After incubation the buffer was removed and the swatches were rinsed with purified H₂O. The plates were air dried prior to use in the performance assay.

Purified protease samples were diluted to 200 ppm in 10 mM NaCl containing 0.1 mM CaCl₂) and 0.005% TWEEN® 80. The tests were performed in 3 g/L GSM-B detergent. The composition of GSM-B detergent (in weight percent) is as follows: 30% sodium citrate dehydrate, 25% sodium disilicate (Protil A, Cognis), 12% maleic acid/acrylic acid copolymer sodium Salt (SOKALAN® CP5 BASF), 5% sodium perborate monohydrate, 2% TAED, 2% linear fatty alcohol ethoxylate, and sodium carbonate anhydrous added to 100%. A190 μL aliquot of the GSM-B detergent was added to a 96-MTP containing 1 rinsed PAS38 microswatch per well, and the reaction was initiated by the addition of 10 μL of diluted enzymes (or the dilution solution alone as the blank control). The 96-MTP was sealed and placed an incubator/shaker for 30 min at 40° C. and 1150 rpm. After incubation, 100 μL of wash liquid from each well was transferred to a new 96-MTP, and its absorbance was measured at 405 nm using a spectrophotometer. The protease activity on the PAS38 model stain is reported as Net A₄₀₅, by subtracting the A₄₀₅ of the blank control from that of enzyme treated sample.

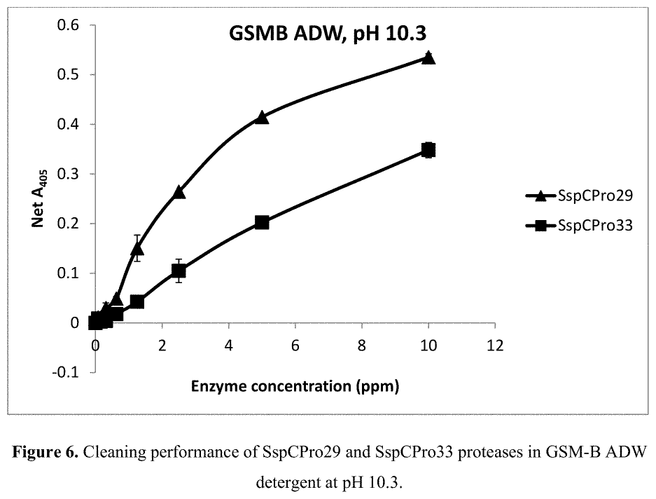

Dose responses in the PAS38 microswatches using GSM-B detergent at pH 10.3 for SspCPro29 and SspCPro33 are shown in FIG. 6.

### Example 11

### Cleaning Performance of SspCPro29 and SspCPro33 in Laundry Conditions

The cleaning performance of SspCPro29 and SspCPro33 proteases in liquid and powder laundry detergent was tested using EMPA-116 (cotton soiled with blood/milk/ink) microswatches (obtained from CFT Vlaardingen, The Netherlands) at pH 8.0 or pH 10.0. Prior to the tests, commercial liquid detergent (Tide Clean Breeze®, Proctor & Gamble, USA) was incubated at 95° C. for 1 hour to inactivate the enzymes present in the detergent. The heat treated detergent was further diluted with 5 mM HEPES (pH 8.0) to a final concentration of 0.788 g/L. The water hardness of this buffered liquid detergent was adjusted to 100 ppm Ca²⁺: Mg²⁺ (3:1 ratio). For buffered powder detergent preparation, the commercial detergent (Tide®, Proctor & Gamble, China) was dissolved to 2 g/L in water with 100 ppm water hardness at and heated in a microwave to mere boiling to inactivate enzymes. Proteolytic assays were subsequently performed to confirm the inactivation of proteases in the commercial detergents.

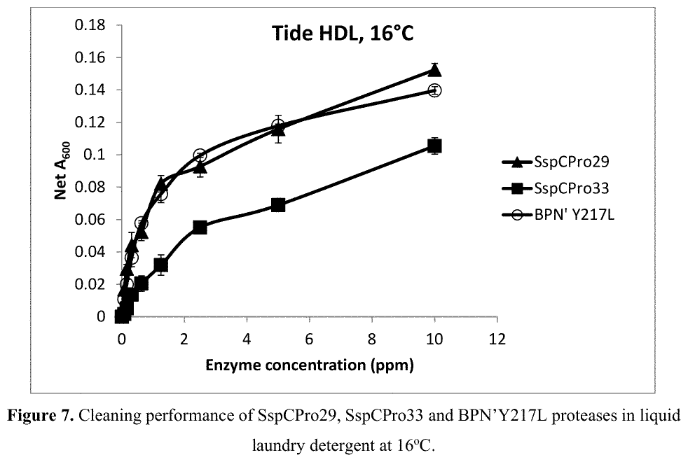

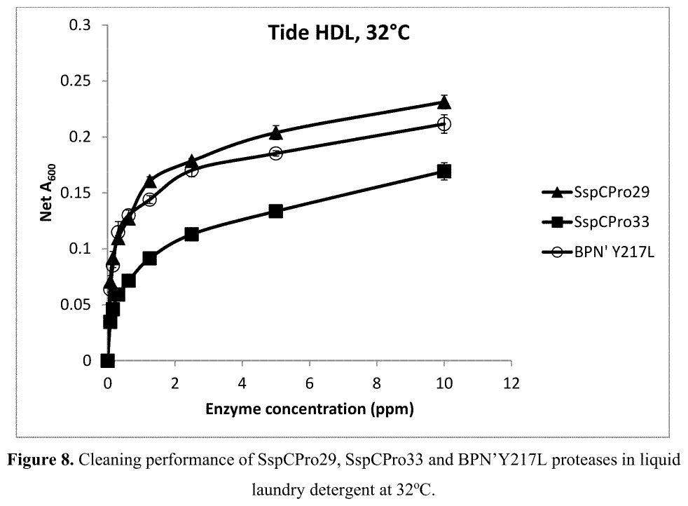

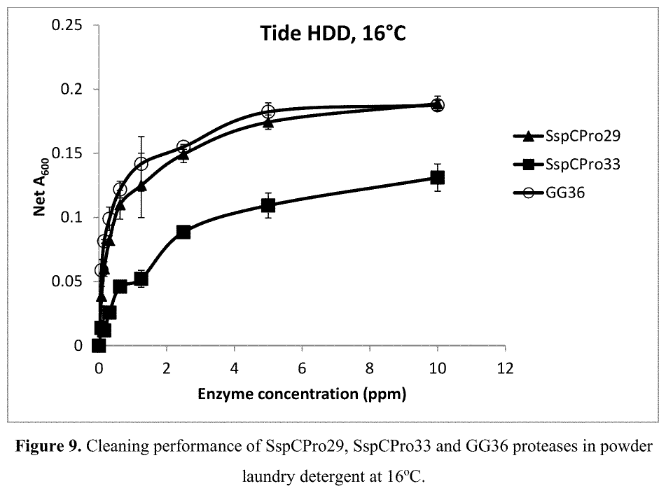

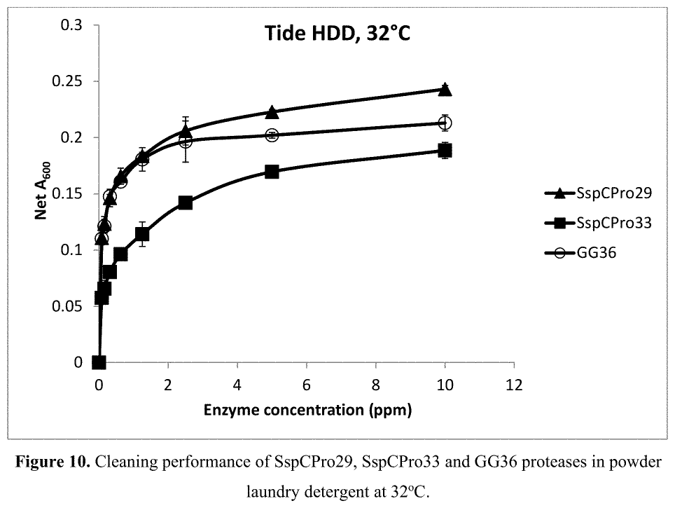

Prior to the tests, the EMPA-116 microswatches were rinsed with water and air dried. To initiate the reactions, 190 μL of buffered detergent was added to 96-MTPs wells containing the rinsed EMPA-116 microswatches, followed by the addition of 10 μL of diluted enzyme (or H₂O as blank control). The 96-MTPs were sealed and incubated for 20 min in iEMs at 32° C. and in Thermomixer at 16° C., respectively. After incubation, 100 μL of wash liquid from each well was transferred to new 96-MTP, and the absorbance was measured at 600 nm using a spectrophotometer. The Net A₆₀₀ was calculated by subtracting the A₆₀₀ of the blank control from that of the enzyme treated samples. Dose response curves for SspCPro29 and SspCPro33 on EMPA-116 microswatches in liquid and powder laundry detergent at were performed 16° C. and 32° C. The BPN′ Y217L protease (SEQ ID NO:40) was used as reference for HDL evaluation, and the GG36 protease (SEQ ID NO:41) was used as reference for HDD evaluation. The cleaning performance results are shown in FIG. 7, FIG. 8, using the HDL detergent at 16 and 32° C., and in FIG. 9 and FIG. 10 using the HDD detergent at 16 and 32° C.

### Example 12

### Protein Sequence Analyses of Predicted Full Length Streptomyces sp Trypsin-Type Serine Proteases

Related proteins were identified by a BLAST search (Altschul et al., Nucleic Acids Res, 25:3389-402, 1997) using the predicted full length amino acid sequences for SspCPro29 (SEQ ID NO:22); SspCPro33 (SEQ ID NO: 23); SspCPro23 (SEQ ID NO:24); and SspCPro59 (SEQ ID NO:25) against Public and Genome Quest Patent databases with search parameters set to default values and a subset are shown on Tables 6A and 6B (SspCPro29); Tables 7A and 7B (SspCPro33); Tables 8A and 8B (SspCPro23); and Tables 9A and 9B (SspCPro59) respectively. Percent identity (PID) for both search sets is defined as the number of identical residues divided by the number of aligned residues in the pairwise alignment. Value labeled “Sequence length” on tables corresponds to the length (in amino acids) for the proteins referenced with the listed Accession numbers, while “Aligned length” refers to sequence used for alignment and PID calculation.

TABLE 6A
List of sequences with percent identity to SspCPro29 full length
protein identified from the NCBI non-redundant protein database

| | | | Sequence | Alignment |
|---|---|---|---|---|
| Accession # | PID | Organism | Length | Length |
| WP_064069271 | 90.8 | Streptomyces albulus | 453 | 426 |
| WP_030548298 | 80.7 | Streptomyces albus | 459 | 424 |
| WP_005320871 | 79.3 | Streptomyces | 453 | 430 |
| | | pristinaespiralis | | |
| WP_026277977 | 79.0 | Streptomyces sp. | 458 | 428 |
| | | CNT372 | | |
| WP_019886521 | 75.7 | Streptomyces | 463 | 432 |
| | | purpureus | | |
| WP_029386953 | 75.3 | Streptomyces | 394 | 393 |
| | | leeuwenhoekii | | |
| WP_030027622 | 75.1 | Streptomyces | 348 | 353 |
| | | flavotricini | | |
| WP_030212164 | 74.8 | Streptomyces | 454 | 421 |
| | | bikiniensis | | |
| WP_055639793 | 74.8 | Streptomyces | 451 | 428 |
| | | venezuelae | | |

TABLE 6B
List of sequences with percent identity to SspCPro29 full
length protein identified from Genome Quest database

| | | | | Align. |
|---|---|---|---|---|
| GQ Identifier | PID | Organism | Length | length |
| US8076468-0024 | 79.5 | Streptomyces griseus | 255 | 253 |
| WO2015048332-44022 | 79.3 | Streptomyces | 453 | 463 |
| | | pristinaespiralis | | |
| | | ATCC 25486 | | |
| EP2205730-0009 | 77.7 | Streptomyces sp. | 256 | 255 |

TABLE 7A
List of sequences with percent identity to SspCPro33 full length
protein identified from the NCBI non-redundant protein database

| | | | Sequence | Alignment |
|---|---|---|---|---|
| Accession # | PID | Organism | Length | Length |
| WP_043225562 | 93.7 | Streptomyces sp. NRRL F-5193 | 456 | 426 |
| WP_031004112 | 92.7 | Streptomyces sp. NRRL F-5727 | 454 | 426 |
| WP_030498660 | 91.8 | Microtetraspora glauca | 455 | 426 |
| WP_030749137 | 87.3 | Streptomyces griseus | 456 | 426 |
| WP_030212164 | 86.2 | Streptomyces bikiniensis | 454 | 419 |
| WP_062759972 | 85.2 | Streptomyces sp. WAC04657 | 454 | 419 |
| WP_030027622 | 84.5 | Streptomyces flavotricini | 348 | 349 |
| WP_053644256 | 83.8 | Streptomyces sp. NRRL F-6492 | 455 | 419 |
| WP_030313004 | 83.2 | Streptomyces flavochromogenes | 456 | 422 |
| WP_015038204 | 82.9 | Streptomyces venezuelae ATCC | 456 | 422 |
| | | 10712 | | |
| WP_055639793 | 82.8 | Streptomyces venezuelae | 451 | 425 |
| WP_030016658 | 82.7 | Streptomyces lavendulae | 369 | 369 |
| WP_055599201 | 82.6 | Streptomyces aureus | 456 | 420 |
| WP_053685358 | 82.6 | Streptomyces sp. XY593 | 451 | 419 |
| WP_024756173 | 82.4 | Streptomyces exfoliatus | 451 | 425 |
| WP_030658602 | 82.3 | Streptomyces sp. H036 | 451 | 419 |
| WP_053627230 | 82.1 | Streptomyces sp. XY511 | 451 | 419 |
| WP_030965679 | 81.9 | Streptomyces sp. NRRL S-378 | 449 | 419 |
| WP_030208917 | 81.8 | Streptomyces griseoluteus | 456 | 424 |
| WP_017236541 | 81.8 | Streptomyces sp. SS | 456 | 424 |
| WP_056557852 | 81.8 | Streptomyces sp. Root66D1 | 454 | 418 |
| WP_030545445 | 81.6 | Streptomyces exfoliatus | 456 | 424 |
| WP_033200913 | 81.6 | Streptomyces viridochromogenes | 456 | 424 |
| WP_046779091 | 81.5 | Streptomyces yangpuensis | 451 | 426 |
| WP_033218333 | 81.4 | Streptomyces virginiae | 449 | 420 |
| WP_031153386 | 81.4 | Streptomyces erythrochromogenes | 448 | 419 |
| WP_030850543 | 81.2 | Streptomyces | 450 | 426 |
| WP_053171320 | 81.1 | Streptomyces virginiae | 449 | 419 |
| WP_030896075 | 81.0 | Streptomyces virginiae | 451 | 426 |
| BAU88265 | 81.0 | Streptomyces laurentii | 445 | 426 |
| WP_053705101 | 81.0 | Streptomyces sp. WM6368 | 449 | 420 |
| WP_030385747 | 81.0 | Streptomyces sp. NRRL S-241 | 449 | 420 |
| WP_045323790 | 80.9 | Streptomyces sp. NRRL F-4428 | 449 | 425 |
| WP_053634074 | 80.8 | Streptomyces sp. MMG1064 | 451 | 426 |
| WP_053679192 | 80.8 | Streptomyces sp. XY66 | 451 | 426 |
| WP_030829885 | 80.8 | Streptomyces sp. NRRL S-104 | 451 | 426 |
| WP_030712260 | 80.7 | Streptomyces sp. NRRL S-237 | 449 | 420 |
| WP_037919299 | 80.6 | Streptomyces sp. PCS3-D2 | 454 | 428 |
| WP_053632580 | 80.5 | Streptomyces sp. H021 | 451 | 426 |
| WP_052876505 | 80.1 | Streptomyces sp. NRRL F-4335 | 451 | 422 |
| WP_030774478 | 80.0 | Streptomyces sp. NRRL F-2664 | 450 | 426 |
| WP_031144485 | 80.0 | Streptomyces xanthophaeus | 447 | 419 |
| WP_007266194 | 77.8 | Streptomyces sp. C | 455 | 427 |
| WP_005320871 | 76.8 | Streptomyces pristinaespiralis | 453 | 431 |
| WP_030548298 | 75.9 | Streptomyces albus | 459 | 428 |
| WP_026277977 | 75.1 | Streptomyces sp. CNT372 | 458 | 430 |
| WP_019886521 | 74.7 | Streptomyces purpureus | 463 | 430 |
| WP_029386953 | 74.6 | Streptomyces leeuwenhoekii | 394 | 393 |

TABLE 7B
List of sequences with percent identity to SspCPro33 full
length protein identified from Genome Quest database

| | | | | Align. |
|---|---|---|---|---|
| GQ Identifier | PID | Organism | Length | length |
| WO2015048332-44360 | 82.9 | Streptomyces | 456 | 422 |
| | | venezuelae | | |
| WO2015048332-44127 | 77.8 | Streptomyces sp. C | 455 | 427 |
| WO2015048332-44022 | 76.8 | Streptomyces | 453 | 431 |
| | | pristinaespiralis | | |
| | | ATCC 25486 | | |
| US8076468-0024 | 76.0 | Streptomyces griseus | 255 | 254 |
| US8076468-0009 | 75.0 | Streptomyces sp.; | 256 | 256 |
| | | Strain 1AG3 | | |

TABLE 8A
List of sequences with percent identity to SspCPro23 full length
protein identified from the NCBI non-redundant protein database

| | | | Sequence | Alignment |
|---|---|---|---|---|
| Accession # | PID | Organism | Length | Length |
| WP_024756173 | 97.1 | Streptomyces exfoliatus | 451 | 421 |
| WP_055639793 | 94.8 | Streptomyces venezuelae | 451 | 421 |
| WP_030313004 | 89.4 | Streptomyces flavochromogenes | 456 | 425 |
| WP_033200913 | 89.3 | Streptomyces viridochromogenes | 456 | 422 |
| WP_017236541 | 89.1 | Streptomyces sp. SS | 456 | 422 |
| WP_055599201 | 89.0 | Streptomyces aureus | 456 | 418 |
| WP_030545445 | 88.5 | Streptomyces exfoliatus | 456 | 419 |
| WP_030208917 | 88.5 | Streptomyces griseoluteus | 456 | 419 |
| WP_015038204 | 88.3 | Streptomyces venezuelae ATCC | 456 | 419 |
| | | 10712 | | |
| WP_056557852 | 87.7 | Streptomyces sp. Root66D1 | 454 | 423 |
| WP_053644256 | 85.7 | Streptomyces sp. NRRL F-6492 | 455 | 419 |
| WP_062759972 | 85.4 | Streptomyces sp. WAC04657 | 454 | 419 |
| WP_030212164 | 85.4 | Streptomyces bikiniensis | 454 | 419 |
| BAU88265 | 85.0 | Streptomyces laurentii | 445 | 419 |
| WP_030749137 | 84.6 | Streptomyces griseus | 456 | 422 |
| WP_043225562 | 84.6 | Streptomyces sp. NRRL F-5193 | 456 | 422 |
| WP_031004112 | 84.5 | Streptomyces sp. NRRL F-5727 | 454 | 420 |
| WP_030498660 | 83.6 | Microtetraspora glauca | 455 | 421 |
| WP_030027622 | 83.3 | Streptomyces flavotricini | 348 | 347 |
| WP_053685358 | 81.8 | Streptomyces sp. XY593 | 451 | 417 |
| WP_030658602 | 81.5 | Streptomyces sp. H036 | 451 | 417 |
| WP_037919299 | 81.5 | Streptomyces sp. PCS3-D2 | 454 | 416 |
| WP_053627230 | 81.3 | Streptomyces sp. XY511 | 451 | 417 |
| WP_030016658 | 81.0 | Streptomyces lavendulae | 369 | 368 |
| WP_045323790 | 80.9 | Streptomyces sp. NRRL F-4428 | 449 | 419 |
| WP_030965679 | 80.7 | Streptomyces sp. NRRL S-378 | 449 | 420 |
| WP_007266194 | 80.4 | Streptomyces sp. C | 455 | 424 |
| WP_030896075 | 80.3 | Streptomyces virginiae | 451 | 421 |
| WP_053634074 | 80.3 | Streptomyces sp. MMG1064 | 451 | 421 |
| WP_053679192 | 80.3 | Streptomyces sp. XY66 | 451 | 421 |
| WP_053705101 | 80.2 | Streptomyces sp. WM6368 | 449 | 420 |
| WP_031153386 | 80.2 | Streptomyces erythrochromogenes | 448 | 419 |
| WP_031144485 | 80.1 | Streptomyces xanthophaeus | 447 | 418 |
| WP_046779091 | 80.0 | Streptomyces yangpuensis | 451 | 421 |
| WP_030829885 | 80.0 | Streptomyces sp. NRRL S-104 | 451 | 421 |
| WP_053632580 | 80.0 | Streptomyces sp. H021 | 451 | 421 |
| WP_030385747 | 80.0 | Streptomyces sp. NRRL S-241 | 449 | 420 |
| WP_053171320 | 79.8 | Streptomyces virginiae | 449 | 420 |
| WP_030712260 | 79.8 | Streptomyces sp. NRRL S-237 | 449 | 420 |
| WP_033218333 | 79.5 | Streptomyces virginiae | 449 | 420 |
| WP_030850543 | 79.5 | Streptomyces | 450 | 420 |
| WP_030774478 | 79.3 | Streptomyces sp. NRRL F-2664 | 450 | 421 |
| WP_052876505 | 78.9 | Streptomyces sp. NRRL F-4335 | 451 | 422 |
| WP_019886521 | 78.9 | Streptomyces purpureus | 463 | 426 |
| WP_005320871 | 78.0 | Streptomyces pristinaespiralis | 453 | 419 |
| WP_030548298 | 76.6 | Streptomyces albus | 459 | 427 |
| WP_064069271 | 75.5 | Streptomyces albulus | 453 | 421 |
| WP_026277977 | 75.3 | Streptomyces sp. CNT372 | 458 | 417 |

TABLE 8B
List of sequences with percent identity to SspCPro23 full
length protein identified from Genome Quest database

| | | | | Align. |
|---|---|---|---|---|
| GQ Identifier | PID | Organism | Length | length |
| WO2015048332-44360 | 88.3 | Streptomyces venezuelae | 456 | 419 |
| WO2015048332-44127 | 80.4 | Streptomyces sp. C | 455 | 424 |
| WO2015048332-44022 | 78.0 | Streptomyces | 453 | 419 |
| | | pristinaespiralis | | |
| | | ATCC 25486 | | |
| US8076468-0024 | 77.1 | Streptomyces griseus | 255 | 253 |
| EP2205730-0009 | 76.5 | Streptomyces sp.; | 256 | 255 |
| | | Strain 1AG3 | | |

TABLE 9A
List of sequences with percent identity to SspCPro59 full length
protein identified from the NCBI non-redundant protein database

| | | | Sequence | Alignment |
|---|---|---|---|---|
| Accession # | PID | Organism | Length | Length |
| WP_029386953 | 78.1 | Streptomyces leeuwenhoekii | 394 | 392 |
| WP_046250145 | 77.3 | Streptomyces sp. MBT28 | 357 | 357 |
| WP_069630550 | 76.9 | Streptomyces niveus | 444 | 429 |
| WP_031232554 | 76.5 | Streptomyces niveus | 444 | 429 |
| EST18641 | 76.5 | Streptomyces niveus | 459 | 429 |
| | | NCIMB 11891 | | |
| WP_064729342 | 76.3 | Streptomyces parvulus | 457 | 427 |
| WP_047121827 | 75.8 | Streptomyces leeuwenhoekii | 464 | 434 |
| WP_063482838 | 75.7 | Streptomyces ambofaciens | 454 | 419 |
| WP_044383230 | 75.6 | Streptomyces cyaneogriseus | 464 | 434 |
| AJP05780 | 75.6 | Streptomyces cyaneogriseus | 452 | 434 |
| | | subsp. noncyanogenus | | |
| WP_055418378 | 75.5 | Streptomyces pactum | 457 | 429 |
| WP_069979026 | 75.4 | Streptomyces rubrolavendulae | 454 | 427 |
| WP_030027622 | 75.3 | Streptomyces flavotricini | 348 | 352 |
| WP_031135572 | 75.2 | Streptomyces fradiae | 454 | 427 |
| WP_030970235 | 75.1 | Streptomyces sp. NRRL F-4835 | 437 | 426 |
| CAH04620 | 74.9 | Streptomyces fradiae | 454 | 427 |
| WP_019329665 | 74.9 | Streptomyces sp. TOR3209 | 457 | 426 |
| WP_031022018 | 74.9 | Streptomyces sp. NRRL WC-3795 | 457 | 426 |
| WP_053135598 | 74.6 | Streptomyces ambofaciens ATCC | 454 | 426 |
| | | 23877 | | |
| WP_023590970 | 74.5 | Streptomyces thermolilacinus | 455 | 428 |
| | | SPC6 | | |
| WP_059300010 | 74.5 | Streptomyces canus | 455 | 427 |

TABLE 9B
List of sequences with percent identity to SspCPro59 full
length protein identified from Genome Quest database

| | | | | Align. |
|---|---|---|---|---|
| GQ Identifier | PID | Organism | Length | length |
| US8076468-0024 | 79.8 | Streptomyces griseus | 255 | 253 |
| EP2205730-0009 | 76.9 | Streptomyces sp.; | 256 | 255 |
| | | Strain 1AG3 | | |
| WO2015048332-43724 | 75.2 | Streptomyces fradiae | 454 | 427 |
| WO2015048332-43726 | 74.9 | Streptomyces fradiae | 454 | 427 |

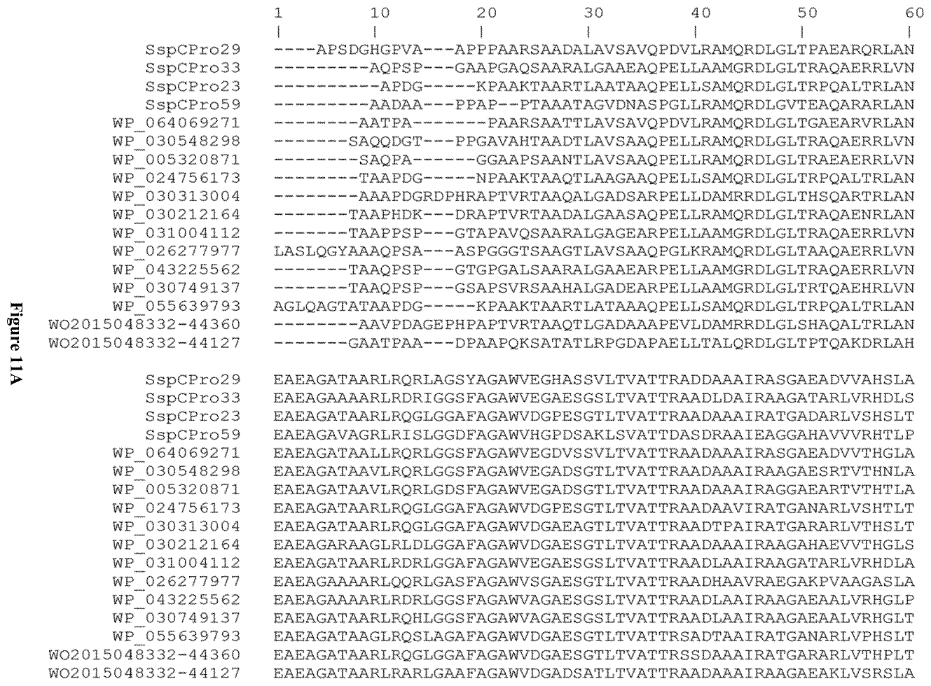

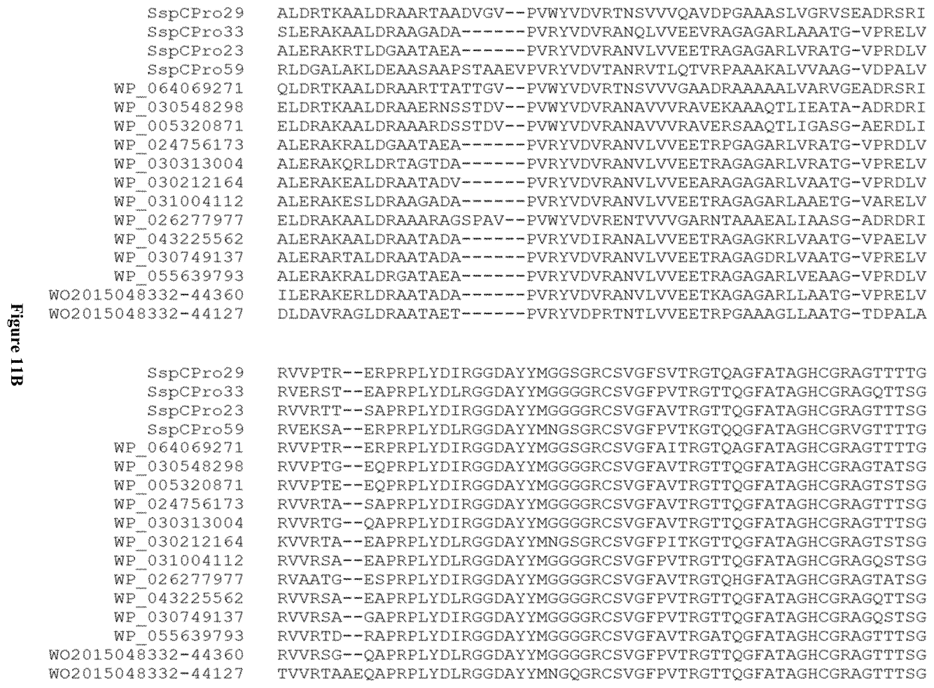

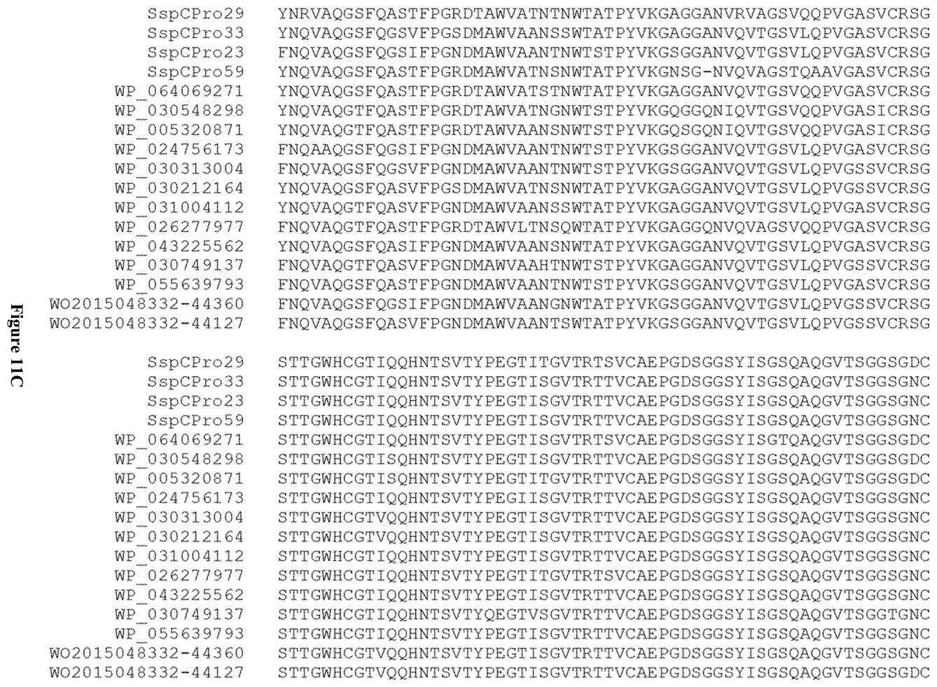

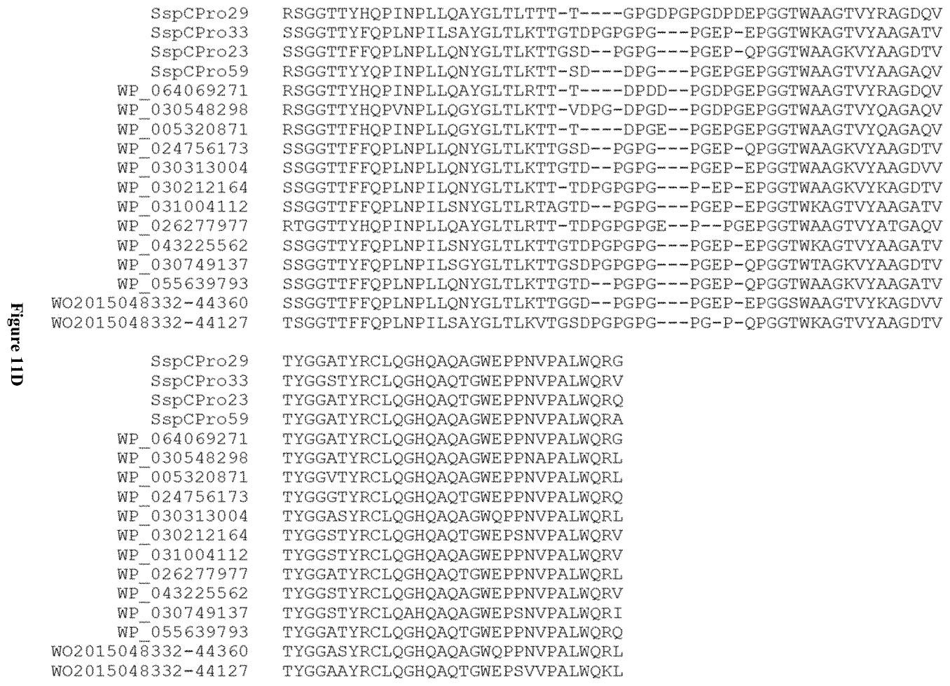

The amino acid sequences for SspCPro29 (SEQ ID NO:22); SspCPro33 (SEQ ID NO:23); SspCPro23 (SEQ ID NO:24); and SspCPro59 (SEQ ID NO:25) and the sequences of other Streptomyces sp serine proteases: WP_064069271 (SEQ ID NO:26); WP_043225562 (SEQ ID NO:27); WP_024756173 (SEQ ID NO:28); WP_030548298 (SEQ ID NO:29); WP_005320871 (SEQ ID NO:30); WP_055639793 (SEQ ID NO:31); WO2015048332-44360 (SEQ ID NO:32); WO2015048332-44127 (SEQ ID NO:33); WP_030313004 (SEQ ID NO:34); WP_030212164 (SEQ ID NO:35); WP_030749137 (SEQ ID NO:36); WP_031004112 (SEQ ID NO:37); and WP_026277977 (SEQ ID NO:38) were aligned with default parameters using the MUSCLE program from Geneious software (Biomatters Ltd.) (Robert C. Edgar. MUSCLE: multiple sequence alignment with high accuracy and high throughput Nucl. Acids Res. (2004) 32 (5): 1792-1797). The multiple sequence alignment for the overlapping regions is shown on FIG. 11.

### Example 13

### Protein Sequence Analysis of Predicted Catalytic Domains of Streptomyces sp Trypsin-Type Serine Proteases

Related proteins were identified by a BLAST search (Altschul et al., Nucleic Acids Res, 25:3389-402, 1997) using the predicted catalytic domain sequences for SspCPro29 (SEQ ID NO:18); SspCPro33 (SEQ ID NO:19); SspCPro23 (SEQ ID NO:20); and SspCPro59 (SEQ ID NO:21) against Public and Genome Quest Patent databases with search parameters set to default values and a subset are shown on Tables 10A and 10B (SspCPro29); Tables 11A and 11B (SspCPro33); Tables 12A and 12B (SspCPro23); and Tables 13A and 13B (SspCPro59) respectively.

TABLE 10A
List of sequences with percent identity to SspCPro29 predicted catalytic
domain identified from the NCBI non-redundant protein database

| | | | Sequence | Alignment |
|---|---|---|---|---|
| Accession # | PID | Organism | Length | Length |
| WP_064069271 | 95.3 | Streptomyces albulus | 453 | 191 |
| WP_026277977 | 91.6 | Streptomyces sp. CNT372 | 458 | 191 |
| WP_030548298 | 88.5 | Streptomyces albus | 459 | 191 |
| WP_044383230 | 88.4 | Streptomyces cyaneogriseus | 464 | 190 |
| AJP05780 | 88.4 | Streptomyces cyaneogriseus subsp. | 452 | 190 |
| | | noncyanogenus | | |
| WP_005320871 | 88.0 | Streptomyces pristinaespiralis | 453 | 191 |
| WP_047121827 | 87.9 | Streptomyces leeuwenhoekii | 464 | 190 |
| WP_029386953 | 87.9 | Streptomyces leeuwenhoekii | 394 | 190 |
| WP_069630550 | 87.4 | Streptomyces niveus | 444 | 191 |
| WP_069979026 | 86.9 | Streptomyces rubrolavendulae | 454 | 191 |
| WP_055639793 | 86.9 | Streptomyces venezuelae | 451 | 191 |
| WP_031003261 | 86.8 | Streptomyces sp. NRRL WC-3773 | 461 | 190 |
| WP_053699044 | 86.8 | Streptomyces sp. NRRL F-5755 | 460 | 190 |
| WP_060732661 | 86.8 | Streptomyces albus subsp. | 460 | 190 |
| | | albus | | |
| WP_030590236 | 86.8 | Streptomyces griseoflavus | 460 | 190 |
| WP_045323790 | 86.7 | Streptomyces sp. NRRL F-4428 | 449 | 188 |
| WP_030027622 | 86.7 | Streptomyces flavotricini | 348 | 188 |
| WP_031135572 | 86.4 | Streptomyces fradiae | 454 | 191 |
| WP_031232554 | 86.4 | Streptomyces niveus | 444 | 191 |
| EST18641 | 86.4 | Streptomyces niveus NCIMB | 459 | 191 |
| | | 11891 | | |
| WP_043225562 | 86.4 | Streptomyces sp. NRRL F-5193 | 456 | 191 |
| WP_053800418 | 86.3 | Streptomyces rimosus subsp. | 460 | 190 |
| | | pseudoverticillatus | | |
| WP_033032149 | 86.3 | Streptomyces rimosus | 460 | 190 |
| WP_031188739 | 86.3 | Streptomyces rimosus subsp. | 460 | 190 |
| | | rimosus | | |
| WP_030639316 | 86.3 | Streptomyces rimosus | 460 | 190 |
| WP_030633274 | 86.3 | Streptomyces rimosus | 460 | 190 |
| WP_030372610 | 86.3 | Streptomyces rimosus | 460 | 190 |
| WP_030659657 | 86.3 | Streptomyces rimosus | 460 | 190 |
| WP_053685358 | 86.2 | Streptomyces sp. XY593 | 451 | 188 |
| CAH04620 | 85.9 | Streptomyces fradiae | 454 | 191 |
| WP_046779091 | 85.9 | Streptomyces yangpuensis | 451 | 191 |
| WP_019886521 | 85.9 | Streptomyces purpureus | 463 | 191 |
| WP_030022977 | 85.8 | Streptomyces monomycini | 461 | 190 |
| WP_003983795 | 85.8 | Streptomyces rimosus subsp. | 460 | 190 |
| | | rimosus | | |
| WP_053632580 | 85.6 | Streptomyces sp. H021 | 451 | 188 |
| WP_053627230 | 85.6 | Streptomyces sp. XY511 | 451 | 188 |
| WP_053634074 | 85.6 | Streptomyces sp. MMG1064 | 451 | 188 |
| WP_053679192 | 85.6 | Streptomyces sp. XY66 | 451 | 188 |
| WP_030896075 | 85.6 | Streptomyces virginiae | 451 | 188 |
| WP_030829885 | 85.6 | Streptomyces sp. NRRL S-104 | 451 | 188 |
| WP_030658602 | 85.6 | Streptomyces sp. H036 | 451 | 188 |
| WP_037919299 | 85.6 | Streptomyces sp. PCS3-D2 | 454 | 188 |
| WP_030850543 | 85.6 | Streptomyces | 450 | 188 |
| WP_055599201 | 85.3 | Streptomyces aureus | 456 | 191 |
| WP_030313004 | 85.3 | Streptomyces flavochromogenes | 456 | 191 |
| WP_030965679 | 85.3 | Streptomyces sp. NRRL S-378 | 449 | 191 |
| WP_024756173 | 85.3 | Streptomyces exfoliatus | 451 | 191 |
| WP_030774478 | 85.1 | Streptomyces sp. NRRL F-2664 | 450 | 188 |
| WP_031153386 | 85.1 | Streptomyces erythrochromogenes | 448 | 188 |
| WP_053705101 | 85.1 | Streptomyces sp. WM6368 | 449 | 188 |
| WP_053171320 | 85.1 | Streptomyces virginiae | 449 | 188 |
| WP_030385747 | 85.1 | Streptomyces sp. NRRL S-241 | 449 | 188 |

TABLE 10B
List of sequences with percent identity to SspCPro29 predicted
catalytic domain identified from Genome Quest database

| | | | | Align. |
|---|---|---|---|---|
| GQ Identifier | PID | Organism | Length | length |
| WO2015048332-44022 | 88.0 | Streptomyces pristinaespiralis | 453 | 191 |
| | | ATCC 25486 | | |
| WO2015048332-43724 | 86.4 | Streptomyces fradiae | 454 | 191 |
| WO2015048332-43726 | 85.9 | Streptomyces fradiae | 454 | 191 |
| WP_043225562 | 97.4 | Streptomyces sp. NRRL F-5193 | 456 | 191 |
| WP_031004112 | 96.3 | Streptomyces sp. NRRL F-5727 | 454 | 190 |
| WP_030498660 | 95.3 | Microtetraspora glauca | 455 | 191 |
| WP_015038204 | 93.2 | Streptomyces venezuelae | 456 | 191 |
| | | ATCC 10712 | | |
| WP_030212164 | 92.7 | Streptomyces bikiniensis | 454 | 191 |
| WP_007266194 | 92.7 | Streptomyces sp. C | 455 | 191 |
| WP_030712260 | 92.6 | Streptomyces sp. NRRL S-237 | 449 | 188 |
| WP_055599201 | 92.1 | Streptomyces aureus | 456 | 191 |
| WP_030749137 | 92.1 | Streptomyces griseus | 456 | 191 |
| WP_030313004 | 92.1 | Streptomyces flavochromogenes | 456 | 191 |
| WP_053705101 | 92.1 | Streptomyces sp. WM6368 | 449 | 191 |
| WP_053171320 | 92.1 | Streptomyces virginiae | 449 | 191 |
| WP_030385747 | 92.1 | Streptomyces sp. NRRL S-241 | 449 | 191 |
| WP_053685358 | 92.1 | Streptomyces sp. XY593 | 451 | 189 |
| WP_024756173 | 91.6 | Streptomyces exfoliatus | 451 | 191 |
| WP_055639793 | 91.6 | Streptomyces venezuelae | 451 | 191 |
| WP_030965679 | 91.6 | Streptomyces sp. NRRL S-378 | 449 | 191 |
| WP_053632580 | 91.5 | Streptomyces sp. H021 | 451 | 189 |
| WP_053627230 | 91.5 | Streptomyces sp. XY511 | 451 | 189 |
| WP_053634074 | 91.5 | Streptomyces sp. MMG1064 | 451 | 189 |
| WP_053679192 | 91.5 | Streptomyces sp. XY66 | 451 | 189 |
| WP_030896075 | 91.5 | Streptomyces virginiae | 451 | 189 |
| WP_030829885 | 91.5 | Streptomyces sp. NRRL S-104 | 451 | 189 |
| WP_030658602 | 91.5 | Streptomyces sp. H036 | 451 | 189 |
| WP_062759972 | 91.1 | Streptomyces sp. WAC04657 | 454 | 191 |
| WP_033218333 | 91.1 | Streptomyces virginiae | 449 | 191 |
| WP_037919299 | 91.1 | Streptomyces sp. PCS3-D2 | 454 | 191 |
| WP_046779091 | 91.1 | Streptomyces yangpuensis | 451 | 191 |
| WP_045323790 | 91.1 | Streptomyces sp. NRRL F-4428 | 449 | 191 |
| WP_030027622 | 91.1 | Streptomyces flavotricini | 348 | 191 |
| WP_030016658 | 91.1 | Streptomyces lavendulae | 369 | 191 |
| WP_030850543 | 91.0 | Streptomyces | 450 | 189 |
| WP_030545445 | 90.6 | Streptomyces exfoliatus | 456 | 191 |
| WP_030208917 | 90.6 | Streptomyces griseoluteus | 456 | 191 |
| WP_033200913 | 90.6 | Streptomyces viridochromogenes | 456 | 191 |
| WP_017236541 | 90.6 | Streptomyces sp. SS | 456 | 191 |
| WP_019886521 | 90.6 | Streptomyces purpureus | 463 | 191 |
| WP_031144485 | 90.6 | Streptomyces xanthophaeus | 447 | 191 |
| WP_052876505 | 90.6 | Streptomyces sp. NRRL F-4335 | 451 | 191 |
| WP_056557852 | 90.1 | Streptomyces sp. Root66D1 | 454 | 191 |
| WP_053644256 | 90.1 | Streptomyces sp. NRRL F-6492 | 455 | 191 |
| WP_031153386 | 89.5 | Streptomyces erythrochromogenes | 448 | 191 |
| WP_030774478 | 89.0 | Streptomyces sp. NRRL F-2664 | 450 | 191 |
| BAU88265 | 88.0 | Streptomyces laurentii | 445 | 191 |
| WP_030548298 | 86.4 | Streptomyces albus | 459 | 191 |
| WP_064069271 | 86.4 | Streptomyces albulus | 453 | 191 |
| WP_047121827 | 86.3 | Streptomyces leeuwenhoekii | 464 | 190 |
| WP_029386953 | 86.3 | Streptomyces leeuwenhoekii | 394 | 190 |
| WP_005320871 | 85.9 | Streptomyces pristinaespiralis | 453 | 191 |
| WP_026277977 | 85.9 | Streptomyces sp. CNT372 | 458 | 191 |
| WP_069630550 | 85.3 | Streptomyces niveus | 444 | 191 |
| WP_064729342 | 85.3 | Streptomyces parvulus | 457 | 191 |
| WP_069979026 | 85.3 | Streptomyces rubrolavendulae | 454 | 191 |
| WP_031135572 | 84.8 | Streptomyces fradiae | 454 | 191 |

TABLE 11B
List of sequences with percent identity to SspCPro33 predicted
catalytic domain identified from Genome Quest database

| | | | | Align. |
|---|---|---|---|---|
| GQ Identifier | PID | Organism | Length | length |
| WO2015048332-44360 | 93.2 | Streptomyces venezuelae | 456 | 191 |
| WO2015048332-44127 | 92.7 | Streptomyces sp. C | 455 | 191 |
| WO2015048332-44022 | 85.9 | Streptomyces | 453 | 191 |
| | | pristinaespiralis ATCC | | |
| | | 25486 | | |
| WO2015048332-43724 | 84.8 | Streptomyces fradiae | 454 | 191 |
| WP_024756173 | 99.0 | Streptomyces exfoliatus | 451 | 191 |
| WP_055599201 | 98.4 | Streptomyces aureus | 456 | 191 |
| WP_030313004 | 98.4 | Streptomyces | 456 | 191 |
| | | flavochromogenes | | |
| WP_055639793 | 98.4 | Streptomyces venezuelae | 451 | 191 |
| WP_015038204 | 96.9 | Streptomyces venezuelae | 456 | 191 |
| | | ATCC 10712 | | |
| WP_030545445 | 95.8 | Streptomyces exfoliatus | 456 | 191 |
| WP_030208917 | 95.3 | Streptomyces griseoluteus | 456 | 191 |
| WP_019886521 | 95.3 | Streptomyces purpureus | 463 | 191 |
| WP_033200913 | 94.8 | Streptomyces | 456 | 191 |
| | | viridochromogenes | | |
| WP_017236541 | 94.2 | Streptomyces sp. SS | 456 | 191 |
| WP_043225562 | 94.2 | Streptomyces sp. NRRL F-5193 | 456 | 191 |
| WP_056557852 | 93.2 | Streptomyces sp. Root66D1 | 454 | 191 |
| WP_053685358 | 93.1 | Streptomyces sp. XY593 | 451 | 189 |
| WP_053632580 | 92.6 | Streptomyces sp. H021 | 451 | 189 |
| WP_053627230 | 92.6 | Streptomyces sp. XY511 | 451 | 189 |
| WP_053634074 | 92.6 | Streptomyces sp. MMG1064 | 451 | 189 |
| WP_053679192 | 92.6 | Streptomyces sp. XY66 | 451 | 189 |
| WP_030896075 | 92.6 | Streptomyces virginiae | 451 | 189 |
| WP_030829885 | 92.6 | Streptomyces sp. NRRL S-104 | 451 | 189 |
| WP_030658602 | 92.6 | Streptomyces sp. H036 | 451 | 189 |
| WP_031004112 | 92.1 | Streptomyces sp. NRRL F-5727 | 454 | 190 |
| WP_030712260 | 92.0 | Streptomyces sp. NRRL S-237 | 449 | 188 |
| WP_053644256 | 91.6 | Streptomyces sp. NRRL F-6492 | 455 | 191 |
| WP_030749137 | 91.6 | Streptomyces griseus | 456 | 191 |
| WP_007266194 | 91.6 | Streptomyces sp. C | 455 | 191 |
| WP_053705101 | 91.6 | Streptomyces sp. WM6368 | 449 | 191 |
| WP_053171320 | 91.6 | Streptomyces virginiae | 449 | 191 |
| WP_030965679 | 91.6 | Streptomyces sp. NRRL S-378 | 449 | 191 |
| WP_030385747 | 91.6 | Streptomyces sp. NRRL S-241 | 449 | 191 |
| WP_037919299 | 91.6 | Streptomyces sp. PCS3-D2 | 454 | 191 |
| WP_030498660 | 91.1 | Microtetraspora glauca | 455 | 191 |
| WP_030212164 | 90.6 | Streptomyces bikiniensis | 454 | 191 |
| WP_033218333 | 90.6 | Streptomyces virginiae | 449 | 191 |
| WP_046779091 | 90.6 | Streptomyces yangpuensis | 451 | 191 |
| WP_030850543 | 90.5 | Streptomyces | 450 | 189 |
| WP_062759972 | 90.1 | Streptomyces sp. WAC04657 | 454 | 191 |
| WP_052876505 | 90.1 | Streptomyces sp. NRRL F-4335 | 451 | 191 |
| WP_045323790 | 90.1 | Streptomyces sp. NRRL F-4428 | 449 | 191 |
| WP_031153386 | 90.1 | Streptomyces erythrochromogenes | 448 | 191 |
| WP_030027622 | 90.1 | Streptomyces flavotricini | 348 | 191 |
| WP_030016658 | 90.1 | Streptomyces lavendulae | 369 | 191 |
| WP_031144485 | 89.5 | Streptomyces xanthophaeus | 447 | 191 |
| BAU88265 | 89.5 | Streptomyces laurentii | 445 | 191 |
| WP_030774478 | 89.0 | Streptomyces sp. NRRL F-2664 | 450 | 191 |
| WP_005320871 | 89.0 | Streptomyces pristinaespiralis | 453 | 191 |
| WP_030548298 | 89.0 | Streptomyces albus | 459 | 191 |
| WP_069630550 | 89.0 | Streptomyces niveus | 444 | 191 |
| WP_031232554 | 88.0 | Streptomyces niveus | 444 | 191 |
| EST18641 | 88.0 | Streptomyces niveus NCIMB 11891 | 459 | 191 |
| WP_064069271 | 87.4 | Streptomyces albulus | 453 | 191 |
| WP_069979026 | 87.4 | Streptomyces rubrolavendulae | 454 | 191 |
| WP_031135572 | 86.9 | Streptomyces fradiae | 454 | 191 |
| WP_026277977 | 86.4 | Streptomyces sp. CNT372 | 458 | 191 |
| CAH04620 | 86.4 | Streptomyces fradiae | 454 | 191 |
| WP_030659657 | 85.3 | Streptomyces rimosus | 460 | 190 |
| WP_047121827 | 84.7 | Streptomyces leeuwenhoekii | 464 | 190 |
| WP_029386953 | 84.7 | Streptomyces leeuwenhoekii | 394 | 190 |
| WP_053699044 | 84.7 | Streptomyces sp. NRRL F-5755 | 460 | 190 |
| WP_060732661 | 84.7 | Streptomyces albus subsp. | 460 | 190 |
| | | albus | | |
| WP_030590236 | 84.7 | Streptomyces griseoflavus | 460 | 190 |

TABLE 12B
List of sequences with percent identity to SspCPro23 predicted
catalytic domain identified from Genome Quest database

| | | | | Align. |
|---|---|---|---|---|
| GQ Identifier | PID | Organism | Length | length |
| WO2015048332-44360 | 96.9 | Streptomyces venezuelae | 456 | 191 |
| WO2015048332-44127 | 91.6 | Streptomyces sp. C | 455 | 191 |
| WO2015048332-44022 | 89.0 | Streptomyces pristinaespiralis | 453 | 191 |
| | | ATCC 25486 | | |
| WO2015048332-43724 | 86.9 | Streptomyces fradiae | 454 | 191 |
| WO2015048332-43726 | 86.4 | Streptomyces fradiae | 454 | 191 |

TABLE 13A
List of sequences with percent identity to SspCPro59 predicted catalytic
domain identified from the NCBI non-redundant protein database

| | | | Sequence | Alignment |
|---|---|---|---|---|
| Accession # | PID | Organism | Length | Length |
| WP_047121827 | 90.5 | Streptomyces leeuwenhoekii | 464 | 190 |
| WP_029386953 | 90.5 | Streptomyces leeuwenhoekii | 394 | 190 |
| WP_069630550 | 89.5 | Streptomyces niveus | 444 | 191 |
| WP_044383230 | 89.5 | Streptomyces cyanogriseus | 464 | 190 |
| AJP05780 | 89.5 | Streptomyces cyanogriseus subsp. | 452 | 190 |
| | | noncyanogenus | | |
| WP_053800418 | 88.9 | Streptomyces rimosus subsp. | 460 | 190 |
| | | pseudoverticillatus | | |
| WP_033032149 | 88.9 | Streptomyces rimosus | 460 | 190 |
| WP_031188739 | 88.9 | Streptomyces rimosus subsp. | 460 | 190 |
| | | rimosus | | |
| WP_030639316 | 88.9 | Streptomyces rimosus | 460 | 190 |
| WP_030633274 | 88.9 | Streptomyces rimosus | 460 | 190 |
| WP_030212164 | 88.5 | Streptomyces bikiniensis | 454 | 191 |
| WP_031232554 | 88.5 | Streptomyces niveus | 444 | 191 |
| EST18641 | 88.5 | Streptomyces niveus NCIMB 11891 | 459 | 191 |
| WP_030372610 | 88.4 | Streptomyces rimosus | 460 | 190 |
| WP_063482838 | 88.0 | Streptomyces ambofaciens | 454 | 191 |
| WP_053135598 | 88.0 | Streptomyces ambofaciens ATCC 23877 | 454 | 191 |
| WP_064069271 | 88.0 | Streptomyces albulus | 453 | 191 |
| WP_043225562 | 88.0 | Streptomyces sp. NRRL F-5193 | 456 | 191 |
| WP_031003261 | 87.9 | Streptomyces sp. NRRL WC-3773 | 461 | 190 |
| WP_003983795 | 87.9 | Streptomyces rimosus subsp. | 460 | 190 |
| | | rimosus | | |
| WP_053699044 | 87.9 | Streptomyces sp. NRRL F-5755 | 460 | 190 |
| WP_062759972 | 87.4 | Streptomyces sp. WAC04657 | 454 | 191 |
| WP_064729342 | 87.4 | Streptomyces parvulus | 457 | 191 |
| WP_060732661 | 87.4 | Streptomyces albus subsp. | 460 | 190 |
| | | albus | | |
| WP_030590236 | 87.4 | Streptomyces griseoflavus | 460 | 190 |
| WP_053627230 | 87.3 | Streptomyces sp. XY511 | 451 | 189 |
| WP_053685358 | 87.3 | Streptomyces sp. XY593 | 451 | 189 |
| WP_030658602 | 87.3 | Streptomyces sp. H036 | 451 | 189 |
| WP_053644256 | 86.9 | Streptomyces sp. NRRL F-6492 | 455 | 191 |
| WP_046250145 | 86.9 | Streptomyces sp. MBT28 | 357 | 191 |
| WP_031022018 | 86.9 | Streptomyces sp. NRRL WC-3795 | 457 | 191 |
| WP_030970235 | 86.9 | Streptomyces sp. NRRL F-4835 | 437 | 191 |
| WP_055418378 | 86.9 | Streptomyces pactum | 457 | 191 |
| WP_069979026 | 86.9 | Streptomyces rubrolavendulae | 454 | 191 |
| WP_030965679 | 86.9 | Streptomyces sp. NRRL S-378 | 449 | 191 |
| WP_030022977 | 86.8 | Streptomyces monomycini | 461 | 190 |
| WP_030659657 | 86.8 | Streptomyces rimosus | 460 | 190 |
| WP_053632580 | 86.8 | Streptomyces sp. H021 | 451 | 189 |
| WP_053634074 | 86.8 | Streptomyces sp. MMG1064 | 451 | 189 |
| WP_053679192 | 86.8 | Streptomyces sp. XY66 | 451 | 189 |
| WP_030896075 | 86.8 | Streptomyces virginiae | 451 | 189 |
| WP_030829885 | 86.8 | Streptomyces sp. NRRL S-104 | 451 | 189 |
| WP_019329665 | 86.4 | Streptomyces sp. TOR3209 | 457 | 191 |
| WP_015038204 | 86.4 | Streptomyces venezuelae ATCC | 456 | 191 |
| | | 10712 | | |
| WP_055639793 | 86.4 | Streptomyces venezuelae | 451 | 191 |
| WP_037919299 | 86.4 | Streptomyces sp. PCS3-D2 | 454 | 191 |
| WP_031135572 | 86.4 | Streptomyces fradiae | 454 | 191 |
| WP_046779091 | 86.4 | Streptomyces yangpuensis | 451 | 191 |
| WP_026277977 | 86.4 | Streptomyces sp. CNT372 | 458 | 191 |
| WP_043506163 | 85.9 | Streptomyces glaucescens | 442 | 191 |
| WP_037929773 | 85.9 | Streptomyces toyocaensis | 435 | 191 |
| AIR96443 | 85.9 | Streptomyces glaucescens | 457 | 191 |
| KES08095 | 85.9 | Streptomyces toyocaensis | 457 | 191 |
| WP_030548298 | 85.9 | Streptomyces albus | 459 | 191 |
| WP_005320871 | 85.9 | Streptomyces pristinaespiralis | 453 | 191 |
| WP_031144485 | 85.9 | Streptomyces xanthophaeus | 447 | 191 |
| CAH04620 | 85.9 | Streptomyces fradiae | 454 | 191 |
| WP_031153386 | 85.9 | Streptomyces erythrochromogenes | 448 | 191 |
| WP_045323790 | 85.9 | Streptomyces sp. NRRL F-4428 | 449 | 191 |
| WP_030027622 | 85.9 | Streptomyces flavotricini | 348 | 191 |
| WP_023590970 | 85.9 | Streptomyces | 455 | 191 |
| | | thermolilacinus SPC6 | | |
| WP_055569787 | 85.9 | Streptomyces atriruber | 455 | 191 |
| WP_069884582 | 85.9 | Streptomyces luteocolor | 456 | 191 |
| WP_055698079 | 85.9 | Streptomyces silaceus | 456 | 191 |
| WP_059300010 | 85.9 | Streptomyces canus | 455 | 191 |
| WP_039831526 | 85.8 | Streptomyces viridosporus | 442 | 190 |
| WP_050793881 | 85.8 | Streptomyces ghanaensis | 439 | 190 |
| EFE67698 | 85.8 | Streptomyces ghanaensis ATCC | 461 | 190 |
| | | 14672 | | |
| WP_018959758 | 85.7 | Streptomyces sp. CNB091 | 459 | 189 |
| WP_030712260 | 85.6 | Streptomyces sp. NRRL S-237 | 449 | 188 |
| WP_058941217 | 85.3 | Streptomyces kanasensis | 459 | 191 |
| WP_053705101 | 85.3 | Streptomyces sp. WM6368 | 449 | 191 |
| WP_053171320 | 85.3 | Streptomyces virginiae | 449 | 191 |
| WP_030385747 | 85.3 | Streptomyces sp. NRRL S-241 | 449 | 191 |
| WP_037835235 | 85.3 | Streptomyces sp. NRRL F-5650 | 447 | 191 |
| WP_030793011 | 85.3 | Streptomyces sp. NRRL S-920 | 459 | 191 |
| WP_031004112 | 85.3 | Streptomyces sp. NRRL F-5727 | 454 | 190 |
| WP_030850543 | 85.2 | Streptomyces | 450 | 189 |
| WP_055599201 | 84.8 | Streptomyces aureus | 456 | 191 |
| WP_030313004 | 84.8 | Streptomyces flavochromogenes | 456 | 191 |
| WP_019886521 | 84.8 | Streptomyces purpureus | 463 | 191 |
| WP_024756173 | 84.8 | Streptomyces exfoliatus | 451 | 191 |
| WP_033218333 | 84.8 | Streptomyces virginiae | 449 | 191 |
| WP_053913363 | 84.8 | Streptomyces sp. TP-A0875 | 457 | 191 |
| WP_051821392 | 84.8 | Streptomyces sp. NRRL F-5065 | 457 | 191 |
| WP_030016658 | 84.8 | Streptomyces lavendulae | 369 | 191 |

TABLE 13B
List of sequences with percent identity to SspCPro59 predicted
catalytic domain identified from Genome Quest database

| | | | | Align. |
|---|---|---|---|---|
| GQ Identifier | PID | Organism | Length | length |
| WO2015048332-43724 | 86.4 | Streptomyces fradiae | 454 | 191 |
| WO2015048332-44360 | 86.4 | Streptomyces venezuelae | 456 | 191 |
| WO2015048332-43726 | 85.9 | Streptomyces fradiae | 454 | 191 |
| WO2015048332-44022 | 85.9 | Streptomyces pristinaespiralis | 453 | 191 |
| | | ATCC 25486 | | |
| WO2015048332-43751 | 85.8 | Streptomyces ghanaensis | 461 | 190 |
| | | ATCC 14672 | | |
| WO2015048332-44127 | 84.3 | Streptomyces sp. C | 455 | 191 |
| US8535927-0035 | 84.2 | Streptomyces griseus | 195 | 190 |
| US8076468-0024 | 84.2 | Streptomyces griseus | 255 | 190 |
| WO2015048332-44248 | 84.2 | Streptomyces sp. W007 | 457 | 190 |
| WO2015048332-43810 | 84.2 | Streptomyces griseus | 457 | 190 |
| WO2015048332-43844 | 84.2 | Streptomyces griseus | 457 | 190 |
| US8076468-0023 | 84.2 | Streptomyces griseus | 457 | 190 |
| WO2015048332-43602 | 83.3 | Streptomyces coelicoflavus | 355 | 191 |
| | | ZG0656 | | |
| WO2015048332-44050 | 83.2 | Streptomyces roseosporus | 455 | 190 |
| | | NRRL 15998 | | |
| WO2015048332-43682 | 82.1 | Streptomyces davawensis | 450 | 190 |
| | | JCM 4913 | | |
| WO2015048332-43645 | 81.7 | Streptomyces coelicolor | 463 | 191 |
| WO2015048332-43956 | 81.7 | Streptomyces lividans | 358 | 191 |
| | | TK24 | | |
| US8535927-0036 | 81.7 | Streptomyces coelicolor | 197 | 191 |
| WO2015048332-43953 | 81.2 | Streptomyces lividans | 458 | 191 |
| WO2015048332-44149 | 80.5 | Streptomyces sp. e14 | 457 | 190 |
| US8076468-0009 | 80.1 | Streptomyces sp. | 256 | 191 |
| US8076468-0003 | 80.1 | Streptomyces sp. | 453 | 191 |
| US8076468-0011 | 80.1 | Streptomyces sp. | 428 | 191 |
| WO2005052161-0649 | 79.9 | Streptomyces | 381 | 189 |
| WO2015048332-44081 | 79.9 | Streptomyces sp. | 382 | 189 |
| WO2005052146-0038 | 79.9 | Streptomyces sp. | 187 | 189 |
| WO2015048332-43913 | 79.0 | Streptomyces hygroscopicus | 439 | 190 |
| WO2015048332-44186 | 78.4 | Streptomyces sp. | 449 | 190 |
| | | SirexAA-E | | |
| WO2015048332-44289 | 77.0 | Streptomyces sviceus | 454 | 191 |
| | | ATCC 29083 | | |
| WO2015048332-44213 | 76.8 | Streptomyces sp. SM8 | 453 | 190 |
| WO2015048332-43423 | 76.8 | Streptomyces albus | 453 | 190 |
| | | J1074 | | |
| WO2015048332-43766 | 76.3 | Streptomyces griseoaurantiacus | 449 | 190 |
| | | M045 | | |

An alignment of the predicted catalytic domain sequences of SspCPro29 (SEQ ID NO: 18; aa 213-403 of SEQ ID NO:3); SspCPro33 (SEQ ID NO: 19, aa 204-394 of SEQ ID NO:6); SspCPro23 (SEQ ID NO: 20, aa 201-391 of SEQ ID NO:9); SspCPro59 (SEQ ID NO: 21, aa 206-395 of SEQ ID NO:12); WP_064069271 (SEQ ID NO:42, aa 204-394 of SEQ ID NO:26); WP_043225562 (SEQ ID NO:43, aa 204-394 of SEQ ID NO:27); WP_024756173 (SEQ ID NO:44, aa 201-391 of SEQ ID NO:28); WP_030548298 (SEQ ID NO:45, aa 207-397 of SEQ ID NO:29); WP_005320871 (SEQ ID NO:46, aa 204-394 of SEQ ID NO:30); amino acid residues 138-328 of WP_029386953 (SEQ ID NO:47); WP_026277977 (SEQ ID NO:48, aa 207-397 of SEQ ID NO:38); amino acid residues 208-398 of WP_044383230 (SEQ ID NO:49); amino acid residues 193-383 of WP_069630550 (SEQ ID NO:50); WP_055639793 (SEQ ID NO:51,aa 201-391 of SEQ ID NO:31); amino acid residues of 211-401 of WP_053699044 (SEQ ID NO:52); amino acid residues 205-395 of WP_031135572 (SEQ ID NO:53) was performed as described above and is shown in FIG. 12. The predicted catalytic domain consensus sequence from FIG. 12 is set forth as SEQ ID NO:54. For positions in consensus sequences were multiple amino acids are considered, they are depicted using X=I or L and the IUPAC codes: B=D or N.

What is claimed is:

1. An isolated polypeptide having serine protease activity, selected from:
a) a polypeptide comprising an amino acid sequence with at least 91% identity with the amino acid sequence of SEQ ID NO:22;
b) a polypeptide comprising an amino acid sequence with at least 94% identity with the amino acid sequence of SEQ ID NO:23;
c) a polypeptide comprising an amino acid sequence with at least 98% identity with the amino acid sequence of SEQ ID NO:24; and
d) a polypeptide comprising an amino acid sequence with at least 80% identity with the amino acid sequence of SEQ ID NO:25.

2. An isolated polypeptide having serine protease activity and comprising a predicted precursor amino acid sequence selected from: SEQ ID NO:3; SEQ ID NO:6; SEQ ID NO:9; and SEQ ID NO:12.

3. An isolated polypeptide having serine protease activity and comprising a protease catalytic region selected from:
a) an amino acid sequence with at least 96% identity with the amino acid sequence of SEQ ID NO:18;
b) an amino acid sequence with at least 98% identity with the amino acid sequence of SEQ ID NO:19;
c) an amino acid sequence of SEQ ID NO:20; and
d) an amino acid sequence with at least 91% identity with the amino acid sequence of SEQ ID NO:21.

4. A recombinant construct comprising a regulatory sequence functional in a production host operably linked to a nucleotide sequence encoding at least one polypeptide of any one of claims 1-3

5. The recombinant construct of claim 4, wherein said host is selected from the group consisting of fungi, bacteria, and algae.

6. A method for producing at least one polypeptide comprising:
(a) transforming a production host with the recombinant construct of claim 4; and
(b) culturing the production host of step (a) under conditions whereby at least one polypeptide is produced.

7. A method according to claim 6, wherein the polypeptide is optionally recovered from the production host.

8. A serine protease-containing culture supernatant obtained by the method of claim 6 or 7.

9. A recombinant microbial production host for expressing at least one polypeptide, said recombinant microbial production host comprising the recombinant construct of claim 4.

10. A production host according to claim 9, wherein said host is selected from the group consisting of bacteria, fungi and algae.

11. Animal feed comprising at least one polypeptide of any one of claims 1-3, wherein said polypeptide is present in an amount from 1-20 g/ton feed.

12. The animal feed of claim 11 further comprising: a) at least one direct fed microbial or b) at least one other enzyme or c) at least one direct fed microbial and at least one other enzyme.

13. A feed, feedstuff, a feed additive composition or premix comprising at least one polypeptide having serine protease activity of any of claims 1-3.

14. The feed, feedstuff, feed additive composition or premix of claim 13 further comprising:
a) at least one direct fed microbial or b) at least one other enzyme or c) at least one direct fed microbial and at least one other enzyme.

15. The feed additive composition of claim of any of claims 13-14 wherein said composition further comprises at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabisulfite, methyl paraben and propyl paraben.

16. A granulated feed additive composition for use in animal feed comprising the serine protease polypeptide of any of claims 1-3, wherein the granulated feed additive composition comprises particles produced by a process selected from the group consisting of high shear granulation, drum granulation, extrusion, spheronization, fluidized bed agglomeration, fluidized bed spray coating, spray drying, freeze drying, prilling, spray chilling, spinning disk atomization, coacervation, tableting, or any combination of the above processes.

17. The granulated feed additive composition of claim 16, wherein the mean diameter of the particles is greater than 50 microns and less than 2000 microns.

18. The feed additive composition of claim 17 wherein said composition is in a liquid form.

19. The feed additive composition of claim 18 wherein said composition is in a liquid form suitable for spray-drying on a feed pellet.


SEQUENCE LISTING (54 sequences)

SEQ ID NO: 1 - DNA, 1395 bp, Streptomyces sp. C009
atgttccaccgacacgccagagccgcgtgcgtgggcgccgtgaccgtgggcgcgctggtcctgacggcactgcaaggggccgctgcggcgccctcggacggccacggcccggtcgccgcgcccccaccggcggcccgttcggcggccgacgccctcgccgtgtccgccgtacagccggacgtgctgcgtgccatgcaacgggacctcggcctcactccggccgaggcccggcagcggctggcgaacgaggcggaggcgggcgcgaccgccgcccgtctccggcagcggctggccggctcgtacgccggtgcctgggtcgaggggcacgcgtcgtccgtcctgaccgtcgccaccacccgggccgatgacgcggcggcgatcagggcgagcggcgccgaggccgatgtcgtcgcgcacagtctggcggcgctcgaccgcaccaaggcggctctggaccgtgcggcccgcaccgccgccgacgtcggcgtccccgtctggtacgtcgacgtccgcaccaactccgtggtggtccaggccgtggacccgggggcggcggcgtcgctggtcggccgggtgagcgaggccgaccggtcgcggatacgggtcgtcccgacgcgggaacggccgcgcccgctgtacgacatcaggggcggtgacgcgtactacatgggcggctccggccgctgctcggtgggcttctcggtcacccgtgggacgcaggcgggcttcgcgacggcggggcactgcggccgggccggcacgaccacgaccgggtacaaccgggtggcgcagggctcgttccaggcctcgacgttcccgggccgggacacggcctgggtggcgacgaacaccaactggaccgcgaccccgtacgtgaagggcgcgggcggcgcgaacgtccgggtggccggttcggtccagcagccggtcggcgcctcggtctgccgctcgggctccaccacgggctggcactgcggcaccatccagcagcacaacaccagcgtgacgtacccggagggcaccatcaccggggtgacgcggacctcggtgtgcgccgaacccggtgactccggtgggtcgtacatctccggcagccaggcgcagggcgtcacctccggcggttcgggcgactgccggagcgggggtacgacctaccaccagccgatcaacccgctgctgcaggcgtacgggctgacgctgacgacgaccaccggacccggcgacccgggcccgggcgacccggacgagccgggcggcacatgggcggccggcaccgtctaccgggcgggtgaccaggtcacgtacggcggtgcgacctaccgctgcctgcagggccaccaggcgcaggccgggtgggaaccgccgaacgtcccggcgctctggcagcgcggctga

SEQ ID NO: 2 - protein, 29 aa, Streptomyces sp. C009
MFHRHARAACVGAVTVGALVLTALQGAAA

SEQ ID NO: 3 - protein, 464 aa, Streptomyces sp. C009
MFHRHARAACVGAVTVGALVLTALQGAAAAPSDGHGPVAAPPPAARSAADALAVSAVQPDVLRAMQRDLGLTPAEARQRLANEAEAGATAARLRQRLAGSYAGAWVEGHASSVLTVATTRADDAAAIRASGAEADVVAHSLAALDRTKAALDRAARTAADVGVPVWYVDVRTNSVVVQAVDPGAAASLVGRVSEADRSRIRVVPTRERPRPLYDIRGGDAYYMGGSGRCSVGFSVTRGTQAGFATAGHCGRAGTTTTGYNRVAQGSFQASTFPGRDTAWVATNTNWTATPYVKGAGGANVRVAGSVQQPVGASVCRSGSTTGWHCGTIQQHNTSVTYPEGTITGVTRTSVCAEPGDSGGSYISGSQAQGVTSGGSGDCRSGGTTYHQPINPLLQAYGLTLTTTTGPGDPGPGDPDEPGGTWAAGTVYRAGDQVTYGGATYRCLQGHQAQAGWEPPNVPALWQRG

SEQ ID NO: 4 - DNA, 1371 bp, Streptomyces sp. C001
atgctccgacgacacgcccgtgcggcgtgtaccgcgctggccgccgccgggatggtgctggccgggcttcaggccgggtccgccaccgccgcgcagccctcccccggagccgcgcccggcgcgcagagcgccgctcgggcgctcggcgccgccgaggcgcagcccgaactgctcgccgcgatgggccgcgacctgggcctgacccgggcccaggcagagcgccggctcgtcaacgaggccgaggcgggcgccgcggccgcccggctccgcgaccggatcggcggctccttcgccggggcctgggtcgagggggccgagtccggctccctgaccgtggccaccacccgcgccgccgacctcgacgcgatccgggcggccggggcgacggcccggctcgtccggcacgacctgtcctcgctggagcgggcgaaggccgccctcgaccgggccgcgggcgccgacgcgccggtgcggtacgtggacgtgcgcgccaaccagttggtcgtcgaggaggtccgggccggtgccggggcgcgcctggcggcggccaccggggtgccgcgggagctggtgcgggtggagcggagcaccgaggcgccgcgcccgctgtacgacctccggggcggcgacgcgtactacatgggcggcggcgggcgctgctcggtcggcttcccggtgacccggggcaccacgcagggcttcgcgacggccgggcactgcggccgcgccggccagaccaccagcggctacaaccaggtcgcccagggcagcttccagggctcggtcttccccggcagcgacatggcgtgggtcgcggccaacagcagctggaccgccaccccgtacgtgaagggcgcgggcggcgcgaacgtccaggtcaccggctccgtgctgcagcccgtgggcgcctccgtctgccgctcgggctccaccaccggctggcactgcggcaccatccagcagcacaacacgagcgtgacgtacccggagggcacgatctccggcgtgacccggacgacggtctgcgccgagcccggcgactcgggcggctcgtacatctccggcagccaggcgcagggcgtcacctcgggcggctccggcaactgctccagcggcggcaccacgtacttccagccgctgaacccgatcctgtcggcctacggcctgaccctgaagaccaccggcaccgacccgggccccggcccgggcccgggcgagccggagccgggcggcacgtggaaggcgggcacggtgtacgcggccggcgccaccgtcacgtacggcggttccacctaccgctgcctccagggccaccaggcgcagaccggctgggagccgccgaacgtaccggcgctctggcagcgggtctga

SEQ ID NO: 5 - protein, 30 aa, Streptomyces sp. C001
MLRRHARAACTALAAAGMVLAGLQAGSATA

SEQ ID NO: 6 - protein, 456 aa, Streptomyces sp. C001
MLRRHARAACTALAAAGMVLAGLQAGSATAAQPSPGAAPGAQSAARALGAAEAQPELLAAMGRDLGLTRAQAERRLVNEAEAGAAAARLRDRIGGSFAGAWVEGAESGSLTVATTRAADLDAIRAAGATARLVRHDLSSLERAKAALDRAAGADAPVRYVDVRANQLVVEEVRAGAGARLAAATGVPRELVRVERSTEAPRPLYDLRGGDAYYMGGGGRCSVGFPVTRGTTQGFATAGHCGRAGQTTSGYNQVAQGSFQGSVFPGSDMAWVAANSSWTATPYVKGAGGANVQVTGSVLQPVGASVCRSGSTTGWHCGTIQQHNTSVTYPEGTISGVTRTTVCAEPGDSGGSYISGSQAQGVTSGGSGNCSSGGTTYFQPLNPILSAYGLTLKTTGTDPGPGPGPGEPEPGGTWKAGTVYAAGATVTYGGSTYRCLQGHQAQTGWEPPNVPALWQRV

SEQ ID NO: 7 - DNA, 1356 bp, Streptomyces sp. C003
atgctccgacgacacgcccgcgcggcgtgtaccgcgctggtcgccgccgggatggcgctggccggcctccaggccggcaccgccaccgcggcaccggacgggaaaccggcagcgaagaccgccgctcggaccctcgccgccaccgccgcccagcccgaactcctctccgccatgcagcgcgacctgggactcacccgcccccaggcactgacccggctcgccaacgaggccgaggccggcgccaccgccgcccggctccgccagggcctcggcggcgccttcgccggagcctgggtcgacgggcccgagtccggcaccctgaccgtggccaccacccgcgccgccgacgcggccgcgatacgcgccaccggcgccgacgcccggctcgtctcccacagcctgaccgccctggagcgggccaagcggaccctggacggggccgccaccgccgaggcacccgtccgctacgtcgacgtccgcgccaacgtcctcgtcgtcgaggagacgcgggccggcgccggggcacggctcgtccgggcgaccggggtcccgcgcgacctggtgcgcgtggtcaggaccacatccgcaccgcgcccgctgtacgacatccggggcggcgacgcgtactacatgggcggcggcggccgctgctccgtcggtttcgcggtgacccgcggcacgacgcagggcttcgccactgcgggccactgcggccgcgccggcacgacgaccagcggcttcaaccaggtcgcccagggcagcttccagggctcgatcttccccggcaacgacatggcctgggtcgccgccaacaccaactggacctccaccccgtacgtcaagggctccggcggcgcgaacgtccaggtgaccgggtccgtcctccagcccgtcggcgcgtccgtctgccgctcaggctcgaccaccggctggcactgcggcacgatccagcagcacaacaccagcgtcacctaccccgagggcaccatctccggagtgacccgcacgacggtctgcgccgaacccggcgactccggcggctcgtacatctccggcagccaggcccagggcgtgacctcgggcggctcgggcaactgctccagcggcggcacgaccttcttccagccgctcaacccgctgctgcagaactacggcctgaccctgaagaccaccggcagcgatccgggccccgggcccggcgagccgcagcccggcggcacctgggcggcgggcaaggtctacgcggccggtgacacggtcacctacggcggcgcgacctaccgctgcctccagggccaccaggcccagaccggctgggagccgccgaacgtcccggccctctggcagcgccagtga

SEQ ID NO: 8 - protein, 30 aa, Streptomyces sp. C003
MLRRHARAACTALVAAGMALAGLQAGTATA

SEQ ID NO: 9 - protein, 451 aa, Streptomyces sp. C003
MLRRHARAACTALVAAGMALAGLQAGTATAAPDGKPAAKTAARTLAATAAQPELLSAMQRDLGLTRPQALTRLANEAEAGATAARLRQGLGGAFAGAWVDGPESGTLTVATTRAADAAAIRATGADARLVSHSLTALERAKRTLDGAATAEAPVRYVDVRANVLVVEETRAGAGARLVRATGVPRDLVRVVRTTSAPRPLYDIRGGDAYYMGGGGRCSVGFAVTRGTTQGFATAGHCGRAGTTTSGFNQVAQGSFQGSIFPGNDMAWVAANTNWTSTPYVKGSGGANVQVTGSVLQPVGASVCRSGSTTGWHCGTIQQHNTSVTYPEGTISGVTRTTVCAEPGDSGGSYISGSQAQGVTSGGSGNCSSGGTTFFQPLNPLLQNYGLTLKTTGSDPGPGPGEPQPGGTWAAGKVYAAGDTVTYGGATYRCLQGHQAQTGWEPPNVPALWQRQ

SEQ ID NO: 10 - DNA, 1365 bp, Streptomyces sp. S055
atgctccacagacatgccaaagcgtcctgtgccgcgttcgcggccgtcggcgcgctggtgctggcagggctgcccgcaacggccgccgcggacgcggccccacccgcaccgcccaccgcggccgcgacggcgggcgtcgacaacgcctcgccagggctgctgcgggccatgcagcgcgacctcggagtgaccgaggcgcaggcaagggcgcggctggcgaatgaggccgaggccggtgcggtcgccggacggctgcggatatcgctcggcggcgacttcgccggtgcctgggtgcacggccccgactccgccaaactgagcgtggccaccacggacgcctcggacagggccgcgatcgaggccgggggcgcgcacgccgtcgtcgtacggcacaccctgccccggctggacggagcgctggcgaagctggacgaggcggcgtccgccgctccgtccacggccgccgaggtacccgtccggtacgtggacgtcacggcgaaccgcgtgacgctgcaaaccgtacggccggccgccgcgaaggcgctcgtcgtggccgcgggggtcgaccccgcgctcgtccgcgtggagaagtcggcggagcgtccgcgtccgctgtacgacctgcggggcggcgacgcgtactacatgaacggcagcggccgctgttccgtcggatttccggtcaccaagggaacgcagcagggcttcgccaccgccggacactgcgggcgcgtcggcacgacgaccaccggctacaaccaggtcgcccaggggtccttccaggcctccaccttccccggccgtgacatggcctgggtggccaccaacagcaactggaccgccaccccctacgtcaagggcaacagcggaaacgtccaggtcgccggctcgacacaggccgccgtcggcgcgtcggtctgccgttcggggtccaccaccggctggcactgcggcaccatccagcagcacaacaccagcgtcacctaccccgaaggcaccatcagtggtgtgacccgcaccacggtctgtgccgagcccggcgactcaggcggctcctacatctccggcagccaggcccagggcgtcacctccggcgggtcgggcaactgccgctccggcggcaccacctactaccagccgatcaacccgctgctccagaactacgggctgaccctcaagaccacctcggacgaccccggcccgggtgagccgggggagcccggcggcacctgggccgcgggaaccgtctacgcggcgggagcccaggtgacgtacggcggggccacctaccggtgcctccagggacaccaggcccaggccggctgggagcccccgaacgtgcccgccctgtggcagcgggcgtga

SEQ ID NO: 11 - protein, 28 aa, Streptomyces sp. S055
MLHRHAKASCAAFAAVGALVLAGLPATA

SEQ ID NO: 12 - protein, 454 aa, Streptomyces sp. S055
MLHRHAKASCAAFAAVGALVLAGLPATAAADAAPPAPPTAAATAGVDNASPGLLRAMQRDLGVTEAQARARLANEAEAGAVAGRLRISLGGDFAGAWVHGPDSAKLSVATTDASDRAAIEAGGAHAVVVRHTLPRLDGALAKLDEAASAAPSTAAEVPVRYVDVTANRVTLQTVRPAAAKALVVAAGVDPALVRVEKSAERPRPLYDLRGGDAYYMNGSGRCSVGFPVTKGTQQGFATAGHCGRVGTTTTGYNQVAQGSFQASTFPGRDMAWVATNSNWTATPYVKGNSGNVQVAGSTQAAVGASVCRSGSTTGWHCGTIQQHNTSVTYPEGTISGVTRTTVCAEPGDSGGSYISGSQAQGVTSGGSGNCRSGGTTYYQPINPLLQNYGLTLKTTSDDPGPGEPGEPGGTWAAGTVYAAGAQVTYGGATYRCLQGHQAQAGWEPPNVPALWQRA

SEQ ID NO: 13 - DNA, 1362 bp, artificial sequence
  synthesized gene
gtgagaagcaaaaaattgtggatcagcttgttgtttgcgttaacgttaatctttacgatggcgttcagcaacatgagcgcgcaggctgctggaaaagcaccggatggcaaaccggcagcaaaaacagcagcaagaacactggcagcaacagcagcgcaaccggaactgctgtcagcaatgcaaagagatctgggcctgacaagaccgcaagcacttacaagactggcaaatgaagcagaagcaggcgcaacagcggcaagactgagacaaggcctgggaggcgcatttgcaggcgcatgggttgatggaccggaatcaggcacactgacagttgcaacaacaagagcagcagatgcagcagcaattagagcaacaggcgcagatgcaagactggtttcacattcactgacagcactggaaagagcaaaaagaacacttgatggcgcagcgacagcggaagcaccggttagatatgttgatgttagagcaaatgttctggtcgttgaagaaacaagagctggcgcaggcgctagactggtcagagcgacaggcgttccgagagatcttgttagagttgttagaacaacatcagcaccgcgtccgctgtatgatattagaggcggagatgcgtattatatgggaggcggaggcagatgctcagttggctttgcagttacaagaggcacaacacaaggctttgcgacagcaggccattgcggcagagctggcacaacaacatctggctttaatcaagttgcacaaggctcatttcaaggctcaatttttccgggaaatgatatggcatgggtcgcagcaaatacaaattggacatcaacaccgtatgttaaaggctcaggcggagcgaatgttcaagttacaggctcagttctgcagccggttggcgcatcagtttgcagatcaggctcaacaacaggctggcattgcggaacaattcaacaacataatacaagcgtcacatatccggaaggcacaatttcaggcgttacaagaacaacagtttgcgcagaacctggcgattcaggcggatcatatatttcaggcagccaagcacaaggcgtgacatcaggcggaagcggcaattgctcatctggcggaacaacattttttcaaccgctgaatccgctgctgcaaaattatggcctgacactgaaaacaacaggatcagatccgggaccgggacctggcgaaccgcaaccgggtggaacatgggcagcaggcaaagtttatgcagcaggcgatacagttacatatggcggagctacatatagatgcctgcaaggccatcaagcacaaacaggatgggaaccgcctaatgttccggcactgtggcaaagacaataa

SEQ ID NO: 14 - DNA, 1404 bp, artificial sequence
  synthesized gene
gtgagaagcaaaaaattgtggatcagcttgttgtttgcgttaacgttaatctttacgatggcgttcagcaacatgagcgcgcaggctgctggaaaagcaccgtcagatggccatggaccggttgcagcaccgcctccggcagcaagatcagcagcagatgcactggcagtttcagcagttcaaccggatgttctgagagcaatgcaaagagatctgggcctgacaccggcagaagcaagacaaagactggcaaatgaagcagaagcaggcgcaacagcagcaagactgagacagagacttgcaggctcatatgcaggcgcatgggttgaaggccatgcatcatcagttctgacagttgcaacaacaagagcagatgatgcagcagcaattagagcatcaggcgctgaagcagatgttgttgcgcattcactggcagcactggatagaacaaaagcagcgcttgatagagcagcgagaacagcggctgatgttggcgttccggtttggtatgttgatgttagaacaaatagcgttgttgtccaagcagttgatcctggcgcagcagcatcactggttggcagagtttcagaagcagatagatcaagaattagagttgtcccgacaagagaaagaccgcgtccgctgtatgatattagaggcggagatgcgtattatatgggaggctcaggcagatgctcagttggcttttcagttacaagaggcacacaagcaggctttgcgacagcaggccattgcggcagagctggcacaacaacaacaggctataatagagttgcacaaggctcatttcaagcgtcaacatttccgggaagagatacagcatgggtcgcaacaaatacaaattggacagcaacaccgtatgttaaaggcgcaggcggagcaaatgttagagtcgcaggctcagttcaacagccggttggcgcatcagtttgcagatcaggcagcacaacaggatggcattgcggaacaattcaacaacataatacaagcgtgacatatccggaaggcacaattacaggcgttacaagaacaagcgtttgcgcagaacctggcgattcaggcggatcatatatttcaggctcacaagcacaaggcgtcacatctggcggatcaggcgattgcagaagcggaggcacaacatatcatcaaccgattaatccgctgctgcaagcatatggccttacactgacaacgacgacaggccctggcgatccgggaccgggtgatccggatgaaccgggtggcacatgggcagcaggcacagtttatagagcaggcgatcaagttacatatggcggagcgacatatagatgccttcaaggccatcaagcacaggcaggctgggaaccgcctaatgttccggcactgtggcaaagaggctaa

SEQ ID NO: 15 - DNA, 1377 bp, artificial sequence
  synthesized gene
gtgagaagcaaaaaattgtggatcagcttgttgtttgcgttaacgttaatctttacgatggcgttcagcaacatgagcgcgcaggctgctggaaaagcacaaccgtcacctggcgcagcgcctggcgcacaatcagcagcaagagcacttggcgcagcagaagcacaaccggaactgctggcagcaatgggcagagatctgggcctgacaagagcacaagcagaaagacgcctggttaatgaagcggaagcaggcgcagcggcagcaagactgagagatagaattggcggatcatttgcaggcgcatgggttgaaggcgcagaatcaggctcactgacagttgcaacaacaagagcagcagatctggatgcaattagagcggcaggcgcaacagcgagactggttagacatgatctgtcatcactggaaagagcaaaagcggcacttgatagagcagctggcgcagatgcaccggttagatatgttgatgttagagcaaatcaactggtcgtcgaagaagttagagcaggcgctggcgcaagactggcagcagcaacaggcgttccgagagaacttgttagagttgaaagatcaacagaagcaccgcgtccgctgtatgatctgagaggcggagatgcgtattatatgggaggcggaggcagatgctcagttggctttccggttacaagaggcacaacacaaggctttgcaacagcaggccattgcggcagagcaggccaaacaacatcaggctataatcaagttgcacaaggctcatttcaaggctcagtttttccgggatcagatatggcatgggtcgcagcaaattcatcatggacagcaacaccgtatgttaaaggcgcaggcggagcgaatgttcaagttacaggcagcgttctgcagccggttggcgcaagcgtttgcagatcaggatcaacaacaggctggcattgcggaacaattcaacaacataatacaagcgtcacatatccggaaggcacaatttcaggcgttacaagaacaacagtttgcgcagaacctggcgatagcggaggctcatatatttcaggcagccaagcacaaggcgtgacatcaggcggatcaggcaattgctcatctggcggaacaacatattttcaaccgctgaatccgattctgtcagcatatggcctgacactgaaaacaacgggcacagatccgggaccgggtccgggacctggcgaacctgaaccgggtggcacatggaaagcaggcacagtttatgctgcaggcgctacagttacatatggcggaagcacatatagatgcctgcaaggccatcaagcacaaacaggatgggaaccgcctaatgttccggcactgtggcaaagagtttaa

SEQ ID NO: 16 - DNA, 1374 bp, artificial sequence
  synthesized gene
gtgagaagcaaaaaattgtggatcagcttgttgtttgcgttaacgttaatctttacgatggcgttcagcaacatgagcgcgcaggctgctggaaaagcagcagatgcagcacctccggcacctcctacggcagcagcaacggcaggcgttgataatgcatcaccgggacttttaagagcaatgcaaagagatcttggagttacagaagcacaagcacgcgcacgccttgcaaacgaagcagaagcaggcgcagttgcaggacgccttagaattagcctcgggggcgattttgcaggcgcatgggttcatggccctgatagcgcaaaactgagcgttgcaacgacagatgcatcagatcgcgcagcaatcgaagctggcggcgcacatgcagttgttgttcgccatacactgcctagacttgatggcgcactggcaaaacttgatgaagcagcatctgcagcaccgtctacggcagcagaagttccggttcgctatgttgatgttacggcaaatagagttacacttcaaacggttcgccctgcagcagcaaaagcactggttgttgcagcaggcgttgatcctgcattagttcgcgttgaaaaatctgcagaaagacctagaccgctgtatgacttgcgaggcggagatgcatattatatgaatggctctggccgctgctcagttggctttcctgttacgaaaggaacacaacaaggatttgcaacggcaggccattgtggtcgtgttggaacgacgacgacaggctataatcaagttgcacaaggctcatttcaagcatctacatttccgggccgcgatatggcatgggttgcaacaaattctaattggacggcaacaccttatgttaaaggaaatagcggaaatgttcaagttgcaggctctacacaagcagcagttggcgcatctgtttgccgctctggatctacgacgggctggcattgcggaacaatccaacaacataatacgtcagttacgtatcctgaaggaacgatttctggagttacacgcacgacggtttgcgcagaaccgggagatagcggaggatcatatattagcggatctcaagcacaaggcgttacgagcggaggatctggaaattgccgctctggcggaacgacatattatcaacctattaatcctcttcttcaaaattatggactgacacttaaaacgacgtcagatgatccgggaccgggcgaaccgggagaaccgggcggaacatgggcagcaggaacagtttatgcagcaggcgcacaagttacgtatggaggcgcaacgtatcgctgcttacaaggccatcaagcacaagcaggctgggaacctccgaatgttccggcactgtggcaacgcgca

SEQ ID NO: 17 - protein, 29 aa, Bacillus subtilis
MRSKKLWISLLFALTLIFTMAFSNMSAQA

SEQ ID NO: 18 - protein, 191 aa, Streptomyces sp. C009
YDIRGGDAYYMGGSGRCSVGFSVTRGTQAGFATAGHCGRAGTTTTGYNRVAQGSFQASTFPGRDTAWVATNTNWTATPYVKGAGGANVRVAGSVQQPVGASVCRSGSTTGWHCGTIQQHNTSVTYPEGTITGVTRTSVCAEPGDSGGSYISGSQAQGVTSGGSGDCRSGGTTYHQPINPLLQAYGLTLTTT

SEQ ID NO: 19 - protein, 191 aa, Streptomyces sp. C001
YDLRGGDAYYMGGGGRCSVGFPVTRGTTQGFATAGHCGRAGQTTSGYNQVAQGSFQGSVFPGSDMAWVAANSSWTATPYVKGAGGANVQVTGSVLQPVGASVCRSGSTTGWHCGTIQQHNTSVTYPEGTISGVTRTTVCAEPGDSGGSYISGSQAQGVTSGGSGNCSSGGTTYFQPLNPILSAYGLTLKTT

SEQ ID NO: 20 - protein, 191 aa, Streptomyces sp. C003
YDIRGGDAYYMGGGGRCSVGFAVTRGTTQGFATAGHCGRAGTTTSGFNQVAQGSFQGSIFPGNDMAWVAANTNWTSTPYVKGSGGANVQVTGSVLQPVGASVCRSGSTTGWHCGTIQQHNTSVTYPEGTISGVTRTTVCAEPGDSGGSYISGSQAQGVTSGGSGNCSSGGTTFFQPLNPLLQNYGLTLKTT

SEQ ID NO: 21 - protein, 190 aa, Streptomyces sp. S055
YDLRGGDAYYMNGSGRCSVGFPVTKGTQQGFATAGHCGRVGTTTTGYNQVAQGSFQASTFPGRDMAWVATNSNWTATPYVKGNSGNVQVAGSTQAAVGASVCRSGSTTGWHCGTIQQHNTSVTYPEGTISGVTRTTVCAEPGDSGGSYISGSQAQGVTSGGSGNCRSGGTTYYQPINPLLQNYGLTLKTT

SEQ ID NO: 22 - protein, 435 aa, Streptomyces sp. C009
APSDGHGPVAAPPPAARSAADALAVSAVQPDVLRAMQRDLGLTPAEARQRLANEAEAGATAARLRQRLAGSYAGAWVEGHASSVLTVATTRADDAAAIRASGAEADVVAHSLAALDRTKAALDRAARTAADVGVPVWYVDVRTNSVVVQAVDPGAAASLVGRVSEADRSRIRVVPTRERPRPLYDIRGGDAYYMGGSGRCSVGFSVTRGTQAGFATAGHCGRAGTTTTGYNRVAQGSFQASTFPGRDTAWVATNTNWTATPYVKGAGGANVRVAGSVQQPVGASVCRSGSTTGWHCGTIQQHNTSVTYPEGTITGVTRTSVCAEPGDSGGSYISGSQAQGVTSGGSGDCRSGGTTYHQPINPLLQAYGLTLTTTTGPGDPGPGDPDEPGGTWAAGTVYRAGDQVTYGGATYRCLQGHQAQAGWEPPNVPALWQRG

SEQ ID NO: 23 - protein, 426 aa, Streptomyces sp. C001
AQPSPGAAPGAQSAARALGAAEAQPELLAAMGRDLGLTRAQAERRLVNEAEAGAAAARLRDRIGGSFAGAWVEGAESGSLTVATTRAADLDAIRAAGATARLVRHDLSSLERAKAALDRAAGADAPVRYVDVRANQLVVEEVRAGAGARLAAATGVPRELVRVERSTEAPRPLYDLRGGDAYYMGGGGRCSVGFPVTRGTTQGFATAGHCGRAGQTTSGYNQVAQGSFQGSVFPGSDMAWVAANSSWTATPYVKGAGGANVQVTGSVLQPVGASVCRSGSTTGWHCGTIQQHNTSVTYPEGTISGVTRTTVCAEPGDSGGSYISGSQAQGVTSGGSGNCSSGGTTYFQPLNPILSAYGLTLKTTGTDPGPGPGPGEPEPGGTWKAGTVYAAGATVTYGGSTYRCLQGHQAQTGWEPPNVPALWQRV

SEQ ID NO: 24 - protein, 421 aa, Streptomyces sp. C003
APDGKPAAKTAARTLAATAAQPELLSAMQRDLGLTRPQALTRLANEAEAGATAARLRQGLGGAFAGAWVDGPESGTLTVATTRAADAAAIRATGADARLVSHSLTALERAKRTLDGAATAEAPVRYVDVRANVLVVEETRAGAGARLVRATGVPRDLVRVVRTTSAPRPLYDIRGGDAYYMGGGGRCSVGFAVTRGTTQGFATAGHCGRAGTTTSGFNQVAQGSFQGSIFPGNDMAWVAANTNWTSTPYVKGSGGANVQVTGSVLQPVGASVCRSGSTTGWHCGTIQQHNTSVTYPEGTISGVTRTTVCAEPGDSGGSYISGSQAQGVTSGGSGNCSSGGTTFFQPLNPLLQNYGLTLKTTGSDPGPGPGEPQPGGTWAAGKVYAAGDTVTYGGATYRCLQGHQAQTGWEPPNVPALWQRQ

SEQ ID NO: 25 - protein, 426 aa, Streptomyces sp. S055
AADAAPPAPPTAAATAGVDNASPGLLRAMQRDLGVTEAQARARLANEAEAGAVAGRLRISLGGDFAGAWVHGPDSAKLSVATTDASDRAAIEAGGAHAVVVRHTLPRLDGALAKLDEAASAAPSTAAEVPVRYVDVTANRVTLQTVRPAAAKALVVAAGVDPALVRVEKSAERPRPLYDLRGGDAYYMNGSGRCSVGFPVTKGTQQGFATAGHCGRVGTTTTGYNQVAQGSFQASTFPGRDMAWVATNSNWTATPYVKGNSGNVQVAGSTQAAVGASVCRSGSTTGWHCGTIQQHNTSVTYPEGTISGVTRTTVCAEPGDSGGSYISGSQAQGVTSGGSGNCRSGGTTYYQPINPLLQNYGLTLKTTSDDPGPGEPGEPGGTWAAGTVYAAGAQVTYGGATYRCLQGHQAQAGWEPPNVPALWQRA

SEQ ID NO: 26 - protein, 453 aa, Streptomyces albulus
MFHRHARAACVGAVTVGALVLTALQGASAATPAPAARSAATTLAVSAVQPDVLRAMQRDLGLTGAEARVRLANEAEAGATAALLRQRLGGSFAGAWVEGDVSSVLTVATTRAADAAAIRASGAEADVVTHGLAQLDRTKAALDRAARTTATTGVPVWYVDVRTNSVVVGAADRAAAAALVARVGEADRSRIRVVPTRERPRPLYDIRGGDAYYMGGSGRCSVGFAITRGTQAGFATAGHCGRAGTTTTGYNQVAQGSFQASTFPGRDTAWVATSTNWTATPYVKGAGGANVQVTGSVQQPVGASVCRSGSTTGWHCGTIQQHNTSVTYPEGTISGVTRTSVCAEPGDSGGSYISGTQAQGVTSGGSGDCRSGGTTYHQPINPLLQAYGLTLRTTTDPDDPGDPGEPGGTWAAGTVYRAGDQVTYGGATYRCLQGHQAQAGWEPPNVPALWQRG

SEQ ID NO: 27 - protein, 456 aa, Streptomyces sp. NRRL F-5193
MLRRHARAACTALAAAGMVLAGLQAGSATAAQPSPGTGPGALSAARALGAAEARPELLAAMGRDLGLTRAQAERRLVNEAEAGAAAARLRDRLGGSFAGAWVAGAESGSLTVATTRAADLAAIRAAGAEAALVRHGLPALERAKAALDRAATADAPVRYVDIRANALVVEETRAGAGKRLVAATGVPAELVRVVRSAEAPRPLYDLRGGDAYYMGGGGRCSVGFPVTRGTTQGFATAGHCGRAGQTTSGYNQVAQGSFQASIFPGNDMAWVAANSNWTATPYVKGAGGANVQVTGSVLQPVGASVCRSGSTTGWHCGTIQQHNTSVTYPEGTISGVTRTTVCAEPGDSGGSYISGSQAQGVTSGGSGNCSSGGTTYFQPLNPILSNYGLTLKTTGTDPGPGPGPGEPEPGGTWKAGTVYAAGATVTYGGSTYRCLQGHQAQAGWEPPNVPALWQRV

SEQ ID NO: 28 - protein, 451 aa, Streptomyces exfoliatus
MLRRHTRAACTALVAAGMALAGLQAGTATAAPDGNPAAKTAAQTLAAGAAQPELLSAMQRDLGLTRPQALTRLANEAEAGATAARLRQGLGGAFAGAWVDGPESGTLTVATTRAADAAVIRATGANARLVSHTLTALERAKRALDGAATAEAPVRYVDVRANVLVVEETRPGAGARLVRATGVPRDLVRVVRTASAPRPLYDIRGGDAYYMGGGGRCSVGFAVTRGTTQGFATAGHCGRAGTTTSGFNQAAQGSFQGSIFPGNDMAWVAANTNWTSTPYVKGSGGANVQVTGSVLQPVGASVCRSGSTTGWHCGTIQQHNTSVTYPEGIISGVTRTTVCAEPGDSGGSYISGSQAQGVTSGGSGNCSSGGTTFFQPLNPLLQNYGLTLKTTGSDPGPGPGEPQPGGTWAAGKVYAAGDTVTYGGGTYRCLQGHQAQTGWEPPNVPALWQRQ

SEQ ID NO: 29 - protein, 459 aa, Streptomyces albus
MLHRHAKAAGVGAAAAALVLTTLQGAASAQQDGTPPGAVAHTAADTLAVSAAQPELLRAMQRDLGLTRAQAERRLVNEAEAGATAAVLRQRLGGSFAGAWVEGADSGTLTVATTRAADAAAIRAAGAESRTVTHNLAELDRTKAALDRAAERNSSTDVPVWYVDVRANAVVVRAVEKAAAQTLIEATAADRDRIRVVPTGEQPRPLYDIRGGDAYYMGGGGRCSVGFAVTRGTTQGFATAGHCGRAGTATSGYNQVAQGTFQASTFPGRDTAWVATNGNWTSTPYVKGQGGQNIQVTGSVQQPVGASICRSGSTTGWHCGTISQHNTSVTYPEGTISGVTRTTVCAEPGDSGGSYISGSQAQGVTSGGSGDCRSGGTTYHQPVNPLLQGYGLTLKTTVDPGDPGDPGDPGEPGGTWAAGTVYQAGAQVTYGGATYRCLQGHQAQAGWEPPNAPALWQRL

SEQ ID NO: 30 - protein, 453 aa, Streptomyces pristinaespiralis
MLHRHAKAACVGAAAAAALVLTSLQGAASAQPAGGAAPSAANTLAVSAAQPELLRAMQRDLGLTRAEAERRLVNEAEAGATAAVLRQRLGDSFAGAWVEGADSGTLTVATTRAADAAAIRAGGAEARTVTHTLAELDRAKAALDRAAARDSSTDVPVWYVDVRANAVVVRAVERSAAQTLIGASGAERDLIRVVPTEEQPRPLYDIRGGDAYYMGGGGRCSVGFAVTRGTTQGFATAGHCGRAGTSTSGYNQVAQGTFQASTFPGRDTAWVAANSNWTSTPYVKGQSGQNIQVTGSVQQPVGASICRSGSTTGWHCGTISQHNTSVTYPEGTITGVTRTTVCAEPGDSGGSYISGSQAQGVTSGGSGDCRSGGTTFHQPINPLLQGYGLTLKTTTDPGEPGEPGEPGGTWAAGTVYQAGAQVTYGGVTYRCLQGHQAQAGWEPPNVPALWQRL

SEQ ID NO: 31 - protein, 451 aa, Streptomyces venezuelae
MLRRHARAACTALVAAGMALAGLQAGTATAAPDGKPAAKTAARTLATAAAQPELLSAMQRDLGLTRPQALTRLANEAEAGATAAGLRQSLAGAFAGAWVDGAESGTLTVATTRSADTAAIRATGANARLVPHSLTALERAKRALDRGATAEAPVRYVDVRANVLVVEETRAGAGARLVEAAGVPRDLVRVVRTDRAPRPLYDIRGGDAYYMGGGGRCSVGFAVTRGATQGFATAGHCGRAGTTTSGFNQVAQGSFQASTFPGNDMAWVAANTNWTSTPYVKGSGGANVQVTGSVLQPVGASVCRSGSTTGWHCGTIQQHNTSVTYPEGTISGVTRTTVCAEPGDSGGSYISGSQAQGVTSGGSGNCSSGGTTFFQPLNPLLQNYGLTLKTTGTDPGPGPGEPQPGGTWAAGKVYAAGATVTYGGATYRCLQGHQAQTGWEPPNVPALWQRQ

SEQ ID NO: 32 - protein, 456 aa, Streptomyces venezuelae
MLRRHARAACTALVAAGLALAGLQAGTAAAVPDAGEPHPAPTVRTAAQTLGADAAAPEVLDAMRRDLGLSHAQALTRLANEAEAGATAARLRQGLGGAFAGAWVDGAESGTLTVATTRSSDAAAIRATGARARLVTHPLTILERAKERLDRAATADAPVRYVDVRANVLVVEETKAGAGARLLAATGVPRELVRVVRSGQAPRPLYDLRGGDAYYMGGGGRCSVGFPVTRGTTQGFATAGHCGRAGTTTSGFNQVAQGSFQGSIFPGNDMAWVAANGNWTATPYVKGSGGANVQVTGSVLQPVGSSVCRSGSTTGWHCGTVQQHNTSVTYPEGTISGVTRTTVCAEPGDSGGSYISGSQAQGVTSGGSGNCSSGGTTFFQPLNPLLQNYGLTLKTTGGDPGPGPGEPEPGGSWAAGTVYKAGDVVTYGGASYRCLQGHQAQAGWQPPNVPALWQRL

SEQ ID NO: 33 - protein, 455 aa, unknown
  Streptomyces sp.
MLKRHARAACTALAAAGLLLAGTTAAGAATPAADPAAPQKSATATLRPGDAPAELLTALQRDLGLTPTQAKDRLAHEAEAGATAARLRARLGAAFAGAWVDGADSATLTVATTRAADAAAIRAAGAEAKLVSRSLADLDAVRAGLDRAATAETPVRYVDPRTNTLVVEETRPGAAAGLLAATGTDPALATVVRTAAEQAPRPLYDLRGGDAYYMNGQGRCSVGFPVTRGTTQGFATAGHCGRAGTTTSGFNQVAQGSFQASVFPGNDMAWVAANTSWTATPYVKGSGGANVQVTGSVLQPVGSSVCRSGSTTGWHCGTVQQHNTSVTYPEGTISGVTRTTVCAEPGDSGGSYISGSQAQGVTSGGSGDCTSGGTTFFQPLNPILSAYGLTLKVTGSDPGPGPGPGPQPGGTWKAGTVYAAGDTVTYGGAAYRCLQGHQAQTGWEPSVVPALWQKL

SEQ ID NO: 34 - protein, 456 aa, Streptomyces flavochromogenes
MLRRHARAACTSLVAAGMALAGLQAGSAAAAPDGRDPHRAPTVRTAAQALGADSARPELLDAMRRDLGLTHSQARTRLANEAEAGATAARLRQGLGGAFAGAWVDGAEAGTLTVATTRAADTPAIRATGARARLVTHSLTALERAKQRLDRTAGTDAPVRYVDVRANVLVVEETRAGAGARLVRATGVPRELVRVVRTGQAPRPLYDIRGGDAYYMGGGGRCSVGFAVTRGTTQGFATAGHCGRAGTTTSGFNQVAQGSFQGSVFPGNDMAWVAANTNWTSTPYVKGSGGANVQVTGSVLQPVGSSVCRSGSTTGWHCGTVQQHNTSVTYPEGTISGVTRTTVCAEPGDSGGSYISGSQAQGVTSGGSGNCSSGGTTFFQPLNPLLQNYGLTLKTTGTDPGPGPGEPEPGGTWAAGKVYAAGDVVTYGGASYRCLQGHQAQAGWQPPNVPALWQRL

SEQ ID NO: 35 - protein, 454 aa, Streptomyces bikiniensis
MLRRHARTACTVLAAAGLALAGLQAGSATAAPHDKDRAPTVRTAADALGAASAQPELLRAMQRDLGLTRAQAENRLANEAEAGARAAGLRLDLGGAFAGAWVDGAESGTLTVATTRAADAAAIRAAGAHAEVVTHGLSALERAKEALDRAATADVPVRYVDVRANVLVVEEARAGAGARLVAATGVPRDLVKVVRTAEAPRPLYDLRGGDAYYMNGSGRCSVGFPITKGTTQGFATAGHCGRAGTSTSGYNQVAQGSFQASVFPGSDMAWVATNSNWTATPYVKGAGGANVQVTGSVLQPVGSSVCRSGSTTGWHCGTVQQHNTSVTYPEGTISGVTRTTVCAEPGDSGGSYISGSQAQGVTSGGSGNCSSGGTTFFQPLNPILQNYGLTLKTTTDPGPGPGPEPEPGGTWAAGKVYKAGDTVTYGGSTYRCLQGHQAQTGWEPSNVPALWQRV

SEQ ID NO: 36 - protein, 456 aa, Streptomyces griseus
MLRRHARAACTALAAAGMALAGLQAGSATAAQPSPGSAPSVRSAAHALGADEARPELLAAMGRDLGLTRTQAEHRLVNEAEAGATAARLRQHLGGSFAGAWVAGAESGSLTVATTRAADLAAIRAAGAEAALVRHGLTALERARTALDRAATADAPVRYVDVRANVLVVEETRAGAGDRLVAATGVPRELVRVVRSAGAPRPLYDIRGGDAYYMGGGGRCSVGFPVTRGTTQGFATAGHCGRAGQSTSGFNQVAQGTFQASVFPGNDMAWVAAHTNWTSTPYVKGAGGANVQVTGSVLQPVGSSVCRSGSTTGWHCGTIQQHNTSVTYQEGTVSGVTRTTVCAEPGDSGGSYISGSQAQGVTSGGTGNCSSGGTTYFQPLNPILSGYGLTLKTTGSDPGPGPGPGEPQPGGTWTAGKVYAAGDTVTYGGSTYRCLQAHQAQAGWEPSNVPALWQRI

SEQ ID NO: 37 - protein, 454 aa, Streptomyces sp. NRRL_F-5727
MLRRHARAACTVLAAAGMVLAGLQAGSATAAPPSPGTAPAVQSAARALGAGEARPELLAAMGRDLGLTRAQAERRLVNEAEAGATAARLRDRLGGAFAGAWVEGAESGSLTVATTRAADLAAIRAAGATARLVRHDLAALERAKESLDRAAGADAPVRYVDVRANVLVVEETRAGAGARLAAETGVARELVRVVRSAEAPRPLYDLRGGDAYYMGGGGRCSVGFPVTRGTTQGFATAGHCGRAGQSTSGYNQVAQGTFQASVFPGNDMAWVAANSSWTATPYVKGAGGANVQVTGSVLQPVGASVCRSGSTTGWHCGTIQQHNTSVTYPEGTISGVTRTTVCAEPGDSGGSYISGSQAQGVTSGGSGNCSSGGTTFFQPLNPILSNYGLTLRTAGTDPGPGPGEPEPGGTWKAGTVYAAGATVTYGGSTYRCLQGHQAQAGWEPPNVPALWQRV

SEQ ID NO: 38 - protein, 458 aa, Streptomyces sp. CNT372
MFHRPVRAACAAAVAAGALVLASLQGYAAAQPSAASPGGGTSAAGTLAVSAAQPGLKRAMQRDLGLTAAQAERRLVNEAEAGAAAARLQQRLGASFAGAWVSGAESGTLTVATTRAADHAAVRAEGAKPVAAGASLAELDRAKAALDRAAARAGSPAVPVWYVDVRENTVVVGARNTAAAEALIAASGADRDRIRVAATGESPRPLYDIRGGDAYYMGGGGRCSVGFAVTRGTQHGFATAGHCGRAGTATSGFNQVAQGTFQASTFPGRDTAWVLTNSQWTATPYVKGAGGQNVQVAGSVQQPVGASVCRSGSTTGWHCGTIQQHNTSVTYPEGTITGVTRTSVCAEPGDSGGSYISGSQAQGVTSGGSGNCRTGGTTYHQPINPLLQAYGLTLRTTTDPGPGPGEPPGEPGGTWAAGTVYATGAQVTYGGATYRCLQGHQAQTGWEPPNVPALWQRL

SEQ ID NO: 39 - protein, 191 aa, Streptomyces griseus
ADIRGGDAYYMNGSGRCSVGFSVTRGTQNGFATAGHCGRVGTTTNGVNQQAQGTFQGSTFPGRDIAWVATNANWTPRPLVNGYGRGDVTVAGSTASVVGASVCRSGSTTGWHCGTIQQLNTSVTYPEGTISGVTRTSVCAEPGDSGGSYISGSQAQGVTSGGSGNCSSGGTTYFQPINPLLQAYGLTLVTS

SEQ ID NO: 40 - protein, 275 aa, Bacillus amyloliquefaciens
AQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGALNGTSMASPHVAGAAALILSKHPNWTNTQVRSSLENTTTKLGDSFYYGKGLINVQAAAQ

SEQ ID NO: 41 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 42 - protein, 191 aa, Streptomyces albulus
YDIRGGDAYYMGGSGRCSVGFAITRGTQAGFATAGHCGRAGTTTTGYNQVAQGSFQASTFPGRDTAWVATSTNWTATPYVKGAGGANVQVTGSVQQPVGASVCRSGSTTGWHCGTIQQHNTSVTYPEGTISGVTRTSVCAEPGDSGGSYISGTQAQGVTSGGSGDCRSGGTTYHQPINPLLQAYGLTLRTT

SEQ ID NO: 43 - protein, 191 aa, Streptomyces sp. NRRL F-5193
YDLRGGDAYYMGGGGRCSVGFPVTRGTTQGFATAGHCGRAGQTTSGYNQVAQGSFQASIFPGNDMAWVAANSNWTATPYVKGAGGANVQVTGSVLQPVGASVCRSGSTTGWHCGTIQQHNTSVTYPEGTISGVTRTTVCAEPGDSGGSYISGSQAQGVTSGGSGNCSSGGTTYFQPLNPILSNYGLTLKTT

SEQ ID NO: 44 - protein, 191 aa, Streptomyces exfoliatus
YDIRGGDAYYMGGGGRCSVGFAVTRGTTQGFATAGHCGRAGTTTSGFNQAAQGSFQGSIFPGNDMAWVAANTNWTSTPYVKGSGGANVQVTGSVLQPVGASVCRSGSTTGWHCGTIQQHNTSVTYPEGIISGVTRTTVCAEPGDSGGSYISGSQAQGVTSGGSGNCSSGGTTFFQPLNPLLQNYGLTLKTT

SEQ ID NO: 45 - protein, 191 aa, Streptomyces albus
YDIRGGDAYYMGGGGRCSVGFAVTRGTTQGFATAGHCGRAGTATSGYNQVAQGTFQASTFPGRDTAWVATNGNWTSTPYVKGQGGQNIQVTGSVQQPVGASICRSGSTTGWHCGTISQHNTSVTYPEGTISGVTRTTVCAEPGDSGGSYISGSQAQGVTSGGSGDCRSGGTTYHQPVNPLLQGYGLTLKTT

SEQ ID NO: 46 - protein, 191 aa, Streptomyces pristinaespiralis
YDIRGGDAYYMGGGGRCSVGFAVTRGTTQGFATAGHCGRAGTSTSGYNQVAQGTFQASTFPGRDTAWVAANSNWTSTPYVKGQSGQNIQVTGSVQQPVGASICRSGSTTGWHCGTISQHNTSVTYPEGTITGVTRTTVCAEPGDSGGSYISGSQAQGVTSGGSGDCRSGGTTFHQPINPLLQGYGLTLKTT

SEQ ID NO: 47 - protein, 191 aa, Streptomyces leeuwenhoekii
YDLRGGDAYYMNGSGRCSVGFPVTRGTQQGFATAGHCGRAGTSTTGYNQAAQGTFQASTFPGRDMAWVATNSNWTATPYVKGAGGQNVQVTGSTQAPVGSSVCRSGSTTGWHCGTIQQLNTSVTYPEGTITGVTRTSVCAEPGDSGGSYISGSQAQGVTSGGSGNCRTGGTTYFQPLNPLLQSYGLTLKTN

SEQ ID NO: 48 - protein, 191 aa, Streptomyces sp. CNT372
YDIRGGDAYYMGGGGRCSVGFAVTRGTQHGFATAGHCGRAGTATSGFNQVAQGTFQASTFPGRDTAWVLTNSQWTATPYVKGAGGQNVQVAGSVQQPVGASVCRSGSTTGWHCGTIQQHNTSVTYPEGTITGVTRTSVCAEPGDSGGSYISGSQAQGVTSGGSGNCRTGGTTYHQPINPLLQAYGLTLRTT

SEQ ID NO: 49 - protein, 191 aa, Streptomyces cyaneogriseus
YDLRGGDAYYMNGSGRCSVGFPITRGTQQGFATAGHCGRAGTSTTGYNQAAQGTFQASTFPGRDMAWVATNSNWTATPYVKGAGGQNVQVAGSTQAPVGSSVCRSGSTTGWHCGTVQQLNTSVTYPEGTITGVTRTSVCAEPGDSGGSYISGSQAQGVTSGGSGDCRTGGTTYFQPINPLLQTYGLTLRTN

SEQ ID NO: 50 - protein, 191 aa, Streptomyces niveus
YDIRGGDAYYMNGSGRCSVGFAVTKGTQQGFATAGHCGRPGTTTSGYNQVAQGSFQASTFPGRDTAWVATNTNWTSTPYVKGQGAANVQVTGSTQSPVGASVCRSGSTTGWHCGVIQQHNTSVTYPEGTISGVTRTTVCAEPGDSGGSYISGSQAQGVTSGGSGNCSSGGTTFYQPVNPLLQAYALTLKTT

SEQ ID NO: 51 - protein, 191 aa, Streptomyces venezuelae
YDIRGGDAYYMGGGGRCSVGFAVTRGATQGFATAGHCGRAGTTTSGFNQVAQGSFQASTFPGNDMAWVAANTNWTSTPYVKGSGGANVQVTGSVLQPVGASVCRSGSTTGWHCGTIQQHNTSVTYPEGTISGVTRTTVCAEPGDSGGSYISGSQAQGVTSGGSGNCSSGGTTFFQPLNPLLQNYGLTLKTT

SEQ ID NO: 52 - protein, 190 aa, Streptomyces sp. NRRL F-5755
YDIRGGDAYYMGGGRCSVGFAITRGSQQGFATAGHCGRAGTSTTGYNQVAQGSFQASTFPGRDTAWVAANSNWTSTPYVKGQGGQNVRVAGSTQAAVGASICRSGSTTGWHCGTIQQHNTSVTYPQGTVSGVTRTSVCAEPGDSGGSFISGSQAQGVTSGGSGNCRSGGTTFYQPINPLLQGYGLTLKTG

SEQ ID NO: 53 - protein, 191 aa, Streptomyces fradiae
YDIRGGDAYHMGGGGRCSVGFAVTKGTQHGFATAGHCGRVGTSTSGYNQVAQGTFQGSTFPGRDMAWVAANTNWRSTPYVKGAGGQNVQVTGSTQAVVGASVCRSGSTTGWHCGTIQQHNTSVTYPEGTISGVTRTTVCAEPGDSGGSYISGSQAQGVTSGGSGDCRTGGTTYHQPLNPLLQAYALTLTTT

SEQ ID NO: 54 - protein, 191 aa, artificial sequence
  predicted catalytic domain consensus sequence
  MISC_FEATURE (3)..(3): X = I or L
  MISC_FEATURE (165)..(165): B is D or N
  MISC_FEATURE (177)..(177): X= I or L
YDXRGGDAYYMGGGGRCSVGFAVTRGTQQGFATAGHCGRAGTTTTGYNQVAQGSFQASTFPGRDTAWVATNTNWTATPYVKGAGGANVQVAGSVQQPVGASVCRSGSTTGWHCGTIQQHNTSVTYPEGTISGVTRTTVCAEPGDSGGSYISGSQAQGVTSGGSGBCRSGGTTYHQPXNPLLQAYGLTLTTT